(19) United States
(12) Patent Application Publication
(10) Pub. No.: US 2025/0041310 A1
(43) Pub. Date: Feb. 6, 2025

(54) Combination Therapy Comprising A2A/A2B and PD-1/PD-L1 Inhibitors

(71) Applicant: Incyte Corporation, Wilmington, DE (US)

(72) Inventors: Hui Wang, Hockessin, DE (US); Peter Niels Carlsen, Claymont, DE (US); Taisheng Huang, Wilmington, DE (US); Yong Li, Newark, DE (US); Luping Lin, Garnet Valley, PA (US); Chao Qi, Newark, DE (US); Pramod Unnikrishnan Thekkat, Chadds Ford, PA (US); Xiaozhao Wang, Moorestown, NJ (US); Liangxing Wu, Wilmington, DE (US); Wenqing Yao, Chadds Ford, PA (US); Wenyu Zhu, Media, PA (US)

(21) Appl. No.: 18/768,109

(22) Filed: Jul. 10, 2024

Related U.S. Application Data
(63) Continuation of application No. 17/138,485, filed on Dec. 30, 2020.
(60) Provisional application No. 62/956,960, filed on Jan. 3, 2020.

(51) Int. Cl. A61K 31/519; A61K 31/4985; A61K 31/501; A61K 31/506; A61K 39/00; A61K 39/395; A61P 35/00

(52) U.S. Cl. CPC A61K 31/519; A61K 31/4985; A61K 31/501; A61K 31/506; A61K 39/3955; A61P 35/00; A61K 2039/505; A61K 2039/545

(57) ABSTRACT

The present application provides methods of treating cancer using a combination of an inhibitor of A2A and/or A2B and an inhibitor of PD-1 and/or PD-L1.

### TECHNICAL FIELD

Disclosed herein are combination therapies comprising an inhibitor of A2A/A2B and an inhibitor of PD-1/PD-L1, and methods of using the same to treat disorders such as cancer.

### SEQUENCE LISTING

This application contains a Sequence Listing that has been submitted electronically as an XML file named “20443-0660002_SL_ST26.xml.” The XML file, created on Jun. 25, 2024, is 16,384 bytes in size. The material in the XML file is hereby incorporated by reference in its entirety.

### BACKGROUND

Some cancer patients have poor long-term prognosis and/or are resistant to one or more types of treatment commonly used in the art. Therefore, a need remains for effective therapies for cancer with increased efficacy and improved safety profiles in this difficult-to-treat patient population.

### SUMMARY

The present application provides, inter alia, a method of treating a cancer in a subject, comprising administering to the subject:
(i) an inhibitor of A2A/A2B; and
(ii) an inhibitor of PD-1/PD-L1.

Other features, objects, and advantages of the invention will be apparent from the description and from the claims.

### BRIEF DESCRIPTION OF THE DRAWINGS

FIGS. 1A-IC shows the synergistic effect of Compound 9 with (1A) pembrolizumab, (1B) Antibody X and (1C) Compound Y in CHO-PD-L1 co-cultured with primary T cells (See Example 1).

FIGS. 2A-2D shows the synergistic effect of Compound 9 or Compound 3A with atezolizumab in PBMC stimulated with CD3 antibody.

FIGS. 3A-3C shows the anti-tumor effect of Compound 9 and anti-PD1 (clone 29F.1A12 against murine PD-1) in preclinical CT26 and B16-F10 tumor models. (3A) Efficacy study of 10 mg/kg BID Compound 9 in CT26 syngeneic model as single agent and in combination with anti-PD1 antibody. (3B) Efficacy study of 10 mg/kg BID Compound 9 in CT-26 NSG xenograft model. (3C) Efficacy study of 10 mg/kg BID Compound 9 in B16 syngeneic model as single agent and in combination with anti-PD-L1 antibody.

### DETAILED DESCRIPTION

The present application provides a method of treating cancer in a subject, comprising administering to the subject:
(i) an inhibitor of A2A/A2B; and
(ii) an inhibitor of PD-1/PD-L1.

### A2A/A2B Inhibitors

Adenosine is an extracellular signaling molecule that can modulate immune responses through many immune cell types. Adenosine was first recognized as a physiologic regulator of coronary vascular tone by Drury and Szent-Györgyu (Sachdeva, S. and Gupta, M. Saudi Pharmaceutical Journal, 2013, 21, 245-253), however it was not until 1970 that Sattin and Rall showed that adenosine regulates cell function via occupancy of specific receptors on the cell surface (Sattin, A., and Rall, T. W., 1970. Mol. Pharmacol. 6, 13-23; Hasko', G., at al., 2007, Pharmacol. Ther. 113, 264-275).

Adenosine plays a vital role in various other physiological functions. It is involved in the synthesis of nucleic acids, when linked to three phosphate groups; it forms ATP, the integral component of the cellular energy system. Adenosine can be generated by the enzymatic breakdown of extracellular ATP, or can be also released from injured neurons and glial cells by passing the damaged plasma membrane (Tautenhahn, M. et al. Neuropharmacology, 2012, 62, 1756-1766). Adenosine produces various pharmacological effects, both in periphery and in the central nervous system, through an action on specific receptors localized on cell membranes (Matsumoto, T. et al. Pharmacol. Res., 2012, 65, 81-90). Alternative pathways for extracellular adenosine generation have been described. These pathways include the production of adenosine from nicotinamide dinucleotide (NAD) instead of ATP by the concerted action of CD38, CD203a and CD73. CD73-independent production of adenosine can also occur by other phosphates such as alkaline phosphatase or prostate-specific phosphatase.

There are four known subtypes of adenosine receptor in humans including A1, A2A (ADORA2A), A2B (ADORA2B), and A3 receptors. A1 and A2A are high affinity receptors, whereas A2B and A3 are low affinity receptors. Adenosine and its agonists can act via one or more of these receptors and can modulate the activity of adenylate cyclase, the enzyme responsible for increasing cyclic AMP (cAMP). The different receptors have differential stimulatory and inhibitory effects on this enzyme. Increased intracellular concentrations of cAMP can suppress the activity of immune and inflammatory cells (Livingston, M. et al., Inflamm. Res., 2004, 53, 171-178).

The A2A adenosine receptor can signal in the periphery and the CNS, with agonists explored as anti-inflammatory drugs and antagonists explored for neurodegenerative diseases (Carlsson, J. et al., J. Med. Chem., 2010, 53, 3748-3755). In most cell types the A2A subtype inhibits intracellular calcium levels whereas the A2B potentiates them. The A2A receptor generally appears to inhibit inflammatory response from immune cells (Borrmann, T. et al., J. Med. Chem., 2009, 52(13), 3994-4006).

A2B receptors are highly expressed in the gastrointestinal tract, bladder, lung and on mast cells (Antonioli, L. et al., Nature Reviews Cancer, 2013, 13, 842-857). The A2B receptor, although structurally closely related to the A2A receptor and able to activate adenylate cyclase, is functionally different. It has been postulated that this subtype may utilize signal transduction systems other than adenylate cyclase (Livingston, M. et al., Inflamm. Res., 2004, 53, 171-178). Among all the adenosine receptors, the A2B adenosine receptor is considered a low affinity receptor that is thought to remain silent under physiological conditions and to be activated as a consequence of increased extracellular adenosine levels (Ryzhov, S. et al. Neoplasia, 2008, 10, 987-995). Activation of A2B adenosine receptor can stimulate adenylate cyclase and phospholipase C through activation of Gs and Gq proteins, respectively. Coupling to mitogen activated protein kinases has also been described (Borrmann, T. et al., J. Med. Chem., 2009, 52(13), 3994-4006).

In the immune system, engagement of adenosine signaling can be a critical regulatory mechanism that protects tissues against excessive immune reactions. Adenosine can negatively modulate immune responses through many immune cell types, including T-cells, natural-killer cells, macrophages, dendritic cells, mast cells and myeloid-derived suppressor cells (Allard, B. et al. Current Opinion in Pharmacology, 2016, 29, 7-16).

In tumors, this pathway is hijacked by the tumor micro-environment and sabotages the antitumor capacity of the immune system, promoting cancer progression. In the tumor micro-environment, adenosine is mainly generated from extracellular ATP by two ectonucleotidases CD39 and CD73. Multiple cell types can generate adenosine by expressing CD39 and CD73. This is the case for tumor cells, T-effector cells, T-regulatory cells, tumor associated macrophages, myeloid derived suppressive cells (MDSCs), endothelial cells, cancer-associated fibroblast (CAFs) and mesenchymal stromal/stem cells (MSCs). Additionally, hypoxia and inflammation, conditions common to the tumor micro-environment, induces expression of CD39 and CD73, leading to increased adenosine production. As a result, the adenosine level in solid tumors is higher compared to normal physiological conditions.

A2A are mostly expressed on lymphoid-derived cells, including T-effector cells, T regulatory cells and natural killer (NK) cells. Blocking A2A receptor can prevent downstream immunosuppressive signals that temporarily inactivate T cells. A2B receptors are mainly expressed on monocyte-derived cells including dendritic cells, tumor-associated macrophages, myeloid derived suppressive cells (MDSCs), and mesenchymal stromal/stem cells (MSCs). Blocking A2B receptor in preclinical models can suppress tumor growth, block metastasis, and increase the presentation of tumor antigens.

In terms of safety profile of ADORA2A/ADORA2B (A2A/A2B) blockage, the A2A and A2B receptor knockout (KO) mice are all viable, showing no growth abnormalities and are fertile (Allard, B. et al. Current Opinion in Pharmacology, 2016, 29, 7-16). A2A KO mice displayed increased levels of pro-inflammatory cytokines only upon challenge with lipopolysaccharides (LPS) and no evidence of inflammation at baseline (Antonioli, L. et al., Nature Reviews Cancer, 2013, 13, 842-857). A2B KO mice exhibited normal platelet, red blood, and white blood cell counts but increased inflammation at baseline such as TNF-alpha andIL-6)(Antonioli, L. et al., Nature Reviews Cancer, 2013, 13, 842-857). A further increase in production of TNF-alpha and IL-6 was detected following LPS treatment. A2B KO mice also exhibited increased vascular adhesion molecules that mediate inflammation as well leukocyte adhesion/rolling; enhanced mast-cell activation; increased sensitivity to IgE-mediated anaphylaxis and increased vascular leakage and neutrophil influx under hypoxia (Antonioli, L. et al., Nature Reviews Cancer, 2013, 13, 842-857).

Adenosine pathway is a critical immune suppressive pathway that protects tissues against excessive immune reactions (Antonioli, L. et al. Nature Review Cancer. 2013, 13, 842-857; Inflamm. Res. 2004, 53: 171-178; Allard, et al. Current Opinion in Pharmacology 2016, 29:7). The immunosuppressive activity of adenosine is mediated through two G-protein coupled receptors (GPCRs) known as A2A and A2B; both receptors are found expressed on many immune cell types, including T-cells, natural-killer cells, macrophages, dendritic cells, mast cells and myeloid-derived suppressor cells (Saudi Pharmaceutical Journal. 2013, 21:245; Frontiers in Immunology. 2019, 10:925; J Clin Invest. 2017, 127(3):929; Neoplasia. 2008, 10: 987; Neoplasia. 2013, 15:1400). As a consequence of the high levels of adenosine production observed in the tumor microenvironment, it has been reported that the antitumor capacity of the immune system is suppressed resulting in cancer progression.

In some embodiments, the inhibitor of A2A/A2B is a compound selected from Table 1, or a pharmaceutically acceptable salt thereof.

TABLE 1

| Comp. | | |
|---|---|---|
| No. | Name | Structure |
| 1 | 3-(5-Amino-2-(pyridin-2-ylmethyl)-8- (pyrimidin-4-yl)-[1,2,4]triazolo[1,5- c]pyrimidin-7-yl)benzonitrile | 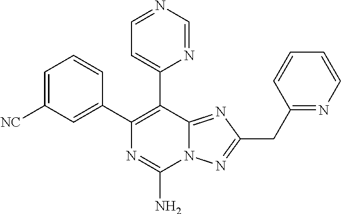 [SMILES: N#Cc1cccc(-c2nc(N)n3nc(Cc4ccccn4)nc3c2-c2ccncn2)c1] |
| 2 | 3-(5-Amino-2-((2,6- difluorophenyl)(hydroxy)methyl)-8- (pyrimidin-4-yl)-[1,2,4]triazolo[1,5- c]pyrimidin-7-yl)benzonitrile | 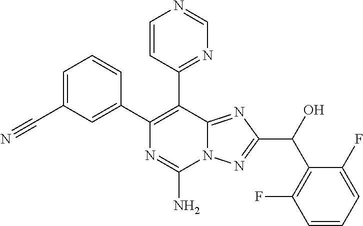 [SMILES: N#Cc1cccc(-c2nc(N)n3nc(C(O)c4c(F)cccc4F)nc3c2-c2ccncn2)c1] |
| 3A | 3-(5-amino-2-((5-(pyridin-2-yl)-2H- tetrazol-2-yl)methyl)-8-(pyrimidin-4-yl)- [1,2,4]triazolo[1,5-c]pyrimidin-7- yl)benzonitrile | 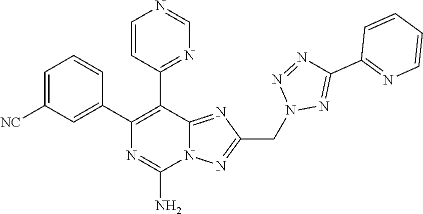 [SMILES: N#Cc1cccc(-c2nc(N)n3nc(Cn4nnc(-c5ccccn5)n4)nc3c2-c2ccncn2)c1] |
| 3B | 3-(5-Amino-2-((5-(pyridin-2-yl)-1H- tetrazol-1-yl)methyl)-8-(pyrimidin-4-yl)- [1,2,4]triazolo[1,5-c]pyrimidin-7- yl)benzonitrile | 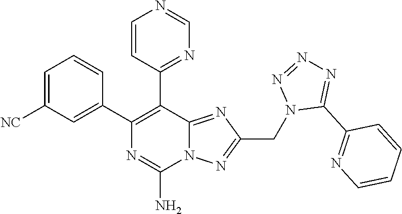 [SMILES: N#Cc1cccc(-c2nc(N)n3nc(Cn4nnnc4-c4ccccn4)nc3c2-c2ccncn2)c1] |
| 4 | 3-(5-Amino-2-((3-methylpyridin-2- yl)methoxy)-8-(pyrimidin-4-yl)- [1,2,4]triazolo[1,5-c]pyrimidin-7- yl)benzonitrile | 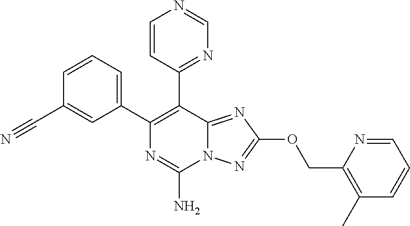 [SMILES: Cc1cccnc1COc1nc2c(-c3ccncn3)c(-c3cccc(C#N)c3)nc(N)n2n1] |
| 5 | 3-(5-Amino-2-(hydroxy(phenyl)methyl)- [1,2,4]triazolo[1,5-c]pyrimidin-7- yl)benzonitrile | 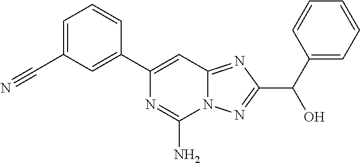 [SMILES: N#Cc1cccc(-c2cc3nc(C(O)c4ccccc4)nn3c(N)n2)c1] |
| 6 | 3-(5-Amino-2-((2,6- difluorophenyl)(hydroxy)methyl)- [1,2,4]triazolo[1,5-c]pyrimidin-7-yl)-2- fluorobenzonitrile | 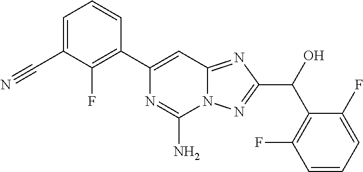 [SMILES: N#Cc1cccc(-c2cc3nc(C(O)c4c(F)cccc4F)nn3c(N)n2)c1F] |
| 7 | 5-Amino-7-(3-cyano-2-fluorophenyl)-2- ((2,6-difluorophenyl)(hydroxy)methyl)- [1,2,4]triazolo[1,5-c]pyrimidine-8- carbonitrile | 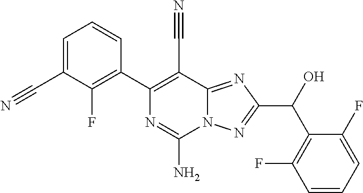 [SMILES: N#Cc1cccc(-c2nc(N)n3nc(C(O)c4c(F)cccc4F)nc3c2C#N)c1F] |
| 8 | 3-(5-Amino-2-((2-fluoro-6-(((1-methyl-2- oxopyrrolidin-3-yl)amino) methyl)phenyl)(hydroxy)methyl)- [1,2,4]triazolo[1,5-c]pyrimidin-7-yl)-2- fluorobenzonitrile | 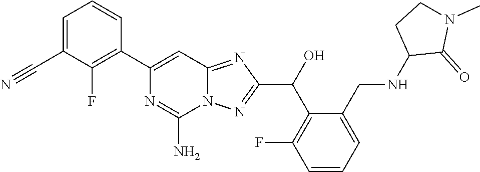 [SMILES: CN1CCC(NCc2cccc(F)c2C(O)c2nc3cc(-c4cccc(C#N)c4F)nc(N)n3n2)C1=O] |
| 9 | 3-(8-Amino-5-(1-methyl-6-oxo-1,6- dihydropyridazin-3-yl)-2-(pyridin-2- ylmethyl)-[1,2,4]triazolo[1,5-a]pyrazin-6- yl)benzonitrile | 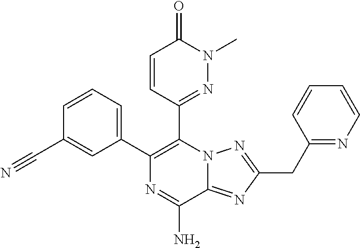 [SMILES: Cn1nc(-c2c(-c3cccc(C#N)c3)nc(N)c3nc(Cc4ccccn4)nn23)ccc1=O] |
| 10 | 3-(8-Amino-2-((2,6- difluorophenyl)(hydroxy)methyl)-5- (pyrimidin-4-yl)-[1,2,4]triazolo[1,5- a]pyrazin-6-yl)benzonitrile | 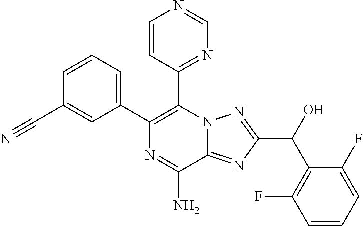 [SMILES: N#Cc1cccc(-c2nc(N)c3nc(C(O)c4c(F)cccc4F)nn3c2-c2ccncn2)c1] |
| 11 | 3-(8-amino-2-(amino(2,6- difluorophenyl)methyl)-5-(4- methyloxazol-5-yl)-[1,2,4]triazolo[1,5- a]pyrazin-6-yl)benzonitrile | 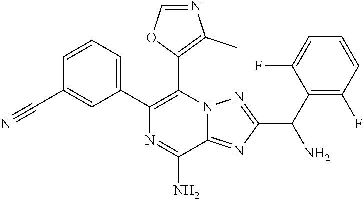 [SMILES: Cc1ncoc1-c1c(-c2cccc(C#N)c2)nc(N)c2nc(C(N)c3c(F)cccc3F)nn12] |
| 12 | 3-(8-amino-2-((2,6- difluorophenyl)(hydroxy)methyl)-5-(2,6- dimethylpyridin-4-yl)-[1,2,4]triazolo[1,5- a]pyrazin-6-yl)benzonitrile | 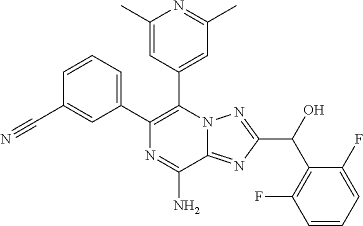 [SMILES: Cc1cc(-c2c(-c3cccc(C#N)c3)nc(N)c3nc(C(O)c4c(F)cccc4F)nn23)cc(C)n1] |
| 13 | 3-(4-amino-2-(pyridin-2-ylmethyl)-7- (pyrimidin-4-yl)-2H-[1,2,3]triazolo[4,5- c]pyridin-6-yl)benzonitrile | 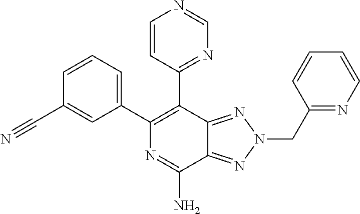 [SMILES: N#Cc1cccc(-c2nc(N)c3nn(Cc4ccccn4)nc3c2-c2ccncn2)c1] |
| 14 | 3-(4-amino-2-((3-fluoropyridin-2- yl)methyl)-7-(pyrimidin-4-yl)-2H- [1,2,3]triazolo[4,5-c]pyridin-6- yl)benzonitrile | 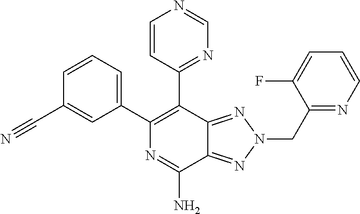 [SMILES: N#Cc1cccc(-c2nc(N)c3nn(Cc4ncccc4F)nc3c2-c2ccncn2)c1] |
| 15 | 3-(4-amino-2-((3-fluoropyridin-2- yl)methyl)-7-(pyridin-4-yl)-2H- [1,2,3]triazolo[4,5-c]pyridin-6- yl)benzonitrile | 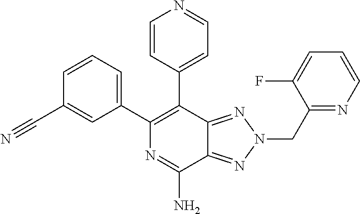 [SMILES: N#Cc1cccc(-c2nc(N)c3nn(Cc4ncccc4F)nc3c2-c2ccncc2)c1] |
| 16 | 3-(4-amino-7-(1-methyl-1H-pyrazol-5- yl)-2-(pyridin-2-ylmethyl)-2H- [1,2,3]triazolo[4,5-c]pyridin-6-yl)-2- fluorobenzonitrile | 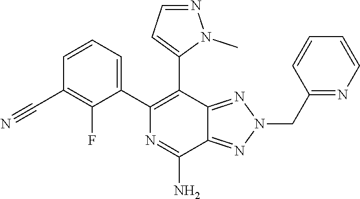 [SMILES: Cn1nccc1-c1c(-c2cccc(C#N)c2F)nc(N)c2nn(Cc3ccccn3)nc12] |
| 17 | 7-(1-((5-Chloropyridin-3-yl)methyl)-1H- pyrazol-4-yl)-3-methyl-9-pentyl-6,9- dihydro-5H-pyrrolo[3,2- d][1,2,4]triazolo[4,3-a]pyrimidin-5-one | 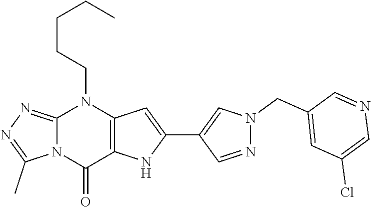 [SMILES: CCCCCn1c2cc(-c3cnn(Cc4cncc(Cl)c4)c3)[nH]c2c(=O)n2c(C)nnc12] |
| 18 | 3-Methyl-7-(1-((5-methylpyridin-3- yl)methyl)-1H-pyrazol-4-yl)-9-pentyl-6,9- dihydro-5H-pyrrolo[3,2- d][1,2,4]triazolo[4,3-a]pyrimidin-5-one | 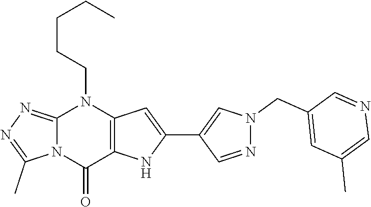 [SMILES: CCCCCn1c2cc(-c3cnn(Cc4cncc(C)c4)c3)[nH]c2c(=O)n2c(C)nnc12] |
| 19 | 3-Methyl-9-pentyl-7-(1-(thieno[3,2- b]pyridin-6-ylmethyl)-1H-pyrazol-4-yl)- 6,9-dihydro-5H-pyrrolo[3,2- d][1,2,4]triazolo[4,3-a]pyrimidin-5-one | 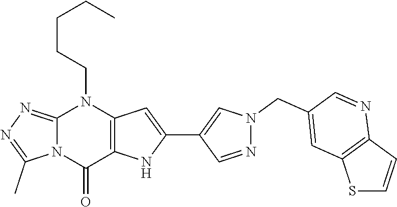 [SMILES: CCCCCn1c2cc(-c3cnn(Cc4cnc5ccsc5c4)c3)[nH]c2c(=O)n2c(C)nnc12] |
| 20 | 7-(1-((2-(2-(Dimethylamino)acetyl)- 1,2,3,4-tetrahydroisoquinolin-6- yl)methyl)-1H-pyrazol-4-yl)-3-methyl-9- pentyl-6,9-dihydro-5H-pyrrolo[3,2- d][1,2,4]triazolo[4,3-a]pyrimidin-5-one | 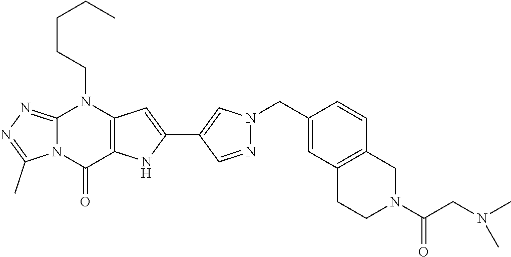 [SMILES: CCCCCn1c2cc(-c3cnn(Cc4ccc5c(c4)CCN(C(=O)CN(C)C)C5)c3)[nH]c2c(=O)n2c(C)nnc12] |
| 21A | 3-(2-((5-(1H-Pyrazol-1-yl)-2H-tetrazol-2- yl)methyl)-5-amino-8-(pyrimidin-4-yl)- [1,2,4]triazolo[1,5-c]pyrimidin-7- yl)benzonitrile | 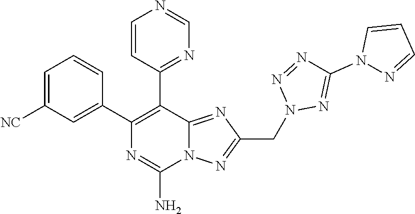 [SMILES: N#Cc1cccc(-c2nc(N)n3nc(Cn4nnc(-n5cccn5)n4)nc3c2-c2ccncn2)c1] |
| 21B | 3-(2-((5-(1H-Pyrazol-1-yl)-1H-tetrazol-1- yl)methyl)-5-amino-8-(pyrimidin-4-yl)- [1,2,4]triazolo[1,5-c]pyrimidin-7- yl)benzonitrile | 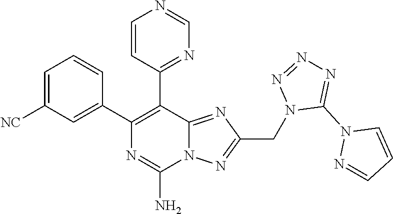 [SMILES: N#Cc1cccc(-c2nc(N)n3nc(Cn4nnnc4-n4cccn4)nc3c2-c2ccncn2)c1] |

In some embodiments, the inhibitor of A2A/A2B is a compound of Formula (I):

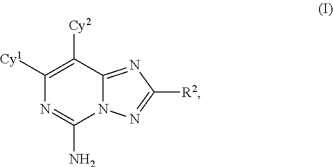

or a pharmaceutically acceptable salt thereof, wherein

Cy¹ is phenyl which is substituted by 1 or 2 substituents independently selected from halo and CN;

Cy² is 5-6 membered heteroaryl or 4-7 membered heterocycloalkyl, wherein the 5-6 membered heteroaryl or 4-7 membered heterocycloalkyl of Cy² are each optionally substituted with 1, 2, or 3 groups each independently selected from C₁₋₃ alkyl, C₁₋₃ alkoxy, NH₂, NH(C₁₋₃ alkyl) and N(C₁₋₃ alkyl)₂;

R² is selected from phenyl-C₁₋₃ alkyl-, C₃₋₇ cycloalkyl-C₁₋₃ alkyl-, (5-7 membered heteroaryl)-C₁₋₃ alkyl-, (4-7 membered heterocycloalkyl)-C₁₋₃ alkyl-, and OR^{a2}, wherein the phenyl-C₁₋₃ alkyl-, C₃₋₇ cycloalkyl-C₁₋₃ alkyl-, (5-7 membered heteroaryl)-C₁₋₃ alkyl-, and (4-7 membered heterocycloalkyl)-C₁₋₃ alkyl-of R² are each optionally substituted with 1, 2, or 3 independently selected R^{C} substituents;

R^{a2} is (5-7 membered heteroaryl)-C₁₋₃ alkyl-optionally substituted with 1 or 2 independently selected R^{C} substituents;

each R^{C} is independently selected from halo, C₁₋₆ alkyl, C₆ aryl, 5-7 membered heteroaryl, (4-7 membered heterocycloalkyl)-C₁₋₃ alkyl-, OR^{a4}, and NR^{e4}R^{d4}; and

each R^{a4}, R^{c4}, and R^{d4} are independently selected from H and C₁₋₆ alkyl.

In some embodiments of the compound of Formula (I), Cy² is pyrimidinyl.

In some embodiments of the compound of Formula (I), R² is selected from pyridin-2-ylmethyl, (2,6-difluorophenyl)(hydroxy)methyl, (5-(pyridin-2-yl)-1H-tetrazol-1-yl)methyl, (3-methylpyridin-2-yl)methoxy, and (5-(1H-Pyrazol-1-yl)-1H-tetrazol-1-yl)methyl.

In some embodiments, the compound of Formula (I), or a pharmaceutically acceptable salt thereof, is 3-(5-Amino-2-(pyridin-2-ylmethyl)-8-(pyrimidin-4-yl)-[1,2,4]triazolo[1,5-c]pyrimidin-7-yl)benzonitrile, or a pharmaceutically acceptable salt thereof (see Compound 1, Table 1).

In some embodiments, the compound of Formula (I), or a pharmaceutically acceptable salt thereof, is 3-(5-Amino-2-((2,6-difluorophenyl)(hydroxy)methyl)-8-(pyrimidin-4-yl)-[1,2,4]triazolo[1,5-c]pyrimidin-7-yl)benzonitrile, or a pharmaceutically acceptable salt thereof (see Compound 2, Table 1).

In some embodiments, the compound of Formula (I), or a pharmaceutically acceptable salt thereof, is 3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof (see Compound 3A, Table 1).

In some embodiments, the compound of Formula (I), or a pharmaceutically acceptable salt thereof, is 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile, or a pharmaceutically acceptable salt thereof (see Compound 3B, Table 1).

In some embodiments, the compound of Formula (I), or a pharmaceutically acceptable salt thereof, is 3-(5-Amino-2-((3-methylpyridin-2-yl)methoxy)-8-(pyrimidin-4-yl)-[1,2,4]triazolo[1,5-c]pyrimidin-7-yl)benzonitrile, or a pharmaceutically acceptable salt thereof (see Compound 4, Table 1).

In some embodiments, the compound of Formula (I), or a pharmaceutically acceptable salt thereof, is 3-(2-((5-(1H-pyrazol-1-yl)-2H-tetrazol-2-yl)methyl)-5-amino-8-(pyrimidin-4-yl)-[1,2,4]triazolo[1,5-c]pyrimidin-7-yl)benzonitrile, or a pharmaceutically acceptable salt thereof (see Compound 21A, Table 1).

In some embodiments, the compound of Formula (I), or a pharmaceutically acceptable salt thereof, is 3-(2-((5-(1H-Pyrazol-1-yl)-1H-tetrazol-1-yl)methyl)-5-amino-8-(pyrimidin-4-yl)-[1,2,4]triazolo[1,5-c]pyrimidin-7-yl)benzonitrile, or a pharmaceutically acceptable salt thereof (see Compound 21B, Table 1).

In some embodiments, the inhibitor of A2A/A2B is selected from:

3-(5-Amino-2-(pyridin-2-ylmethyl)-8-(pyrimidin-4-yl)-[1,2,4]triazolo[1,5-c]pyrimidin-7-yl)benzonitrile, or a pharmaceutically acceptable salt thereof;
3-(5-Amino-2-((2,6-difluorophenyl)(hydroxy)methyl)-8-(pyrimidin-4-yl)-[1,2,4]triazolo[1,5-c]pyrimidin-7-yl)benzonitrile, or a pharmaceutically acceptable salt thereof;
3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile, or a pharmaceutically acceptable salt thereof;
3-(5-Amino-2-((3-methylpyridin-2-yl)methoxy)-8-(pyrimidin-4-yl)-[1,2,4]triazolo[1,5-c]pyrimidin-7-yl)benzonitrile, or a pharmaceutically acceptable salt thereof; and
3-(2-((5-(1H-Pyrazol-1-yl)-1H-tetrazol-1-yl)methyl)-5-amino-8-(pyrimidin-4-yl)-[1,2,4]triazolo[1,5-c]pyrimidin-7-yl)benzonitrile, or a pharmaceutically acceptable salt thereof.

The synthesis and characterization of compounds of Formula (I) can be found in WO2019/168847 and U.S. 62/891,685, both of which are hereby incorporated by reference in their entireties.

In some embodiments, the inhibitor of A2A/A2B is a compound of Formula (II):

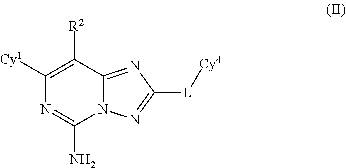

or a pharmaceutically acceptable salt thereof, wherein

R² is selected from H and CN;

Cy¹ is phenyl which is substituted by 1 or 2 substituents independently selected from halo and CN;

L is C₁₋₃ alkylene, wherein said alkylene is optionally substituted with 1, 2, or 3 independently selected R^{8D} substituents;

Cy⁴ is selected from phenyl, cyclohexyl, pyridyl, pyrrolidinonyl, and imidazolyl, wherein the phenyl, cyclohexyl, pyridyl, pyrrolidinonyl, and imidazolyl are each optionally substituted with 1, 2, or 3 substituents independently selected from R^{8D} and R⁸.

each R⁸ is independently selected from halo, C₁₋₆ alkyl, C₁₋₆ haloalkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl, wherein the C₁₋₆ alkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl of R⁸ are each optionally substituted with 1, 2, or 3 independently selected R^{8A} substituents;

each R^{8A} is independently selected from halo, C₁₋₆ alkyl, 5-6 membered heteroaryl, 4-7 membered heterocycloalkyl, CN, OR^{a81}, and NR^{c81}R^{d81}, wherein the C₁₋₃ alkyl, 5-6 membered heteroaryl, and 4-7 membered heterocycloalkyl of R^{8A} are each optionally substituted with 1, 2, or 3 independently selected R^{8B} substituents;

each R^{a81}, R^{c81}, and R^{d81} is independently selected from H, C₁₋₆ alkyl, and 4-7 membered heterocycloalkyl, wherein the C₁₋₆ alkyl and 4-7 membered heterocycloalkyl of R^{a81}, R^{c81}, and R^{d81} are each optionally substituted with 1, 2, or 3 independently selected R^{8B} substituents;

each R^{8B} is independently selected from halo and C₁₋₃ alkyl; and

each R^{8D} is independently selected from OH, CN, halo, C₁₋₆ alkyl, and C₁₋₆ haloalkyl.

In some embodiments, the compound of Formula (II), or a pharmaceutically acceptable salt thereof, is 3-(5-Amino-2-(hydroxy(phenyl)methyl)-[1,2,4]triazolo[1,5-c]pyrimidin-7-yl)benzonitrile, or a pharmaceutically acceptable salt thereof (see Compound 5, Table 1).

In some embodiments, the compound of Formula (II), or a pharmaceutically acceptable salt thereof, is 3-(5-Amino-2-((2,6-difluorophenyl)(hydroxy)methyl)-[1,2,4]triazolo[1,5-c]pyrimidin-7-yl)-2-fluorobenzonitrile, or a pharmaceutically acceptable salt thereof (see Compound 6, Table 1).

In some embodiments, the compound of Formula (II), or a pharmaceutically acceptable salt thereof, is 5-Amino-7-(3-cyano-2-fluorophenyl)-2-((2,6-difluorophenyl)(hydroxy)methyl)-[1,2,4]triazolo[1,5-c]pyrimidine-8-carbonitrile, or a pharmaceutically acceptable salt thereof (see Compound 7, Table 1).

In some embodiments, the compound of Formula (II), or a pharmaceutically acceptable salt thereof, is 3-(5-Amino-2-((2-fluoro-6-(((1-methyl-2-oxopyrrolidin-3-yl)amino)methyl)phenyl)(hydroxy)methyl)-[1,2,4]triazolo[1,5-c]pyrimidin-7-yl)-2-fluorobenzonitrile, or a pharmaceutically acceptable salt thereof (see Compound 8, Table 1).

The synthesis and characterization of compounds of Formula (II) can be found in WO2019/222677, which is hereby incorporated by reference in its entirety.

In some embodiments, the inhibitor of A2A/A2B is a compound of Formula (III):

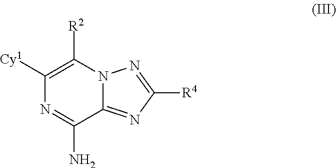

or a pharmaceutically acceptable salt thereof, wherein

Cy¹ is phenyl which is substituted by 1 or 2 substituents independently selected from halo and CN;

R² is selected from 5-6 membered heteroaryl and 4-7 membered heterocycloalkyl, wherein the 5-6 membered heteroaryl and 4-7 membered heterocycloalkyl of R² are each optionally substituted with 1, 2, or 3 independently selected R^{2A} substituents;

each R^{2A} is independently selected from D, halo, C₁₋₆ alkyl, and C₁₋₆ haloalkyl;

R⁴ is selected from phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl wherein the phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl-of R⁴ are each optionally substituted with 1, 2, or 3 independently selected R^{4A} substituents;

each R^{4A} is independently selected from halo, C₁₋₆ alkyl, C₁₋₆ haloalkyl, CN, OR^{a41}, and NR^{c41}R^{d41}; and

each R^{a41}, R^{c41}, and R^{d41} is independently selected from H and C₁₋₆ alkyl.

In some embodiments, the compound of Formula (III), or a pharmaceutically acceptable salt thereof, is 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof (see Compound 9, Table 1).

In some embodiments, the compound of Formula (III), or a pharmaceutically acceptable salt thereof, is 3-(8-Amino-2-((2,6-difluorophenyl)(hydroxy)methyl)-5-(pyrimidin-4-yl)-[1,2,4]triazolo[1,5-a]pyrazin-6-yl)benzonitrile, or a pharmaceutically acceptable salt thereof (See Compound 10, Table 1).

In some embodiments, the compound of Formula (III), or a pharmaceutically acceptable salt thereof, is 3-(8-amino-2-(amino(2,6-difluorophenyl)methyl)-5-(4-methyloxazol-5-yl)-[1,2,4]triazolo[1,5-a]pyrazin-6-yl)benzonitrile, or a pharmaceutically acceptable salt thereof (see Compound 11, Table 1).

In some embodiments, the compound of Formula (III), or a pharmaceutically acceptable salt thereof, is 3-(8-amino-2-((2,6-difluorophenyl)(hydroxy)methyl)-5-(2,6-dimethylpyridin-4-yl)-[1,2,4]triazolo[1,5-a]pyrazin-6-yl)benzonitrile, or a pharmaceutically acceptable salt thereof (see Compound 12, Table 1).

The synthesis and characterization of compounds of Formula (III) can be found in PCT/US2019/040496, which is hereby incorporated by reference in its entirety.

In some embodiments, the inhibitor of A2A/A2B is a compound of Formula (IV):

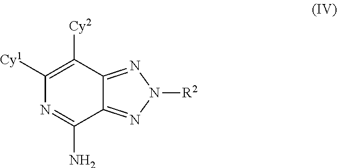

or a pharmaceutically acceptable salt thereof, wherein

Cy¹ is phenyl which is substituted by 1 or 2 substituents independently selected from halo and CN;

Cy² is selected from 5-6 membered heteroaryl and 4-7 membered heterocycloalkyl, wherein the 5-6 membered heteroaryl and 4-7 membered heterocycloalkyl of Cy² are each optionally substituted with 1, 2, or 3 independently selected R⁶ substituents;

each R⁶ is independently selected from halo, C₁₋₆ alkyl, and C₁₋₆ haloalkyl;

R² is phenyl-C₁₋₃ alkyl- or (5-6 membered heteroaryl)-C₁₋₃ alkyl-, wherein the phenyl-C₁₋₃ alkyl- and (5-6 membered heteroaryl)-C₁₋₃ alkyl-of R² are each optionally substituted with 1, 2, or 3 independently selected R^{2A} substituents; and

each R^{2A} is independently selected from halo, C₁₋₆ alkyl, and C₁₋₆ haloalkyl.

In some embodiments, the compound of Formula (IV), or a pharmaceutically acceptable salt thereof, is 3-(4-amino-2-(pyridin-2-ylmethyl)-7-(pyrimidin-4-yl)-2H-[1,2,3]triazolo[4,5-c]pyridin-6-yl)benzonitrile, or a pharmaceutically acceptable salt thereof (see Compound 13, Table 1).

In some embodiments, the compound of Formula (IV), or a pharmaceutically acceptable salt thereof, is 3-(4-amino-2-((3-fluoropyridin-2-yl)methyl)-7-(pyrimidin-4-yl)-2H-[1,2,3]triazolo[4,5-c]pyridin-6-yl)benzonitrile, or a pharmaceutically acceptable salt thereof (see Compound 14, Table 1).

In some embodiments, the compound of Formula (IV), or a pharmaceutically acceptable salt thereof, is 3-(4-amino-2-((3-fluoropyridin-2-yl)methyl)-7-(pyridin-4-yl)-2H-[1,2,3]triazolo[4,5-c]pyridin-6-yl)benzonitrile, or a pharmaceutically acceptable salt thereof (see Compound 15, Table 1).

In come embodiments, the compound of Formula (IV), or a pharmaceutically acceptable salt thereof, is 3-(4-amino-7-(1-methyl-1H-pyrazol-5-yl)-2-(pyridin-2-ylmethyl)-2H-[1,2,3]triazolo[4,5-c]pyridin-6-yl)-2-fluorobenzonitrile, or a pharmaceutically acceptable salt thereof (see Compound 16, Table 1).

The synthesis and characterization of compounds of Formula (IV) can be found in U.S. 62/798,180, which is hereby incorporated by reference in its entirety.

In some embodiments, the inhibitor of A2A/A2B is a compound of Formula (V):

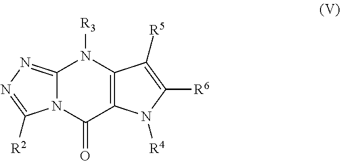

or a pharmaceutically acceptable salt thereof, wherein

R² is selected from H, D, halo, C₁₋₆ alkyl and C₁₋₆ haloalkyl;

R³ is selected from H and C₁₋₆ alkyl;

R⁴ is selected from H and C₁₋₆ alkyl;

R^{s} is selected from H, halo, CN, C₁₋₆ alkyl;

R⁶ is selected from phenyl, C₃₋₇ cycloalkyl, 5-7 membered heteroaryl, and 4-7 membered heterocycloalkyl wherein said phenyl, C₃₋₇ cycloalkyl, 5-7 membered heteroaryl, and 4-7 membered heterocycloalkyl of R⁶ are optionally substituted by 1, 2, or 3 independently selected R^{A} substituents;

each R^{A} is independently selected from (5-10 membered heteroaryl)-C₁₋₃ alkyl- and (4-10 membered heterocycloalkyl)-C₁₋₃ alkyl-, wherein the (5-10 membered heteroaryl)-C₁₋₃ alkyl- and (4-10 membered heterocycloalkyl)-C₁₋₃ alkyl-of R^{A} are each optionally substituted with 1 or 2 independently selected R^{B} substituents;

each R^{B} is independently selected from halo, C₁₋₆ alkyl, and C(O)R^{b26};

R^{b26} is independently selected from H and C₁₋₃ alkyl, wherein the C₁₋₃ alkyl of R^{b26} is optionally substituted with 1 or 2 independently selected R^{C} substituents

each R^{C} is independently selected from halo, C₁₋₆ alkyl, CN, OR^{a36}, and NR^{c36}R^{d36}; and

each R^{a36}, R^{c36}, and R^{d36} is independently selected from H and C₁₋₆ alkyl.

In some embodiments, the compound of Formula (V), or a pharmaceutically acceptable salt thereof, is 7-(1-((5-Chloropyridin-3-yl)methyl)-1H-pyrazol-4-yl)-3-methyl-9-pentyl-6,9-dihydro-5H-pyrrolo[3,2-d][1,2,4]triazolo[4,3-a]pyrimidin-5-one, or a pharmaceutically acceptable salt thereof (see Compound 17, Table 1).

In some embodiments, the compound of Formula (V), or a pharmaceutically acceptable salt thereof, is 3-Methyl-7-(1-((5-methylpyridin-3-yl)methyl)-1H-pyrazol-4-yl)-9-pentyl-6,9-dihydro-5H-pyrrolo[3,2-d][1,2,4]triazolo[4,3-a]pyrimidin-5-one, or a pharmaceutically acceptable salt thereof (see Compound 18, Table 1).

In some embodiments, the compound of Formula (V), or a pharmaceutically acceptable salt thereof, is 3-Methyl-9-pentyl-7-(1-(thieno[3,2-b]pyridin-6-ylmethyl)-1H-pyrazol-4-yl)-6,9-dihydro-5H-pyrrolo[3,2-d][1,2,4]triazolo[4,3-a]pyrimidin-5-one, or a pharmaceutically acceptable salt thereof (see Compound 19, Table 1).

In some embodiments, the compound of Formula (V), or a pharmaceutically acceptable salt thereof, is 7-(1-((2-(2-(Dimethylamino)acetyl)-1,2,3,4-tetrahydroisoquinolin-6-yl)methyl)-1H-pyrazol-4-yl)-3-methyl-9-pentyl-6,9-dihydro-5H-pyrrolo[3,2-d][1,2,4]triazolo[4,3-a]pyrimidin-5-one, or a pharmaceutically acceptable salt thereof (see Compound 20, Table 1).

The synthesis and characterization of compounds of Formula (V) can be found in US-2019-0337957, which is hereby incorporated by reference in its entirety.

### PD-1/PD-L1 Inhibitors

The immune system plays an important role in controlling and eradicating diseases such as cancer. However, cancer cells often develop strategies to evade or to suppress the immune system in order to favor their growth. One such mechanism is altering the expression of co-stimulatory and co-inhibitory molecules expressed on immune cells (Postow et al., J. Clinical Oncology 2015, 1-9). Blocking the signaling of an inhibitory immune checkpoint, such as PD-1, has proven to be a promising and effective treatment modality.

Programmed Death-1 (“PD-1,” also known as “CD279”) is an approximately 31 kD type I membrane protein member of the extended CD28/CTLA-4 family of T-cell regulators that broadly negatively regulates immune responses (Ishida, Y. et al. (1992) EMBO J. 11:3887-3895; United States Patent Publication No. 2007/0202100; 2008/0311117; and 2009/00110667; U.S. Pat. Nos. 6,808,710; 7,101,550; 7,488,802; 7,635,757; and 7,722,868; PCT Publication No. WO 01/14557).

PD-1 is expressed on activated T-cells, B-cells, and monocytes (Agata, Y. et al. (1996) Int. Immunol. 8(5):765-772; Yamazaki, T. et al. (2002) J. Immunol. 169:5538-5545) and at low levels in natural killer (NK) T-cells (Nishimura, H. et al. (2000) J. Exp. Med. 191:891-898; Martin-Orozco, N. et al. (2007) Semin. Cancer Biol. 17(4):288-298).

The extracellular region of PD-1 consists of a single immunoglobulin (Ig)V domain with 23% identity to the equivalent domain in CTLA-4 (Martin-Orozco, N. et al. (2007) Semin. Cancer Biol. 17(4):288-298). The extracellular IgV domain is followed by a transmembrane region and an intracellular tail. The intracellular tail contains two phosphorylation sites located in an immunoreceptor tyrosine-based inhibitory motif and an immunoreceptor tyrosine-based switch motif, which suggests that PD-1 negatively regulates TCR signals (Ishida, Y. et al. (1992) EMBO J. 11:3887-3895; Blank, C. et al. (2006) Immunol. Immunother. 56(5):739-745).

PD-1 mediates its inhibition of the immune system by binding to B7-H1 and B7-DC (Flies, D. B. et al. (2007) J. Immunother. 30(3):251-260; U.S. Pat. Nos. 6,803,192; 7,794,710; United States Patent Application Publication Nos. 2005/0059051; 2009/0055944; 2009/0274666; 2009/0313687; PCT Publication Nos. WO 01/39722; WO 02/086083).

The amino acid sequence of the human PD-1 protein (Genbank Accession No. NP_005009) is:

(SEQ ID NO: 1)
MQIPQAPWPVVWAVLQLGWRPGWFLDSPDRPWNPPTFSPALLVVT
EGDNATFTCSFSNTSESFVLNWYRMSPSNQTDKLAAFPEDRSQPG
QDCRFRVTQLPNGRDFHMSVVRARRNDSGTYLCGAISLAPKAQIK
ESLRAELRVTERRAEVPTAHPSPSPRPAGQFQTLVVGVVGGLLGS
LVLLVWVLAVICSRAARGTIGARRTGQPLKEDPSAVPVFSVDYGE
LDFQWREKTPEPPVPCVPEQTEYATIVFPSGMGTSSPARRGSADG
PRSAQPLRPEDGHCSWPL.

PD-1 has two ligands, PD-LI and PD-L2 (Parry et al, Mol Cell Biol 2005, 9543-9553; Latchman et al, Nat Immunol 2001, 2, 261-268), and they differ in their expression patterns. PD-L1 protein is upregulated on macrophages and dendritic cells in response to lipopolysaccharide and GM-CSF treatment, and on T cells and B cells upon T cell receptor and B cell receptor signaling. PD-L1 is also highly expressed on almost all tumor cells, and the expression is further increased after IFN-7 treatment (Iwai et al, PNAS 2002, 99(19):12293-7; Blank et al, Cancer Res 2004, 64(3):1140-5). In fact, tumor PD-L1 expression status has been shown to be prognostic in multiple tumor types (Wang et al, Eur J Surg Oncol 2015; Huang et al, Oncol Rep 2015; Sabatier et al, Oncotarget 2015, 6(7): 5449-5464). PD-L2 expression, in contrast, is more restricted and is expressed mainly by dendritic cells (Nakae et al, J Immunol 2006, 177:566-73). Ligation of PD-1 with its ligands PD-L1 and PD-L2 on T cells delivers a signal that inhibits IL-2 and IFN-γ production, as well as cell proliferation induced upon T cell receptor activation (Carter et al, Eur J Immunol 2002, 32(3):634-43; Freeman et al, J Exp Med 2000, 192(7):1027-34). The mechanism involves recruitment of SHP-2 or SHP-1 phosphatases to inhibit T cell receptor signaling such as Syk and Lek phosphorylation (Sharpe et al, Nat Immunol 2007, 8, 239-245). Activation of the PD-1 signaling axis also attenuates PKC-θ activation loop phosphorylation, which is necessary for the activation of NF-κB and APi pathways, and for cytokine production such as IL-2, IFN-γ and TNF (Sharpe et al, Nat Immunol 2007, 8, 239-245; Carter et al, Eur J Immunol 2002, 32(3):634-43; Freeman et al, J Exp Med 2000, 192(7):1027-34).

Several lines of evidence from preclinical animal studies indicate that PD-1 and its ligands negatively regulate immune responses. PD-1-deficient mice have been shown to develop lupus-like glomerulonephritis and dilated cardiomyopathy (Nishimura et al, Immunity 1999, 11:141-151; Nishimura et al., Science 2001, 291:319-322). Using an LCMV model of chronic infection, it has been shown that PD-1/PD-LI interaction inhibits activation, expansion and acquisition of effector functions of virus-specific CD8 T cells (Barber et al., Nature 2006, 439, 682-7). Together, these data support the development of a therapeutic approach to block the PD-1-mediated inhibitory signaling cascade in order to augment or “rescue” T cell response. Accordingly, there is a need for new methods of blocking PD-1/PD-L1 protein/protein interaction, and thereby treating cancer in a subject.

In some embodiments, the inhibitor of PD-1/PD-Li is a compound selected from nivolumab (OPDIVO®, BMS-936558, MDX1106, or MK-34775), pembrolizumab (KEYTRUDA®, MK-3475, SCH-900475, lambrolizumab, CAS Reg. No. 1374853-91-4), atezolizumab (Tecentriq®, CAS Reg. No. 1380723-44-3), durvalumab, avelumab (Bavencio®), cemiplimab, AMP-224, AMP-514/MEDI-0680, atezolizumab, avelumab, BGB-A317, BMS936559, durvalumab, JTX-4014, SHR-1210, pidilizumab (CT-011), REGN2810, BGB-108, BGB-A317, SHR-1210 (HR-301210, SHR1210, or SHR-1210), BMS-936559, MPDL3280A, MEDI4736, MSB0010718C, MDX1105-01, and one or more of the PD-1/PD-L1 blocking agents described in U.S. Pat. Nos. 7,488,802, 7,943,743, 8,008,449, 8,168,757, 8,217,149, or Pub. Nos. WO 03042402, WO 2008/156712, WO 2010/089411, WO 2010/036959, WO 2011/066342, WO 2011/159877, WO 2011/082400, WO 2011/161699, WO 2017/070089, WO 2017/087777, WO 2017/106634, WO 2017/112730, WO 2017/192961, WO 2017/205464, WO 2017/222976, WO 2018/013789, WO 2018/04478, WO 2018/119236, WO 2018/119266, WO 2018/119221, WO 2018/119286, WO 2018/119263, WO 2018/119224, WO 2019/191707, and WO 2019/217821, and any combinations thereof. The disclosure of each of the preceding patents, applications, and publications is incorporated herein by reference in its entirety.

In some embodiments, the inhibitor of PD-1/PD-Li is selected from a compound as disclosed in WO 2018/119266 such as, e.g.,
(S)-1-((7-chloro-2-(2′-chloro-3′-(5-(((2-hydroxyethyl)amino)methyl)picolinamido)-2-methyl-[i1,1′-biphenyl]-3-yl)benzo[d]oxazol-5-yl)methyl)piperidine-2-carboxylic acid, or a pharmaceutically acceptable salt thereof;
(S)-1-((7-chloro-2-(3′-(7-chloro-5-(((S)-3-hydroxypyrrolidin-1-yl)methyl)benzo[d]oxazol-2-yl)-2,2′-dimethylbiphenyl-3-yl)benzo[d]oxazol-5-yl)methyl)pyrrolidine-3-carboxylic acid, or a pharmaceutically acceptable salt thereof;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof;
(S)-1-((2-(2′-chloro-3′-(1,5-dimethyl-4,5,6,7-tetrahydro-1H-imidazo[4,5-c]pyridine-2-carboxamido)-2-methylbiphenyl-3-yl)-7-cyanobenzo[d]oxazol-5-yl)methyl)pyrrolidine-3-carboxylic acid, or a pharmaceutically acceptable salt thereof;
(R)-1-((7-cyano-2-(2,2′-dimethyl-3′-(4,5,6,7-tetrahydrothiazolo[5,4-c]pyridin-2-yl)biphenyl-3-yl)benzo[d]oxazol-5-yl)methyl)pyrrolidine-3-carboxylic acid, or a pharmaceutically acceptable salt thereof;
(R)-1-((7-cyano-2-(3′-(5-(2-(dimethylamino)acetyl)-5,6-dihydro-4H-pyrrolo[3,4-d]thiazol-2-yl)-2,2′-dimethylbiphenyl-3-yl)benzol[d]oxazol-5-yl)methyl)pyrrolidine-3-carboxylic acid, or a pharmaceutically acceptable salt thereof; and
1-((7-cyano-2-(3′-(5-(2-(dimethylamino)acetyl)-5,6-dihydro-4H-pyrrolo[3,4-d]thiazol-2-yl)-2,2′-dimethylbiphenyl-3-yl)benzol[d]oxazol-5-yl)methyl)piperidine-4-carboxylic acid, or a pharmaceutically acceptable salt thereof.

In some embodiments, the inhibitor of PD-1/PD-Li is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof.
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof is also referred to herein as Compound Y. The synthesis and characterization of Compound Y is disclosed in WO 2018/119266, which is hereby incorporated by reference in its entirety.

In some embodiments, the inhibitor of PD-1/PD-Li is selected from:
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid hydrobromic acid salt;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid oxalic acid salt;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid hydrochloric acid salt;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid L-tartaric acid salt;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid malonic acid salt; and
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid phosphoric acid salt.

In some embodiments, the inhibitor of PD-1/PD-Li is selected from a compound disclosed in WO 2018/119224 such as, e.g.,
(S)-1-((2-(2′-chloro-3′-(1,5-dimethyl-4,5,6,7-tetrahydro-1H-imidazo[4,5-c]pyridine-2-carboxamido)-2-methylbiphenyl-3-yl)-7-cyanobenzo[d]oxazol-5-yl)methyl)pyrrolidine-3-carboxylic acid, or a pharmaceutically acceptable salt thereof;
(R)-1-((2-(2′-chloro-3′-(6-isopropyl-4,5,6,7-tetrahydro-2H-pyrazolo[3,4-c]pyridin-2-yl)-2-methylbiphenyl-3-yl)-7-cyanobenzo[d]oxazol-5-yl)methyl)pyrrolidine-3-carboxylic acid, or a pharmaceutically acceptable salt thereof;
(S)—N-(2-chloro-3′-(5-(2-hydroxypropyl)-1-methyl-4,5,6,7-tetrahydro-1H-imidazo[4,5-c]pyridine-2-carboxamido)-2′-methylbiphenyl-3-yl)-5-isopropyl-1-methyl-4,5,6,7-tetrahydro-1H-imidazo[4,5-c]pyridine-2-carboxamide, or a pharmaceutically acceptable salt thereof;
cis-4-((2-((2,2′-dichloro-3′-(1-methyl-5-(tetrahydro-2H-pyran-4-yl)-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)methyl)cyclohexane-1-carboxylic acid, or a pharmaceutically acceptable salt thereof;
trans-4-(2-(2-((2,2′-dichloro-3′-(5-(2-hydroxyethyl)-1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)cyclohexane-1-carboxylic acid, or a pharmaceutically acceptable salt thereof;
trans-4-(2-(2-((2-chloro-2′-methyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)cyclohexane-1-carboxylic acid, or a pharmaceutically acceptable salt thereof; and
cis-4-((2-(2-chloro-3′-(5-(2-(ethyl(methyl)amino)acetyl)-5,6-dihydro-4H-pyrrolo[3,4-d]thiazol-2-yl)-2′-methylbiphenyl-3-ylcarbamoyl)-1-methyl-6,7-dihydro-1H-imidazo[4,5-c]pyridin-5(4H)-yl)methyl)cyclohexane-1-carboxylic acid, or a pharmaceutically acceptable salt thereof.

In some embodiments, the inhibitor of PD-1/PD-Li is selected from a compound disclosed in WO 2019/191707 such as, e.g.,
(R)-1-((7-cyano-2-(3′-(7-((3-hydroxypyrrolidin-1-yl)methyl)-2-methylpyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof;
(R)-1-((7-cyano-2-(3′-(7-(((S)-1-hydroxypropan-2-ylamino)methyl)-2-methylpyrido[3,2-d]pyrimidin-4-ylamino)-2,2′-dimethylbiphenyl-3-yl)benzo[d]oxazol-5-yl)methyl)pyrrolidine-3-carboxylic acid, or a pharmaceutically acceptable salt thereof;
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof;
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)-N,N-dimethylpiperidine-4-carboxamide, or a pharmaceutically acceptable salt thereof;
(R)-1-((7-cyano-2-(3′-(2-cyclopropyl-7-(((R)-3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)pyrrolidine-3-carboxylic acid, or a pharmaceutically acceptable salt thereof; and
(R)-1-((7-cyano-2-(3′-(3-(((R)-3-hydroxypyrrolidin-1-yl)methyl)-6-methyl-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof.

In some embodiments, the inhibitor of PD-1/PD-Li is selected from a compound disclosed in WO 2019/217821 such as, e.g.,
4-(2-(2-((2,2′-dichloro-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof;
4-(2-(2-((3′-(5-((1H-pyrazol-3-yl)methyl)-1-methyl-4,5,6,7-tetrahydro-1H-imidazo[4,5-c]pyridine-2-carboxamido)-2,2′-dichloro-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof;
(R)-4-(2-(2-((2,2′-dichloro-3′-(5-(2-hydroxypropyl)-1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof;
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid), or a pharmaceutically acceptable salt thereof;
4-(2-(2-((2-chloro-2′-methyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof;
4-(2-(2-((2,2′-dimethyl-3′-(1-methyl-4,5,6,7-tetrahydro-1H-imidazo[4,5-c]pyridine-2-carboxamido)-1[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof; and
4-(2-(2-((3′-(5-(trans-4-carboxy-4-methylcyclohexyl)-1-methyl-4,5,6,7-tetrahydro-1H-imidazo[4,5-c]pyridine-2-carboxamido)-2,2′-dichloro-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof.

In some embodiments, the inhibitor of PD-1/PD-Li is pembrolizumab.

In some embodiments, the inhibitor of PD-1/PD-Li is nivolumab.

In some embodiments, the inhibitor of PD-1/PD-Li is atezolizumab.

In some embodiments, the inhibitor of PD-1/PD-Li is ANTIBODY X. As used herein, the ANTIBODY X is a humanized IgG4 monoclonal antibody that binds to human PD-1. See hPD-1 mAb 7(1.2) in WO2017019846, which is incorporated herein by reference in its entirety. The amino acid sequences of the mature ANTIBODY X heavy and light chains are shown below. Complementarity-determining regions (CDRs) 1, 2, and 3 of the variable heavy (VH) domain and the variable light (VL) domain are shown in that order from N to the C-terminus of the mature VL and VH sequences and are both underlined and bolded. An antibody consisting of the mature heavy chain (SEQ ID NO:2) and the mature light chain (SEQ ID NO:3) listed below is termed ANTIBODY X.

Mature ANTIBODY X heavy chain (HC)
(SEQ ID NO: 2)
QVQLVQSGAEVKKPGASVKVSCKASGYSFTSYWMNWVRQAPGQGL
EWIGVIHPSDSETWLDQKFKDRVTITVDKSTSTAYMELSSLRSED
TAVYYCAREHYGTSPFAYWGQGTLVTVSSASTKGPSVFPLAPCSR
STSESTAALGCLVKDYFPEPVTVSWNSGALTSGVHTFPAVLQSSG
LYSLSSVVTVPSSSLGTKTYTCNVDHKPSNTKVDKRVESKYGPPC
PPCPAPEFLGGPSVFLFPPKPKDTLMISRTPEVTCVVVDVSQEDP
EVQFNWYVDGVEVHNAKTKPREEQFNSTYRVVSVLTVLHQDWLNG
KEYKCKVSNKGLPSSIEKTISKAKGQPREPQVYTLPPSQEEMTKN
QVSLTCLVKGFYPSDIAVEWESNGQPENNYKTTPPVLDSDGSFFL
YSRLTVDKSRWQEGNVFSCSVMHEALHNHYTQKSLSLSLG
Mature ANTIBODY X light chain (LC)
(SEQ ID NO: 3)
EIVLTQSPATLSLSPGERATLSCRASESVDNYGMSFMNWFQQKPG
QPPKLLIHAASNQGSGVPSRFSGSGSGTDFTLTISSLEPEDFAVY
FCQQSKEVPYTFGGGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC

The variable heavy (VH) domain of ANTIBODY X has the following amino acid sequence:

(SEQ ID NO: 4)
QVQLVQSGAEVKKPGASVKVSCKASGYSFTSYWMNWVRQAPGQGL
EWIGVIHPSDSETWLDQKFKDRVTITVDKSTSTAYMELSSLRSED
TAVYYCAREHYGTSPFAYWGQGTLVTVSS

The variable light (VL) domain of ANTIBODY X has the following amino acid sequence:

(SEQ ID NO: 5)
EIVLTQSPATLSLSPGERATLSCRASESVDNYGMSFMNWFQQKPG
QPPKLLIHAASNQGSGVPSRFSGSGSGTDFTLTISSLEPEDFAVY
FCQQSKEVPYTFGGGTKVEIK

The amino acid sequences of the VH CDRs of ANTIBODY X are listed below:

VH CDR1:
(SEQ ID NO: 6)
SYWMN;
VH CDR2:
(SEQ ID NO: 7)
VIHPSDSETWLDQKFKD;
VH CDR3:
(SEQ ID NO: 8)
EHYGTSPFAY

The amino acid sequences of VL CDRs of ANTIBODY X are listed below:

VL CDR1:
(SEQ ID NO: 9)
RASESVDNYGMSFMNW;
VL CDR2:
(SEQ ID NO: 10)
AASNQGS;
and
VL CDR3:
(SEQ ID NO: 11)
QQSKEVPYT.

As used herein, “QD” is taken to mean a dosage administered to the subject once-daily. “QOD” is taken to mean a dosage administered to the subject once, every other day. “QW” is taken to mean a dosage administered to the subject once-weekly. “Q2W” is taken to mean a dosage administered to the subject once, every other week. “Q3W” is taken to mean a dosage administered to the subject once, every three weeks. “Q4W” is taken to mean a dosage administered to the subject once, every four weeks.

As used herein, “about” when referring to a measurable value such as an amount, a dosage, a temporal duration, and the like, is meant to encompass variations of ±10%. In certain embodiments, “about” can include variations of ±5%, ±1%, or ±0.1% from the specified value and any variations there between, as such variations are appropriate to perform the disclosed methods.

In some embodiments, the compound disclosed herein is the (S)-enantiomer of the compound, or a pharmaceutically acceptable salt thereof. In some embodiments, the compound is the (R)-enantiomer of the compound, or a pharmaceutically acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.” Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 5 to 10 carbon atoms. In some embodiments, the aryl group is phenyl or naphthyl. In some embodiments, the aryl is phenyl (i.e., C₆ aryl).

As used herein, “halo” or “halogen” refers to F, Cl, Br, or I. In some embodiments, a halo is F, Cl, or Br. In some embodiments, a halo is F or Cl. In some embodiments, a halo is F. In some embodiments, a halo is Cl.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, S, and B. In some embodiments, the heteroaryl is a 5-6 monocyclic heteroaryl having 1 or 2 heteroatom ring members independently selected from N, O, and S. In some embodiments, the heteroaryl group contains 3 to 10, 4 to 10, 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1, 2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, indazolyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups. Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring.

In some embodiments, the heterocycloalkyl group contains 3 to 10 ring-forming atoms, 4 to 10 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10 membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, diazaspiro[3.3]heptanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 3-oxopiperazinyl, oxo-pyrrolidinyl, oxo-pyridinyl and the like. For example, heterocycloalkyl groups include the following groups (with and without N-methyl substitution):

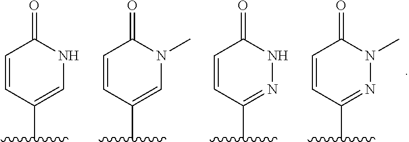

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., =O) as a divalent substituent, forming a carbonyl group when attached to a carbon (e.g., C═O or C(O)), or attached to a nitrogen or sulfur heteroatom forming a nitroso, sulfinyl or sulfonyl group.

As used herein, the term “independently selected from” means that each occurrence of a variable or substituent are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C=N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula (I), (II), etc.) provided herein include stereoisomers of the compounds.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide-imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

Compounds described herein, including salts thereof, can be prepared using known organic synthesis techniques and can be synthesized according to any of numerous possible synthetic routes.

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) (“Preparative LC-MS Purification: Improved Compound Specific Method Optimization” Karl F. Blom, et al. J. Combi. Chem. 2004, 6(6), 874-883, which is incorporated herein by reference in its entirety) and normal phase silica chromatography.

The compounds described herein can modulate activity of one or more of various GPCRs including, for example, A2A/A2B. The term “modulate” is meant to refer to an ability to increase or decrease the activity of one or more members of the A2A/A2B family. Accordingly, the compounds described herein can be used in methods of modulating A2A/A2B by contacting the A2A/A2B with any one or more of the compounds or compositions described herein. In some embodiments, compounds of the present invention can act as inhibitors of one or both of A2A and A2B. In further embodiments, the compounds described herein can be used to modulate activity of A2A/A2B in an individual in need of modulation of the receptor by administering a modulating amount of a compound described herein, or a pharmaceutically acceptable salt thereof. In some embodiments, modulating is inhibiting.

Given that cancer cell growth and survival is impacted by multiple signaling pathways, the present invention is useful for treating disease states characterized by drug resistant mutants. In addition, different GPCR inhibitors, exhibiting different preferences in the GPCRs which they modulate the activities of, may be used in combination. This approach could prove highly efficient in treating disease states by targeting multiple signaling pathways, reduce the likelihood of drug-resistance arising in a cell, and reduce the toxicity of treatments for disease.

GPCRs to which the present compounds bind and/or modulate (e.g., inhibit) include any member of the A2A/A2B family.

In some embodiments, more than one compound described herein is used to inhibit the activity of one GPCR (e.g., A2A)

In some embodiments, more than one compound described herein is used to inhibit more than one GPCR, such as at least two GPCRs (e.g., A2A and A2B).

In some embodiments, one or more of the compounds is used in combination with another GPCR antagonist to inhibit the activity of one GPCR (e.g., A2A or A2B).

The inhibitors of A2A/A2B described herein can be selective. By “selective” is meant that the compound binds to or inhibits a GPCR with greater affinity or potency, respectively, compared to at least one other GPCR. In some embodiments, the compounds described herein are selective inhibitors of A2A or A2B. In some embodiments, the compounds described herein are selective inhibitors of A2A (e.g., over A2B). In some embodiments, the compounds described herein are selective inhibitors of A2B (e.g., over A2A). In some embodiments, selectivity can be at least about 2-fold, 5-fold, 10-fold, at least about 20-fold, at least about 50-fold, at least about 100-fold, at least about 200-fold, at least about 500-fold or at least about 1000-fold. Selectivity can be measured by methods routine in the art. In some embodiments, selectivity can be tested at the biochemical affinity against each GPCR. In some embodiments, the selectivity of compounds described herein can be determined by cellular assays associated with particular A2A/A2B GPCR activity.

As used herein, the term “contacting” refers to the bringing together of indicated moieties in an in vitro system or an in vivo system. For example, “contacting” A2A/A2B with a compound described herein includes the administration of a compound of the present invention to an individual or patient, such as a human, having A2A/A2B, as well as, for example, introducing a compound described herein into a sample containing a cellular or purified preparation containing the A2A/A2B.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) preventing the disease; for example, preventing a disease, condition or disorder in an individual who may be predisposed to the disease, condition or disorder but does not yet experience or display the pathology or symptomatology of the disease; (2) inhibiting the disease; for example, inhibiting a disease, condition or disorder in an individual who is experiencing or displaying the pathology or symptomatology of the disease, condition or disorder (i.e., arresting further development of the pathology and/or symptomatology); and (3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease. In some embodiments, the term “treating” or “treatment” refers to inhibiting or ameliorating the disease.

### Dosing and Administration

In some embodiments, the inhibitor of A2A/A2B, or a pharmaceutically acceptable salt thereof, is administered to the subject in a dosage of from about 0.1 mg to about 1000 mg on a free base basis. In some embodiments, the inhibitor of A2A/A2B, or a pharmaceutically acceptable salt thereof, is administered to the subject in a dosage of from about 1 mg to about 500 mg on a free base basis. In some embodiments, the inhibitor of A2A/A2B, or a pharmaceutically acceptable salt thereof, is administered to the subject in a dosage of from about 5 mg to about 250 mg on a free base basis. In some embodiments, the inhibitor of A2A/A2B, or a pharmaceutically acceptable salt thereof, is administered to the subject in a dosage of from about 10 mg to about 100 mg on a free base basis.

In some embodiments, the inhibitor of A2A/A2B, or a pharmaceutically acceptable salt thereof, is administered to the subject in a dosage selected from about 0.5 mg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, and about 1000 mg on a free base basis. In some embodiments, the inhibitor of A2A/A2B, or a pharmaceutically acceptable salt thereof, is administered to the subject in a dosage ranging from about 0.1 mg to about 500 mg on a free base basis, or any dosage value there between. In some embodiments, the inhibitor of A2A/A2B, or a pharmaceutically acceptable salt thereof, is administered to the subject in a dosage ranging from about 1 mg to about 100 mg on a free base basis, or any dosage value there between.

In some embodiments, the inhibitor of A2A/A2B, or a pharmaceutically acceptable salt thereof, is administered to the subject once-daily, every other day, once-weekly or any time intervals between. In some embodiments, the inhibitor of A2A/A2B, or a pharmaceutically acceptable salt thereof, is administered to the subject once-daily. In some embodiments, the inhibitor of A2A/A2B, or a pharmaceutically acceptable salt thereof, is administered to the subject every other day. In some embodiments, the inhibitor of A2A/A2B, or a pharmaceutically acceptable salt thereof, is administered to the subject once-weekly.

In some embodiments, each of the dosages is administered as a single, once daily dosage. In some embodiments, each of the dosages is administered as a single, once daily oral dosage.

In some embodiments, the inhibitor of PD-1/PD-Li, or a pharmaceutically acceptable salt thereof, is administered to the subject in a dosage of from about 0.1 mg to about 1000 mg on a free base basis. In some embodiments, the inhibitor of PD-1/PD-L1, or a pharmaceutically acceptable salt thereof, is administered to the subject in a dosage of from about 1 mg to about 500 mg on a free base basis. In some embodiments, the inhibitor of PD-1/PD-L1, or a pharmaceutically acceptable salt thereof, is administered to the subject in a dosage of from about 5 mg to about 250 mg on a free base basis. In some embodiments, the inhibitor of PD-1/PD-L1, or a pharmaceutically acceptable salt thereof, is administered to the subject in a dosage of from about 10 mg to about 100 mg on a free base basis.

In some embodiments, the inhibitor of PD-1/PD-Li, or a pharmaceutically acceptable salt thereof, is administered to the subject in a dosage selected from about 0.5 mg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, and about 1000 mg on a free base basis. In some embodiments, the inhibitor of PD-1/PD-L1, or a pharmaceutically acceptable salt thereof, is administered to the subject in a dosage ranging from about 0.1 mg to about 500 mg on a free base basis, or any dosage value there between. In some embodiments, the inhibitor of PD-1/PD-L1, or a pharmaceutically acceptable salt thereof, is administered to the subject in a dosage ranging from about 1 mg to about 100 mg on a free base basis, or any dosage value there between.

In some embodiments, the inhibitor of PD-1/PD-Li is administered to the subject in a dosage of about 1 mg/kg to about 10 mg/kg. In some embodiments, the inhibitor of PD-1/PD-L1 is administered to the subject in a dosage of about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg. In some embodiments, the inhibitor of PD-1/PD-L1 is administered to the subject in a dosage of about 200 mg to about 1000 mg. In some embodiments, the inhibitor of PD-1/PD-L1 is administered to the subject in a dosage of about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg or about 1000 mg.

In some embodiments, the inhibitor of PD-1/PD-Li is administered to the subject once-daily, every other day, once-weekly or any time intervals between. In some embodiments, the inhibitor of PD-1/PD-L1 is administered to the subject once-daily. In some embodiments, the inhibitor of PD-1/PD-L1 is administered to the subject every other day. In some embodiments, the inhibitor of PD-1/PD-L1 is administered to the subject once-weekly.

In some embodiments, each of the dosages is administered as a single, once daily dosage. In some embodiments, each of the dosages is administered as a single, once daily oral dosage.

In some embodiments, the inhibitor of PD-1/PD-L1 is administered to the subject every two weeks, every three weeks or every four weeks. In some embodiments, the inhibitor of PD-1/PD-L1 is administered to the subject monthly or quarterly. In some embodiments, the inhibitor of PD-1/PD-L1 is administered to the subject by intravenous administration.

In some embodiments, the inhibitor of PD-1/PD-L1 is administered to the subject at a dosage of 1 mg/kg Q2W.

In some embodiments, the inhibitor of PD-1/PD-L1 is administered to the subject at a dosage of 3 mg/kg Q2W.

In some embodiments, the inhibitor of PD-1/PD-L1 is administered to the subject at a dosage of 3 mg/kg Q4W.

In some embodiments, the inhibitor of PD-1/PD-L1 is administered to the subject at a dosage of 10 mg/kg Q2W.

In some embodiments, the inhibitor of PD-1/PD-L1 is administered to the subject at a dosage of 10 mg/kg Q4W.

In some embodiments, the inhibitor of PD-1/PD-L1 is administered to the subject at a dosage of 200 mg Q3W.

In some embodiments, the inhibitor of PD-1/PD-L1 is administered to the subject at a dosage of 250 mg Q3W.

In some embodiments, the inhibitor of PD-1/PD-L1 is administered to the subject at a dosage of 375 mg Q3W.

In some embodiments, the inhibitor of PD-1/PD-L1 is administered to the subject at a dosage of 500 mg Q4W.

In some embodiments, the inhibitor of PD-1/PD-L1 is administered to the subject at a dosage of 750 mg Q4W.

In some embodiments, the inhibitor of PD-1/PD-L1 is ANTIBODY X. In some embodiments, the ANTIBODY X is administered to the subject is a dosage of from about 250 mg to about to about 850 mg. In some embodiments, the ANTIBODY X is administered to the subject is a dosage of from about 375 mg to about to about 850 mg.

In some embodiments, the ANTIBODY X is administered to the subject is a dosage of from about 450 mg to about to about 850 mg. In some embodiments, the ANTIBODY X is administered to the subject is a dosage of from about 500 mg to about to about 750 mg.

In some embodiments, the ANTIBODY X is administered to the subject is a dosage of about 500 mg. In some embodiments, the ANTIBODY X is administered to the subject is a dosage of about 750 mg. In some embodiments, the ANTIBODY X is administered to the subject every four weeks. In some embodiments, the ANTIBODY X is administered to the subject by intravenous administration.

In some embodiments, the ANTIBODY X is administered to the subject at a dosage of 1 mg/kg Q2W.

In some embodiments, the ANTIBODY X is administered to the subject at a dosage of 3 mg/kg Q2W.

In some embodiments, the ANTIBODY X is administered to the subject at a dosage of 3 mg/kg Q4W.

In some embodiments, the ANTIBODY X is administered to the subject at a dosage of 10 mg/kg Q2W.

In some embodiments, the ANTIBODY X is administered to the subject at a dosage of 10 mg/kg Q4W.

In some embodiments, the ANTIBODY X is administered to the subject at a dosage of 200 mg Q3W.

In some embodiments, the ANTIBODY X is administered to the subject at a dosage of 250 mg Q3W.

In some embodiments, the ANTIBODY X is administered to the subject at a dosage of 375 mg Q3W.

In some embodiments, the ANTIBODY X is administered to the subject at a dosage of 500 mg Q4W.

In some embodiments, the ANTIBODY X is administered to the subject at a dosage of 750 mg Q4W.

In some embodiments, provided herein is a method of treating a cancer in a subject, comprising administering to the subject:
(i) an inhibitor of A2A/A2B which is 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof; and
(ii) an inhibitor of PD-1/PD-L1 which is ANTIBODY X.

In some embodiments, the inhibitor of A2A/A2B is administered to the subject in a dosage of from about 0.1 mg to about 500 mg on a free base basis, wherein the inhibitor of A2A/A2B is administered once-daily or every other day.

In some embodiments, the ANTIBODY X is administered to the subject in a dosage of about 100 mg to about 1000 mg Q4W.

In some embodiments, provided herein is a method of treating a cancer selected from bladder cancer, breast cancer, cervical cancer, colon cancer, rectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, and Merkel cell carcinoma in a subject, comprising administering to the subject:
(i) an inhibitor of A2A/A2B which is 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof; and
(ii) an inhibitor of PD-1/PD-L1 which is ANTIBODY X;

wherein the inhibitor of A2A/A2B is administered to the subject in a dosage of from about 0.1 mg to about 500 mg on a free base basis, wherein the inhibitor of A2A/A2B is administered once-daily or every other day; and

the ANTIBODY X is administered to the subject in a dosage of about 100 mg to about 1000 mg Q4W.

In some embodiments, the ANTIBODY X is administered to the subject in a dosage of about 375 mg Q4W. In some embodiments, the ANTIBODY X is administered to the subject in a dosage of about 500 mg Q4W. In some embodiments, the ANTIBODY X is administered to the subject in a dosage of about 750 mg Q4W.

In some embodiments, provided herein is a method of treating a cancer in a subject, comprising administering to the subject:
(i) an inhibitor of A2A/A2B which is 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof; and
(ii) an inhibitor of PD-1/PD-L1 which is pembrolizumab.

In some embodiments, provided herein is a method of treating a cancer selected from bladder cancer, breast cancer, cervical cancer, colon cancer, rectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, and Merkel cell carcinoma in a subject, comprising administering to the subject:
(i) an inhibitor of A2A/A2B which is 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof; and
(ii) an inhibitor of PD-1/PD-L1 which is pembrolizumab.

In some embodiments, provided herein is a method of treating a cancer in a subject, comprising administering to the subject:
(i) an inhibitor of A2A/A2B which is 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof; and
(ii) an inhibitor of PD-1/PD-L1 which is atezolizumab.

In some embodiments, provided herein is a method of treating a cancer selected from bladder cancer, breast cancer, cervical cancer, colon cancer, rectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, and Merkel cell carcinoma in a subject, comprising administering to the subject:
(i) an inhibitor of A2A/A2B which is 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof; and
(ii) an inhibitor of PD-1/PD-L1 which is atezolizumab.

In some embodiments, provided herein is a method of treating a cancer in a subject, comprising administering to the subject:
(i) an inhibitor of A2A/A2B which is 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof; and
(ii) an inhibitor of PD-1/PD-L1 which is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof (Compound Y).

In some embodiments, provided herein is a method of treating a cancer selected from bladder cancer, breast cancer, cervical cancer, colon cancer, rectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, and Merkel cell carcinoma in a subject, comprising administering to the subject:
(i) an inhibitor of A2A/A2B which is 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof; and
(ii) an inhibitor of PD-1/PD-L1 which is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof (Compound Y).

In some embodiments, provided herein is a method of treating a cancer in a subject, comprising administering to the subject:
(i) an inhibitor of A2A/A2B which is 3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is ANTIBODY X.

In some embodiments, provided herein is a method of treating a cancer in a subject, comprising administering to the subject:
(i) an inhibitor of A2A/A2B which is 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is ANTIBODY X.

In some embodiments, the inhibitor of A2A/A2B is administered to the subject in a dosage of from about 0.1 mg to about 500 mg on a free base basis, wherein the inhibitor of A2A/A2B is administered once-daily or every other day.

In some embodiments, the ANTIBODY X is administered to the subject in a dosage of about 100 mg to about 1000 mg Q4W.

In some embodiments, provided herein is a method of treating a cancer selected from bladder cancer, breast cancer, cervical cancer, colon cancer, rectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, and Merkel cell carcinoma in a subject, comprising administering to the subject:
(i) an inhibitor of A2A/A2B which is 3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is ANTIBODY X;
wherein the inhibitor of A2A/A2B is administered to the subject in a dosage of from about 0.1 mg to about 500 mg on a free base basis, wherein the inhibitor of A2A/A2B is administered once-daily or every other day; and
the ANTIBODY X is administered to the subject in a dosage of about 100 mg to about 1000 mg Q4W.

In some embodiments, provided herein is a method of treating a cancer selected from bladder cancer, breast cancer, cervical cancer, colon cancer, rectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, and Merkel cell carcinoma in a subject, comprising administering to the subject:
(i) an inhibitor of A2A/A2B which is 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is ANTIBODY X;
wherein the inhibitor of A2A/A2B is administered to the subject in a dosage of from about 0.1 mg to about 500 mg on a free base basis, wherein the inhibitor of A2A/A2B is administered once-daily or every other day; and
the ANTIBODY X is administered to the subject in a dosage of about 100 mg to about 1000 mg Q4W.

In some embodiments, the ANTIBODY X is administered to the subject in a dosage of about 375 mg Q4W. In some embodiments, the ANTIBODY X is administered to the subject in a dosage of about 500 mg Q4W. In some embodiments, the ANTIBODY X is administered to the subject in a dosage of about 750 mg Q4W.

In some embodiments, provided herein is a method of treating a cancer in a subject, comprising administering to the subject:
(i) an inhibitor of A2A/A2B which is 3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is pembrolizumab.

In some embodiments, provided herein is a method of treating a cancer in a subject, comprising administering to the subject:
(i) an inhibitor of A2A/A2B which is 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is pembrolizumab.

In some embodiments, provided herein is a method of treating a cancer selected from bladder cancer, breast cancer, cervical cancer, colon cancer, rectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, and Merkel cell carcinoma in a subject, comprising administering to the subject:
(i) an inhibitor of A2A/A2B which is 3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is pembrolizumab.

In some embodiments, provided herein is a method of treating a cancer selected from bladder cancer, breast cancer, cervical cancer, colon cancer, rectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, and Merkel cell carcinoma in a subject, comprising administering to the subject:
(i) an inhibitor of A2A/A2B which is 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is pembrolizumab.

In some embodiments, provided herein is a method of treating a cancer in a subject, comprising administering to the subject:
(i) an inhibitor of A2A/A2B which is 3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is atezolizumab.

In some embodiments, provided herein is a method of treating a cancer in a subject, comprising administering to the subject:
(i) an inhibitor of A2A/A2B which is 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is atezolizumab.

In some embodiments, provided herein is a method of treating a cancer selected from bladder cancer, breast cancer, cervical cancer, colon cancer, rectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, and Merkel cell carcinoma in a subject, comprising administering to the subject:
(i) an inhibitor of A2A/A2B which is 3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is atezolizumab.

In some embodiments, provided herein is a method of treating a cancer selected from bladder cancer, breast cancer, cervical cancer, colon cancer, rectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, and Merkel cell carcinoma in a subject, comprising administering to the subject:
(i) an inhibitor of A2A/A2B which is 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is atezolizumab.

In some embodiments, provided herein is a method of treating a cancer in a subject, comprising administering to the subject:
(i) an inhibitor of A2A/A2B which is 3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof (Compound Y).

In some embodiments, provided herein is a method of treating a cancer in a subject, comprising administering to the subject:
(i) an inhibitor of A2A/A2B which is 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof (Compound Y).

In some embodiments, provided herein is a method of treating a cancer selected from bladder cancer, breast cancer, cervical cancer, colon cancer, rectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, and Merkel cell carcinoma in a subject, comprising administering to the subject:
(i) an inhibitor of A2A/A2B which is 3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof (Compound Y).

In some embodiments, provided herein is a method of treating a cancer selected from bladder cancer, breast cancer, cervical cancer, colon cancer, rectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, and Merkel cell carcinoma in a subject, comprising administering to the subject:
(i) an inhibitor of A2A/A2B which is 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof (Compound Y).

In some embodiments, the inhibitor of A2A/A2B and the inhibitor of PD-1/PD-L1 are administered simultaneously.

In some embodiments, the inhibitor of A2A/A2B and the inhibitor of PD-1/PD-L1 are administered sequentially.

When the inhibitor of PD-1/PD-L1 is an anti-PD-1 antibody or antigen-binding fragment thereof, it can be administered to a subject, e.g., a subject in need thereof, for example, a human subject, by a variety of methods. The methods and dosages discussed herein are applicable for all anti-PD-1 antibody or antigen-binding fragments thereof, including ANTIBODY X. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the antibody or antigen-binding fragment thereof can also be tailored for the individual case, e.g., by monitoring the subject, e.g., using tomographic imaging, e.g., to visualize a tumor.

The antibody or antigen-binding fragment thereof can be administered as a fixed dose, or in a mg/kg dose. The dose can also be chosen to reduce or avoid production of antibodies against the anti-PD-1 antibody. Dosage regimens are adjusted to provide the desired response, e.g., a therapeutic response or a combinatorial therapeutic effect. Generally, doses of the anti-PD-1 antibody (and optionally a second agent) can be used in order to provide a subject with the agent in bioavailable quantities. For example, doses in the range of 0.1-100 mg/kg, 0.5-100 mg/kg, 1 mg/kg-100 mg/kg, 0.5-20 mg/kg, 0.1-10 mg/kg, or 1-10 mg/kg can be administered. Other doses can also be used. In specific embodiments, a subject in need of treatment with an anti-PD-1 antibody is administered the antibody at a dose of 1 mg/kg, 2 mg/kg, 3 mg/kg, 4 mg/kg, 5 mg/kg, 10 mg/kg, 15 mg/kg, 20 mg/kg, 30 mg/kg, 35 mg/kg, or 40 mg/kg.

A composition may comprise about 1 mg/mL to 100 mg/ml or about 10 mg/mL to 100 mg/ml or about 50 to 250 mg/mL or about 100 to 150 mg/ml or about 100 to 250 mg/ml of anti-PD-1 antibody or antigen-binding fragment thereof.

Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody may be administered via continuous infusion. Exemplary fixed doses include 375 mg, 500 mg and 750 mg.

An anti-PD-1 antibody or antigen-binding fragment thereof dose can be administered, e.g., at a periodic interval over a period of time (a course of treatment) sufficient to encompass at least 2 doses, 3 doses, 5 doses, 10 doses, or more, e.g., once or twice daily, or about one to four times per week, or preferably weekly, biweekly (every two weeks), every three weeks, monthly, e.g., for between about 1 to 12 weeks, preferably between 2 to 8 weeks, more preferably between about 3 to 7 weeks, and even more preferably for about 4, 5, or 6 weeks. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

### Pharmaceutical Formulations

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration.

The compositions of the disclosure can further include one or more additional pharmaceutical agents such as a chemotherapeutic, steroid, anti-inflammatory compound, or immunosuppressant, examples of which are listed herein.

In certain embodiments, the anti-PD-1 antibody may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

### Solid Tumors and Cancers

Examples of cancers that are treatable using the treatment methods and regimens of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The methods of the present disclosure are also useful for the treatment of metastatic cancers, especially metastatic cancers that express PD-Ll.

In some embodiments, cancers treatable with methods of the present disclosure include melanoma (e.g., metastatic malignant melanoma), renal cancer (e.g. clear cell carcinoma), prostate cancer (e.g. hormone refractory prostate adenocarcinoma), breast cancer, colon cancer, lung cancer (e.g. non-small cell lung cancer and small cell lung cancer), squamous cell head and neck cancer, urothelial cancer (e.g. bladder) and cancers with high microsatellite instability (MSIhigh). Additionally, the disclosure includes refractory or recurrent malignancies whose growth may be inhibited using the methods of the disclosure.

In some embodiments, cancers that are treatable using the methods of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma or multiple myeloma) and combinations of said cancers.

In some embodiments, cancers that are treatable using the methods of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, the cancer is selected from lung cancer (e.g., non-small cell lung cancer), melanoma, pancreatic cancer, breast cancer, prostate cancer, liver cancer, colon cancer, endometrial cancer, bladder cancer, skin cancer, cancer of the uterus, ovarian cancer, cancer of the head or neck, thyroid cancer, renal cancer, gastric cancer, and sarcoma. In some embodiments, the cancer is selected from acute lymphoblastic leukemia, acute myelogenous leukemia, chronic lymphocytic leukemia, chronic myelogenous leukemia, diffuse large-B cell lymphoma, mantle cell lymphoma, non-Hodgkin lymphoma, Hodgkin lymphoma, multiple myeloma, polycythemia vera, essential thrombocythemia, chronic myelogenous leukemia, myelofibrosis, primary myelofibrosis, post-polycythemia vera/essential thrombocythemia myelofibrosis, post-essential thrombocythemia myelofibrosis and post-polycythemia vera myelofibrosis. In some embodiments, the cancer is selected from melanoma, endometrial cancer, lung cancer, renal cell carcinoma, urothelial carcinoma, bladder cancer, breast cancer, and pancreatic cancer.

In some embodiments, the cancer is selected from bladder cancer, lung cancer (e.g., non-small cell lung cancer (NSCLC), small cell lung cancer, or lung metastasis), melanoma (e.g., metastatic melanoma), breast cancer, cervical cancer, ovarian cancer, colon cancer, rectal cancer, colorectal cancer, pancreatic cancer, esophageal cancer, prostate cancer, kidney cancer, skin cancer, thyroid cancer, liver cancer, uterine cancer, head and neck cancer, renal cell carcinoma, endometrial cancer, anal cancer, cholangiocarcinoma, oral cancer, non-melanoma skin cancer, and Merkel call carcinoma.

In some embodiments, the prostate cancer is metastatic castrate-resistant prostate carcinoma (mCRPC).

In some embodiments, the colorectal cancer is colorectal carcinoma (CRC).

In some embodiments, the cancer is lung cancer (e.g., non-small cell lung cancer), melanoma, pancreatic cancer, breast cancer, head and neck squamous cell carcinoma, prostate cancer, liver cancer, color cancer, endometrial cancer, bladder cancer, skin cancer, cancer of the uterus, renal cancer, gastric cancer, or sarcoma. In some embodiments, the sarcoma is Askin's tumor, sarcoma botryoides, chondrosarcoma, Ewing's sarcoma, malignant hemangioendothelioma, malignant schwannoma, osteosarcoma, alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, rhabdomyosarcoma, synovial sarcoma, or undifferentiated pleomorphic sarcoma.

In some embodiments, the cancer is mesothelioma or adrenocarcinoma. In some embodiments, the disease or disorder is mesothelioma. In some embodiments, the cancer is adrenocarcinoma.

MDSC (myeloid-derived suppressor cells) are a heterogenous group of immune cells from the myeloid lineage (a family of cells that originate from bone marrow stem cells). MDSCs strongly expand in pathological situations such as chronic infections and cancer, as a result of an altered haematopoiesis. MDSCs are discriminated from other myeloid cell types in which they possess strong immunosuppressive activities rather than immunostimulatory properties. Similar to other myeloid cells, MDSCs interact with other immune cell types including T cells, dendritic cells, macrophages and natural killer cells to regulate their functions. In some embodiments, the compounds, etc. described herein can be used in methods related to cancer tissue (e.g., tumors) with high infiltration of MDSCs, including solid tumors with high basal level of macrophage and/or MDSC infiltration. In some embodiments, the combination therapy described herein can be used in methods related to cancer tissue (e.g., tumors) with tumor or tumor infiltrating lymphocytes (TILs) that express PD-1 or PD-L1.

In some embodiments, the cancer is head and neck squamous cell carcinoma (HNSCC), non-small cell lung cancer (NSCLC), colorectal cancer (e.g., colon cancer), melanoma, ovarian cancer, bladder cancer, renal cell carcinoma, liver cancer, or hepatocellular carcinoma.

In some embodiments, the cancer is selected from bladder cancer, breast cancer, cervical cancer, colon cancer, rectal cancer, anal cancer, endometrial cancer, kidney cancer, oral cancer, head and neck cancer, liver cancer, melanoma, mesothelioma, non-small cell lung cancer, small cell lung cancer, non-melanoma skin cancer, ovarian cancer, pancreatic cancer, prostate cancer, sarcoma, thyroid cancer, and Merkel cell carcinoma.

In some embodiments, the cancer is selected from the cancer is selected from melanoma, endometrial cancer, lung cancer, kidney cancer, bladder cancer, breast cancer, pancreatic cancer, and colon cancer.

In some embodiments, the cancer is selected from endometrial cancer, anal cancer, and cholangiocarcinoma.

In some embodiments, the cancer is a tumor that displays high adenosine levels in the tumor microenvironment. These tumors may be enriched by a gene expression signature, or enriched by high expression levels of CD73 and/or other alkaline phosphatases, including tissue nonspecific alkaline phosphatase (i.e., TNAP and PAP).

In some embodiments, the cancer is colon cancer. In some embodiments, the cancer is melanoma. In some embodiments, the cancer is endometrial cancer. In some embodiments, the endometrial cancer is endometrioid adenocarcinoma. In some embodiments, the cancer is lung cancer. In some embodiments, the lung cancer is selected from non-small cell lung cancer and small cell lung cancer. In some embodiments, the cancer is renal cell carcinoma. In some embodiments, the cancer is urothelial carcinoma. In some embodiments, the cancer is bladder cancer. In some embodiments, the cancer is breast cancer. In some embodiments, the breast cancer is triple-negative breast cancer. In some embodiments, the cancer is pancreatic cancer. In some embodiments, the pancreatic cancer is pancreatic ductal adenocarcinoma. In some embodiments, the cancer is a sarcoma. In some embodiments, the sarcoma is selected from Askin's tumor, sarcoma botryoides, chondrosarcoma, Ewing's sarcoma, malignant hemangioendothelioma, malignant schwannoma, osteosarcoma, alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, rhabdomyosarcoma, synovial sarcoma, and undifferentiated pleomorphic sarcoma.

### Labeled Compounds and Assay Methods

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of an alkyl group of a compound described herein can be optionally substituted with deuterium atoms, such as —CD₃ being substituted for —CH₃).

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

In some embodiments, 1, 2, 3, 4, 5, 6, 7, or 8 hydrogen atoms, attached to carbon atoms of the compounds described herein, are optionally replaced by deuterium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro A2A/A2B labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

### Methods of Producing Antibodies

Antibodies may be produced in bacterial or eukaryotic cells. Some antibodies, e.g., Fab's, can be produced in bacterial cells, e.g., E. coli cells. Antibodies can also be produced in eukaryotic cells such as transformed cell lines (e.g., CHO, 293E, COS). In addition, antibodies (e.g., scFv's) can be expressed in a yeast cell such as Pichia (see, e.g., Powers et al., J Immunol Methods. 251:123-35 (2001)), Hanseula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-PD-1 antibody (e.g., ANTIBODY X) is introduced into dhfr CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

Antibodies, such as ANTIBODY X, can be made, for example, by preparing and expressing synthetic genes that encode the recited amino acid sequences or by mutating human germline genes to provide a gene that encodes the recited amino acid sequences. Moreover, this antibody and other anti-PD-1 antibodies can be obtained, e.g., using one or more of the following methods.

Humanized antibodies can be generated by replacing sequences of the Fv variable region that are not directly involved in antigen binding with equivalent sequences from human Fv variable regions. General methods for generating humanized antibodies are provided by Morrison, S. L., Science, 229:1202-1207 (1985), by Oi et al., BioTechniques, 4:214 (1986), and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al., J. Mol. Biol., 227:776-798 (1992); Cook, G. P. et al., Immunol. Today, 16: 237-242 (1995); Chothia, D. et al., J. Mol. Bio. 227:799-817 (1992); and Tomlinson et al., EMBO J., 14:4628-4638 (1995). The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and U.S. Pat. No. 6,407,213; Tempest et al. (1991) Biotechnology 9:266-271. Still another method is termed “humaneering” and is described, for example, in U.S. 2005-008625.

The antibody can include a human Fc region, e.g., a wild-type Fc region or an Fc region that includes one or more alterations. In one embodiment, the constant region is altered, e.g., mutated, to modify the properties of the antibody (e.g., to increase or decrease one or more of: Fc receptor binding, antibody glycosylation, the number of cysteine residues, effector cell function, or complement function). For example, the human IgG1 constant region can be mutated at one or more residues, e.g., one or more of residues 234 and 237 (based on Kabat numbering). Antibodies may have mutations in the CH2 region of the heavy chain that reduce or alter effector function, e.g., Fc receptor binding and complement activation. For example, antibodies may have mutations such as those described in U.S. Pat. Nos. 5,624,821 and 5,648,260. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005-0037000.

The anti-PD-1 antibodies can be in the form of full length antibodies, or in the form of low molecular weight forms (e.g., biologically active antibody fragments or minibodies) of the anti-PD-1 antibodies, e.g., Fab, Fab′, F(ab′)₂, Fv, Fd, dAb, scFv, and sc(Fv)2. Other anti-PD-1 antibodies encompassed by this disclosure include single domain antibody (sdAb) containing a single variable chain such as, VH or VL, or a biologically active fragment thereof. See, e.g., Moller et al., J. Biol. Chem., 285(49): 38348-38361 (2010); Harmsen et al., Appl. Microbiol. Biotechnol., 77(1):13-22 (2007); U.S. 2005/0079574 and Davies et al. (1996) Protein Eng., 9(6):531-7. Like a whole antibody, a sdAb is able to bind selectively to a specific antigen. With a molecular weight of only 12-15 kDa, sdAbs are much smaller than common antibodies and even smaller than Fab fragments and single-chain variable fragments.

Provided herein are compositions comprising a mixture of an anti-PD-1 antibody or antigen-binding fragment thereof and one or more acidic variants thereof, e.g., wherein the amount of acidic variant(s) is less than about 80%, 70%, 60%, 60%, 50%, 40%, 30%, 30%, 20%, 10%, 5% or 1%. Also provided are compositions comprising an anti-PD-1 antibody or antigen-binding fragment thereof comprising at least one deamidation site, wherein the pH of the composition is from about 5.0 to about 6.5, such that, e.g., at least about 90% of the anti-PD-1 antibodies are not deamidated (i.e., less than about 10% of the antibodies are deamidated). In certain embodiments, less than about 5%, 3%, 2% or 1% of the antibodies are deamidated. The pH may be from 5.0 to 6.0, such as 5.5 or 6.0. In certain embodiments, the pH of the composition is 5.5, 5.6, 5.7, 5.8, 5.9, 6.0, 6.1, 6.2, 6.3, 6.4 or 6.5.

An “acidic variant” is a variant of a polypeptide of interest which is more acidic (e.g. as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e. the neutral amide side chain has been converted to a residue with an overall acidic character.

The term “mixture” as used herein in reference to a composition comprising an anti-PD-1 antibody or antigen-binding fragment thereof, means the presence of both the desired anti-PD-1 antibody or antigen-binding fragment thereof and one or more acidic variants thereof. The acidic variants may comprise predominantly deamidated anti-PD-1 antibody, with minor amounts of other acidic variant(s).

In certain embodiments, the binding affinity (K_{D}), on-rate (K_{D} on) and/or off-rate (K_{D} off) of the antibody that was mutated to eliminate deamidation is similar to that of the wild-type antibody, e.g., having a difference of less than about 5 fold, 2 fold, 1 fold (100%), 50%, 30%, 20%, 10%, 5%, 3%, 2% or 1%.

### Antibody Fragments

Antibody fragments (e.g., Fab, Fab′, F(ab′)2, Facb, and Fv) may be prepared by proteolytic digestion of intact antibodies. For example, antibody fragments can be obtained by treating the whole antibody with an enzyme such as papain, pepsin, or plasmin. Papain digestion of whole antibodies produces F(ab)2 or Fab fragments; pepsin digestion of whole antibodies yields F(ab′)2 or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J. Immunol., 152:2968-2976 (1994); Better, M. and Horwitz, A. H., Methods in Enzymology, 178:476-496 (1989); Plueckthun, A. and Skerra, A., Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R. E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)2 fragments (Carter et al., Bio/Technology, 10:163-167 (1992)). According to another approach, F(ab′)2 fragments can be isolated directly from recombinant host cell culture. Fab and F(ab′) 2 fragment with increased in vivo half-life comprising a salvage receptor binding epitope residues are described in U.S. Pat. No. 5,869,046.

### Minibodies

Minibodies of anti-PD-1 antibodies include diabodies, single chain (scFv), and single-chain (Fv)2 (sc(Fv)2).

A “diabody” is a bivalent minibody constructed by gene fusion (see, e.g., Holliger, P. et al., Proc. Natl. Acad. Sci. U.S.A, 90:6444-6448 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites.

An scFv is a single-chain polypeptide antibody obtained by linking the VH and VL with a linker (see e.g., Huston et al., Proc. Natl. Acad. Sci. U.S.A, 85:5879-5883 (1988); and Plickthun, “The Pharmacology of Monoclonal Antibodies” Vol. 113, Ed Resenburg and Moore, Springer Verlag, New York, pp. 269-315, (1994)). The order of VHs and VLs to be linked is not particularly limited, and they may be arranged in any order. Examples of arrangements include: [VH] linker [VL]; or [VL] linker [VH]. The H chain V region and L chain V region in an scFv may be derived from any anti-PD-1 antibody or antigen-binding fragment thereof described herein.

An sc(Fv)2 is a minibody in which two VHs and two VLs are linked by a linker to form a single chain (Hudson, et al., J. Immunol. Methods, (1999) 231: 177-189 (1999)). An sc(Fv)2 can be prepared, for example, by connecting scFvs with a linker. The sc(Fv)2 of the present invention include antibodies preferably in which two VHs and two VLs are arranged in the order of: VH, VL, VH, and VL ([VH] linker [VL] linker [VH] linker [VL]), beginning from the N terminus of a single-chain polypeptide; however the order of the two VHs and two VLs is not limited to the above arrangement, and they may be arranged in any order.

### Bispecific Antibodies

Bispecific antibodies are antibodies that have binding specificities for at least two different epitopes. Exemplary bispecific antibodies may bind to two different epitopes of the PD-1 protein. Other such antibodies may combine a PD-1 binding site with a binding site for another protein. Bispecific antibodies can be prepared as full length antibodies or low molecular weight forms thereof (e.g., F(ab′)₂ bispecific antibodies, sc(Fv)2 bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Multivalent Antibodies

A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

### Conjugated Antibodies

The antibodies disclosed herein may be conjugated antibodies which are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g. ⁹⁰Y, ¹³¹I) fluorescent substances, luminescent substances, haptens, enzymes, metal chelates, drugs, and toxins (e.g., calcheamicin, Pseudomonas exotoxin A, ricin (e.g. deglycosylated ricin A chain)).

In one embodiment, to improve the cytotoxic actions of anti-PD-1 antibodies and consequently their therapeutic effectiveness, the antibodies are conjugated with highly toxic substances, including radioisotopes and cytotoxic agents. These conjugates can deliver a toxic load selectively to the target site (i.e., cells expressing the antigen recognized by the antibody) while cells that are not recognized by the antibody are spared. In order to minimize toxicity, conjugates are generally engineered based on molecules with a short serum half-life (thus, the use of murine sequences, and IgG3 or IgG4 isotypes).

In certain embodiments, an anti-PD-1 antibody or antigen-binding fragment thereof are modified with a moiety that improves its stabilization and/or retention in circulation, e.g., in blood, serum, or other tissues, e.g., by at least 1.5, 2, 5, 10, or 50 fold. For example, the anti-PD-1 antibody or antigen-binding fragment thereof can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-PD-1 antibody or antigen-binding fragment thereof can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies can be prepared by performing chemical modifications on the antibodies or the lower molecular weight forms thereof described herein. Methods for modifying antibodies are well known in the art (e.g., U.S. Pat. Nos. 5,057,313 and 5,156,840).

### Kits

The present disclosure also includes pharmaceutical kits useful, for example, in the treatment or prevention of A2A/A2B-associated diseases or disorders described herein, which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

Various modifications of the invention, in addition to those described herein, will be apparent to those skilled in the art from the foregoing description. Such modifications are also intended to fall within the scope of the appended claims. Each reference cited in the present disclosure, including all patent, patent applications, and publications, is incorporated herein by reference in its entirety.

### EXAMPLES

### Example 1: In Vitro CHO-PD-L1 Co-Culture Assay

In vitro the CHO-PD-L1 co-culture assay, T cells were treated in the presence of 20 CHO-PD-L1 cells with PD-1 antibody, and using 5′-N-ethylcarboxamide adenosine (NECA), an adenosine-mimicking reagent, to activate adenosine signaling. Under these conditions, Compound 9 could restore the T cell function with an anti-PD1 reagent.

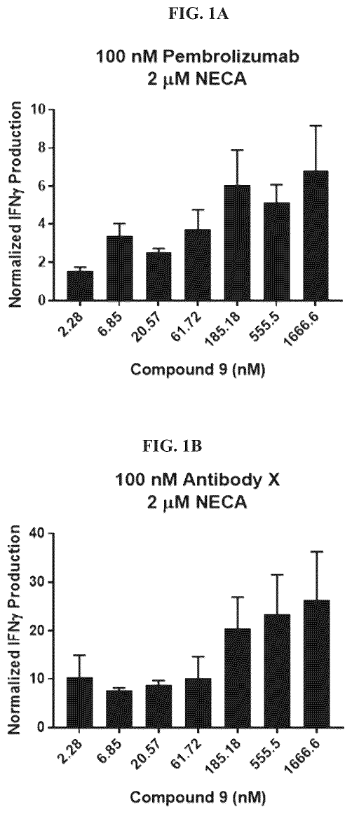

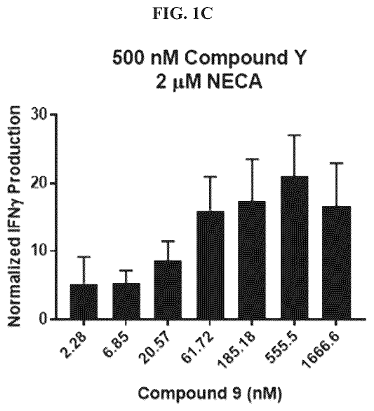

The anti-PD1 reagents tested in this system include: (A) pembrolizumab, (B) Antibody X and (C) Compound Y under the treatment of 2 μM NECA, as shown in FIG. 1.

### Protocol:

Day 0, plate 10,000 CHO PDL1+ cells in Plate 96 Tissue Culture Flat Bottom plate in 100 ul of CHO Media without antibiotics. On day 1, T Cells were thawed and resuspended in T cell media at 1×106 cells/ml. Media was removed from the CHO PDL1+ cells plates and 130 ul of T cell media was added. T cell media at 198 ul was added onto the compound plates at 2 ul, or −1:100 dilution and then re-suspended. 20 ul of compounds from the compound plates were added onto the CHO cell plates at a final dilution of compounds at 1:1000. 50 ul of T cells (50,000 cells) were added onto the plates with CHO cells to make a total of 200 ul volume and incubation was carried out at 37° C. for 72 hrs. After 3 days in culture, the supernatant was collected for an hIFNg and hIL2 cytokine assay run using ProCartaplex 2 plex kits (Life Technologies Cat #PPX-02) for hIFNg and hIL2 (Manufacturer's Protocol). The cytokine assay using ProCartaplex kits are run on a Flex Map 3D Luminex multiplexing platform.

### Example 2: In Vitro Mixed lineage Reaction Assay

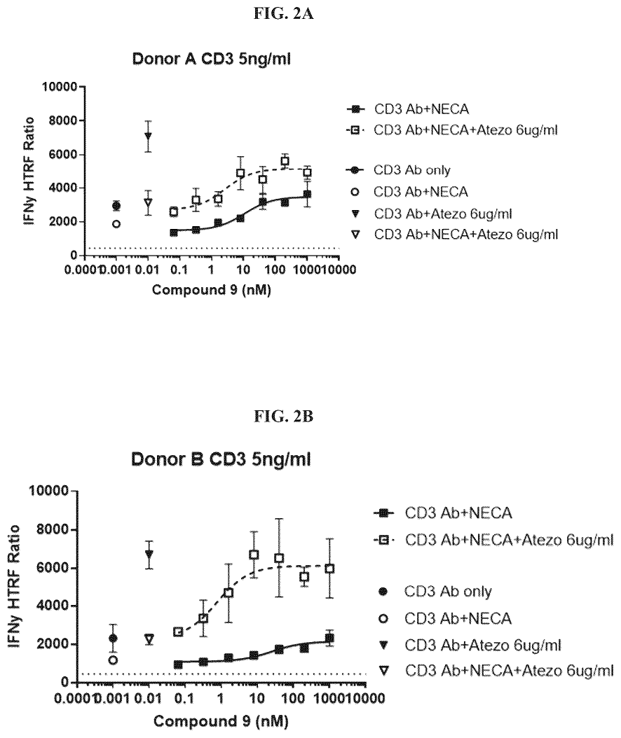

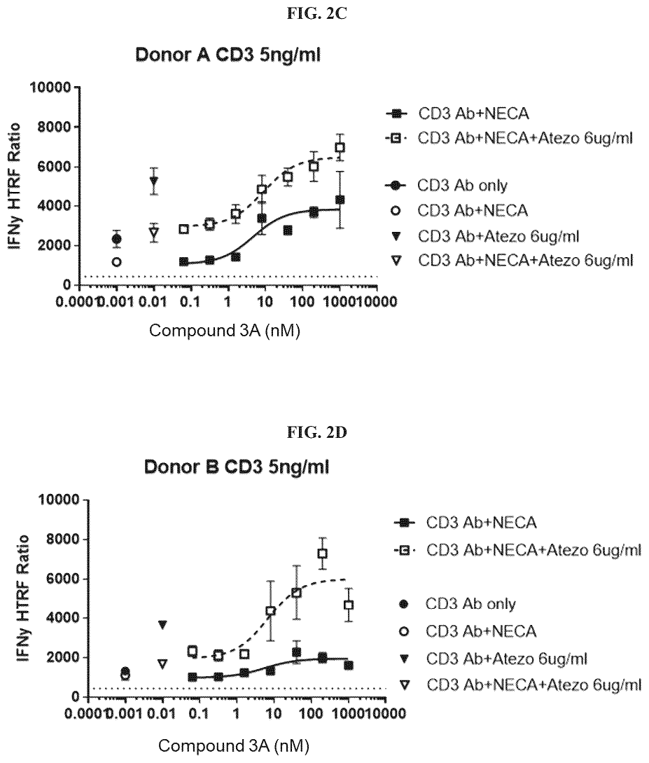

In another in vitro assay, Mixed Lineage Reaction assay (MLR), PBMC from healthy donors were stimulated by CD3 antibody and treated with atezolizumab, Compound 9 or Compound 3A under 1 μM of the adenosine mimicking reagent, NECA 15 (FIGS. 2A-2D).

### Protocol:

On day 0, 10,000 PBMCs from a healthy donor was co-cultured with 10,000 γ-radiated PBMCs from another healthy donor. The cells were plated in a 96-well tissue culture round bottom plate in 200 ul RPMI-1640 media supplemented with 10% FBS, and treated with or without 1 μM NECA, 5 ng/ml CD3 antibody (clone OKT3), and the indicated concentration of compounds/antibody. Cells were incubated at 37° C. for 4 days. IFN-γ in the supernatant of each well was measured by a HTRF kit (Cisbio, 62HIFNGPEH) and the fluorescent signal was detected on a Pherastar FS plate reader (BMG Labtech) on day 4.

Compound 9 when combined with an anti-PD-L1 antibody (i.e., atezolizumab), was able to increase IFNγ production significantly (FIGS. 2A-2B). Compound 3A when combined with an anti-PD-L1 antibody (i.e., atezolizumab), was also able to increase IFNγ production significantly (FIGS. 2C-2D).

### Example 3: In Vivo Efficacy Study in Mouse Synergistic Models

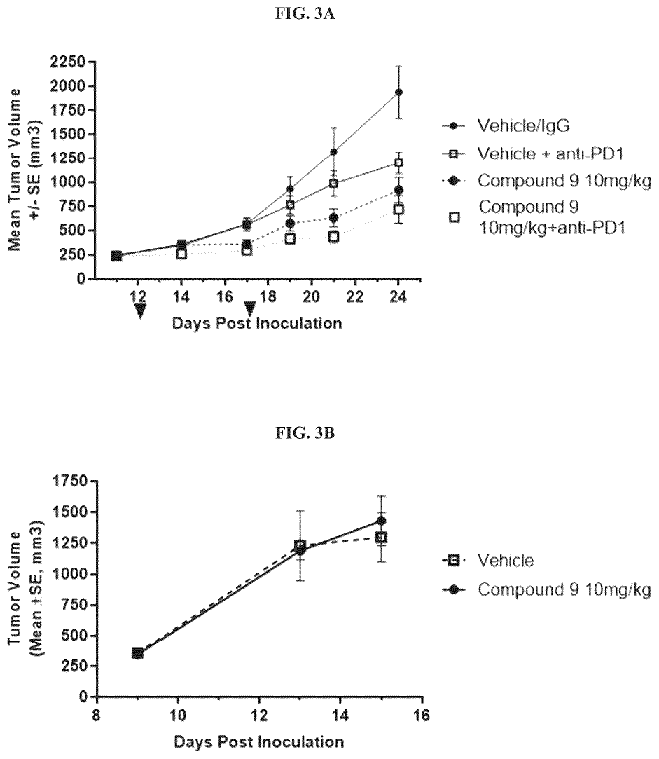

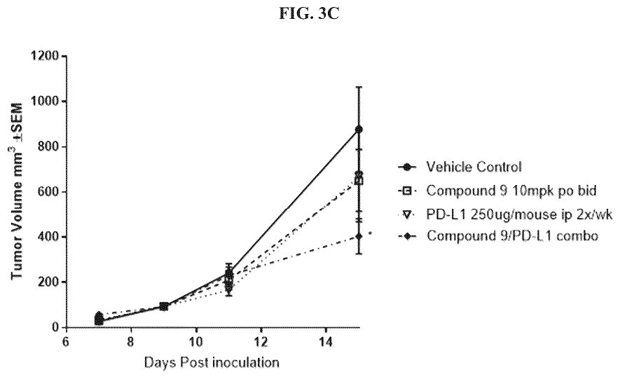

Compound 9 was evaluated for the inhibition of tumor growth in two distinct models. The CT-26 murine colon carcinoma has been demonstrated to have high levels of adenosine in the tumor microenvironment and reflective of high adenosine tumors selected for clinical investigation (Mosely, et al., Cancer Immunol Res; 5(1) January 2017, pp. 29-41). As a single agent, at 10 mg/kg BID, Compound 9 significantly slowed tumor growth at 52% tumor growth inhibition (TGI) relative to the vehicle control, and additionally showed additivity in combination with an anti-PD-1 antibody (77% TGI relative to vehicle) (FIG. 3A). In contrast, no single agent efficacy was observed when the same regimen was applied to the model when hosted in NSG mice, lacking T and NK cells through which Compound 9 is thought to exert most of its therapeutic action (FIG. 3B).

Compound 9 was further evaluated in the B16 melanoma model, an immunologically cold model, for its ability to break immune checkpoint resistance. Both Compound 9 and anti-PD-L1 had modest but insignificant single-agent activity, though when combined, synergized to yield 54% tumor growth inhibition (FIG. 3C). These data suggest that Compound 9 can alter the microenvironment in high adenosine tumors and cooperate with other immune-oncology agents to drive an effective anti-tumor response.

### Example A: Activity of A2A/A2B Inhibitors

### I. A2A Tag-lite® HTRF Assay

Assays were conducted in black low volume 384-well polystyrene plates (Greiner 784076-25) in a final volume of 10 μL. Test compounds were first serially diluted in DMSO and 100 nl added to the plate wells before the addition of other reaction components. The final concentration of DMSO was 1%. Tag-lite® Adenosine A2A labeled cells (CisBio C1TT1A2A) were diluted 1:5 into Tag-lite buffer (CisBio LABMED) and spun 1200 g for 5 mins. The pellet was resuspended at a volume 10.4× the initial cell suspension volume in Tag-lite buffer, and Adenosine A2A Receptor Red antagonist fluorescent ligand (CisBio L0058RED) added at 12.5 nM final concentration. 10 ul of the cell and ligand mix was added to the assay wells and incubated at room temperature for 45 minutes before reading on a PHERAstar FS plate reader (BMG Labtech) with HTRF 337/620/665 optical module. Percent binding of the fluorescent ligand was calculated; where 100 nM of A2A antagonist control ZM 241385 (Tocris 1036) displaces the ligand 100% and 1% DMSO has 0% displacement. The % binding data versus the log of the inhibitor concentration was fitted to a one-site competitive binding model (GraphPad Prism version 7.02) where the ligand constant=12.5 nM and the ligand Kd=1.85 nM. The Kᵢ data obtained via this method are shown in Table 2.

II. Adenosine A2B Receptor cyclic AMP GS Assay

Stably transfected HEK-293 cells expressing the human adenosine A2B receptor (Perkin Elmer) were maintained in MEM culture medium with 10% FBS and 100 μg/ml Geneticin (Life Technologies). 18 to 24 hours prior to assay, geneticin was removed from culture. The cisbio cAMP-GS Dynamic kit utilizing the FRET (Fluorescence Resonance Energy Transfer) technology was used to measure cAMP accumulation in the cells. Compounds of the present disclosure at an appropriate concentration were mixed with 10000 cells/well in white 96 well half area plates (Perkin Elmer) for 30 min at RT gently shaking. Agonist, NECA (R&D Technologies) at 12 nM was added to each well for 60 min at RT gently shaking. Detection reagents, d2-labeled cAMP (acceptor) and anti-cAMP cryptate (donor) were added to each well for 60 min at RT gently shaking. Plates were read on Pherastar (BMG Labtech), fluorescence ratio 665/620 was calculated and EC₅₀ determination was performed by fitting the curve of percent of control versus the log of the compound concentration using GraphPad Prism. The EC₅₀ data obtained via this method are shown in Table 2.

TABLE 2
The A_{2A}_Ki data (Example A(I)) and A_{2B}_cAMP_EC₅0
data (Example A(II)) are provided below.

| Comp. | A_{2A}_Ki | A_{2B}_cAMP_EC₅₀ |
|---|---|---|
| No. | (nM) | (nM) |
| 1 | † | † |
| 2 | † | † |
| 3 | † | † |
| 4 | † | † |
| 5 | † | † |
| 6 | † | † |
| 7 | † | † |
| 8 | † | †† |
| 9 | † | † |
| 10 | † | † |
| 11 | † | † |
| 12 | † | †† |
| 13 | † | † |
| 14 | † | † |
| 15 | † | † |
| 16 | † | † |
| 17 | † | †† |
| 18 | † | † |
| 19 | † | † |
| 20 | † | † |
| 21 | † | † |

† indicates A_{2A}_Kᵢ or A_{2B}_cAMP_EC₅₀ ≤ 10 nM,
†† indicates A_{2A}_Kᵢ or A_{2B}_cAMP_EC₅₀ > 10 nM but ≤ 100 nM,
††† indicates A_{2A}_Kᵢ or A_{2B}_cAMP_EC₅₀ > 100 nM but ≤ 1 μM,
†††† indicates A_{2A}_Kᵢ or A_{2B}_cAMP_EC₅₀ is greater than 1 μM.

### Example A1: Synthesis of 3-(5-Amino-2-(pyridin-2-ylmethyl)-8-(pyrimidin-4-yl)-[1,2,4]triazolo[1,5-c]pyrimidin-7-yl)benzonitrile (Compound 1)

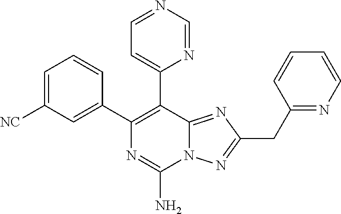 [SMILES: N#Cc1cccc(-c2nc(N)n3nc(Cc4ccccn4)nc3c2-c2ccncn2)c1]

### Step 1: 3-(2-Amino-6-chloropyrimidin-4-yl)benzonitrile

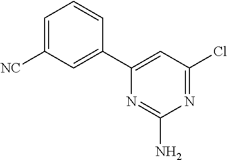 [SMILES: N#Cc1cccc(-c2cc(Cl)nc(N)n2)c1]

A mixture of 4,6-dichloropyrimidin-2-amine (2.5 g, 15.2 mmol), (3-cyanophenyl)boronic acid (2.02 g, 13.7 mmol), tetrakis(triphenylphosphine)palladium(0) (1.06 g, 0.92 mmol) and sodium carbonate (3.23 g, 30.5 mmol) in 1,4-dioxane (60 mL), and water (5 mL) was degassed with nitrogen, then the resulting mixture was heated and stirred at 60° C. for two days. After cooled to room temperature (r.t.), the mixture was concentrated, diluted with water, and extracted with DCM (30 mL×3). The combined organic layers were dried over MgSO₄, filtered, and concentrated. The resulting residue was purified by flash chromatography on a silica gel column eluting with 8% EtOAc in dichloromethane to afford the desired product. LCMS calculated for C₁₁H₈ClN₄ (M+H)⁺: 231.0. Found: 231.0.

### Step 2: 2-(Pyridin-2-yl)acetohydrazide

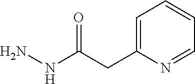 [SMILES: NNC(=O)Cc1ccccn1]

Hydrazine (4.15 mL, 132 mmol) was added to a ethanol (66 mL) solution of methyl 2-(pyridin-2-yl)acetate (10 g, 66.2 mmol) at r.t. The mixture was heated and stirred at 85° C. for 4 h, and then cooled to r.t. White solid was formed upon standing, which was collected via filtration and used in next step without further purification. LCMS calculated for C₇HioN₃0 (M+H)⁺: 152.1. Found: 152.0.

### Step 3: 3-(5-Amino-2-(pyridin-2-ylmethyl)-[1,2,4]triazolo[1,5-c]pyrimidin-7-yl)benzonitrile

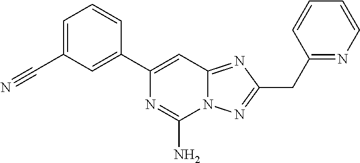 [SMILES: N#Cc1cccc(-c2cc3nc(Cc4ccccn4)nn3c(N)n2)c1]

2-(pyridin-2-yl)acetohydrazide (2.62 g, 17.34 mmol) was added to a ethanol (35 mL) solution of 3-(2-amino-6-chloropyrimidin-4-yl)benzonitrile (4.00 g, 17.34 mmol) at r.t. After being heated and stirred at reflux for 2 h, the reaction mixture was cooled to r.t., and concentrated. The resulting residue was taken into N,O-bis(trimethylsilyl)acetamide (20 mL) and stirred at 120° C. for 7 h. The mixture was then cooled to r.t., poured onto ice, and allowed to stir at r.t. for 1 h. The resulting solid was collected by filtration, and taken into 20 mL of 1 N HCl solution. The resulting mixture was stirred at r.t. for 1 h, filtered, and the aqueous layer was neutralized by addition of saturated NaHCO₃ solution. The resulting precipitate was collected by filtration, and dried to obtain the desired product as a brown solid. LCMS calculated for C₁₈H₁₄N₇(M+H)⁺: 328.1; found 328.1.

### Step 4: 3-(5-Amino-8-bromo-2-(pyridin-2-ylmethyl)-[1,2,4]triazolo[1,5-c]pyrimidin-7-yl)benzonitrile

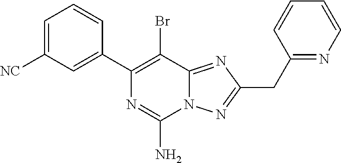 [SMILES: N#Cc1cccc(-c2nc(N)n3nc(Cc4ccccn4)nc3c2Br)c1]

To a mixture of 3-(5-amino-2-(pyridin-2-ylmethyl)-[1,2,4]triazolo[1,5-c]pyrimidin-7-yl)benzonitrile (2 g, 6.11 mmol) in DMF (12 mL) at −30° C. was added NBS (1.09 g, 6.11 mmol) portion-wise. The reaction mixture was allowed to slowly warm to 0° C., resulting a homogenous solution. After stirring at 0° C. for 1 h, the reaction mixture was diluted with saturated NaHCO₃ solution and the resulting solid was collected by filtration. The solid was then purified by flash chromatography on a silica gel column eluting with 0 to 10% MeOH in DCM to afford the desired product. LCMS calculated for C₁₈H₁₃BrN₇ (M+H)⁺: 406.0; found 406.0.

### Step 5: 3-(5-Amino-2-(pyridin-2-ylmethyl)-8-(pyrimidin-4-yl)-[1,2,4]triazolo[1,5-c]pyrimidin-7-yl)benzonitrile

Pd(Ph₃P)₄ (284 mg, 0.246 mmol) was added to a mixture of 4-(tributylstannyl)pyrimidine (1090 mg, 2.95 mmol), 3-(5-amino-8-bromo-2-(pyridin-2-ylmethyl)-[1,2,4]triazolo[1,5-c]pyrimidin-7-yl)benzonitrile (1000 mg, 2.46 mmol), and copper(I) chloride (244 mg, 2.46 mmol) in 1,4-dioxane (12 mL). The reaction mixture was purged with N₂ and stirred at 80° C. for 7 h. The resulting mixture was cooled to r.t., concentrated, diluted with DCM (50 mL) and washed with saturated NH₄OH solution. The organic layer was dried over Na₂SO₄, concentrated, and purified by preparative LC-MS (pH 2, acetonitrile/water with TFA) to afford the product as a TFA salt. LCMS calculated for C₂₂H₁₆N₉(M+H)⁺: 406.2; found 406.2. ¹H NMR (500 MHz, DMSO) δ 8.95 (s, 1H), 8.83 (d, J=5.3 Hz, 1H), 8.59 (d, J=5.1 Hz, 1H), 7.96 (m, 1H), 7.88 (d, J=5.1 Hz, 1H), 7.82 (d, J=7.6 Hz, 1H), 7.76 (s, 1H), 7.60-7.53 (m, 2H), 7.53-7.48 (m, 1H), 7.48-7.42 (m, 1H), 4.49 (s, 2H).

### Example A2: Synthesis of 3-(5-Amino-2-((2,6-difluorophenyl)(hydroxy)methyl)-8-(pyrimidin-4-yl)-[1,2,4]triazolo[1,5-c]pyrimidin-7-yl)benzonitrile (Compound 2)

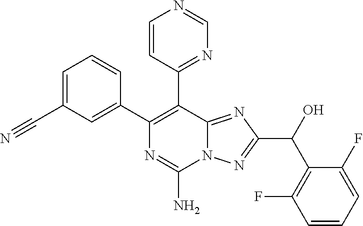 [SMILES: N#Cc1cccc(-c2nc(N)n3nc(C(O)c4c(F)cccc4F)nc3c2-c2ccncn2)c1]

### Step 1: Methyl 2-(2,6-difluorophenyl)-2-hydroxyacetate

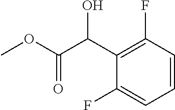

Concentrated sulfuric acid (1.42 mL, 27 mmol) was added to a methanol (45 mL) solution of 2,6-difluoromandelic acid (5 g, 27 mmol) at 0° C. The mixture was stirred at r.t. for 4 h before being concentrated. To the resulting slurry was added saturated NaHCO₃ solution (30 mL). The resulting mixture was extracted with DCM (3×20 mL). The combined organic layers were washed with water, dried over Mg₂SO₄, filtered, and concentrated to afford the crude product, which was used in the next step without further purification. LC-MS calculated for C₁₁H₁₂F₂NO₃ (M+H+MeCN)⁺: m/z=244.1; found 244.2.

### Step 2: 3-(5-Amino-2-((2,6-difluorophenyl)(hydroxy)methyl)-8-(pyrimidin-4-yl)-[1,2,4]triazolo[1,5-c]pyrimidin-7-yl)benzonitrile

This compound was prepared using similar procedures as described for Example A1, with methyl 2-(2,6-difluorophenyl)-2-hydroxyacetate replacing methyl 2-(pyridin-2-yl)acetate in Step 2. The two enantiomers were separated by chiral SFC using a Phenomenex Lux Cellulose-1 column (21.2×250 mm, 5 m particle size) eluting with an isocratic mobile phase 25% MeOH in CO₂ with a flow rate of 80 mL/minute. Peak 1 was isolated, and further purified by prep-LCMS (pH=2, MeCN/water with TFA) to give the desired product as a TFA salt. LC-MS calculated for C₂₃H₁₅F₂N₈O (M+H)⁺: m/z=457.1; found 457.1. ¹H NMR (500 MHz, DMSO) δ 8.94 (d, J=1.3 Hz, 1H), 8.81 (d, J=5.2 Hz, 1H), 7.85 (dd, J=5.3, 1.4 Hz, 1H), 7.81 (dt, J=7.4, 1.5 Hz, 1H), 7.76 (t, J=1.7 Hz, 1H), 7.55 (dt, J=7.8, 1.5 Hz, 1H), 7.49 (t, J=7.8 Hz, 1H), 7.44 (tt, J=8.4, 6.4 Hz, 1H), 7.09 (t, J=8.3 Hz, 2H), 6.27 (s, 1H).

### Example A3: Synthesis of 3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile (Compound 3A) and 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile (Compound 3B)

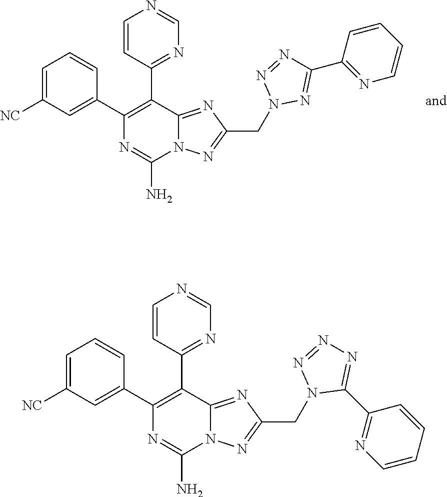 [SMILES: N#Cc1cccc(-c2nc(N)n3nc(Cn4nnc(-c5ccccn5)n4)nc3c2-c2ccncn2)c1 ; N#Cc1cccc(-c2nc(N)n3nc(Cn4nnnc4-c4ccccn4)nc3c2-c2ccncn2)c1]

### Step 1: 3-(5-Amino-2-(hydroxymethyl)-[1,2,4]triazolo[1,5-c]pyrimidin-7-yl)benzonitrile

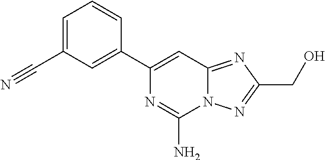 [SMILES: N#Cc1cccc(-c2cc3nc(CO)nn3c(N)n2)c1]

2-Hydroxyacetohydrazide (2.34 g, 26.01 mmol) was added to a ethanol (35 mL) solution of 3-(2-amino-6-chloropyrimidin-4-yl)benzonitrile (4.00 g, 17.34 mmol) (Example A1, Step 1) at r.t. After being heated and stirred at reflux for 2 h, the reaction mixture was cooled to r.t., and concentrated. The resulting residue was taken into N,O-bis(trimethylsilyl)acetamide (20 mL) and stirred at 120° C. for 7 h. The mixture was then cooled to r.t., poured onto ice, and allowed to stir at r.t. for 1 h. The resulting solid was collected by filtration, and taken into 20 mL of 1 N HCl solution. The resulting mixture was stirred at r.t. for 1 h, filtered, and the aqueous layer was neutralized by addition of saturated NaHCO₃ solution. The resulting precipitate was collected by filtration, and dried to obtain the desired product as a brown solid. LCMS calculated for C₁₃H₁₁N₆O (M+H)⁺: 267.1; found 267.1.

### Step 2: 3-(5-Amino-8-bromo-2-(hydroxymethyl)-[1,2,4]triazolo[1,5-c]pyrimidin-7-yl)benzonitrile

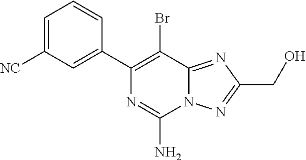 [SMILES: N#Cc1cccc(-c2nc(N)n3nc(CO)nc3c2Br)c1]

To a mixture of 3-(5-amino-2-(hydroxymethyl)-[1,2,4]triazolo[1,5-c]pyrimidin-7-yl)benzonitrile (1.0 g, 3.76 mmol) in DMF (12 mL) at −30° C. was added NBS (0.67 g, 3.76 mmol) portion-wise. The reaction mixture was allowed to slowly warm to 0° C., resulting a homogenous solution. After stirring at 0° C. for 1 h, the reaction mixture was diluted with saturated NaHCO₃ solution and the desired product was collected by filtration and dried. LCMS calculated for C₁₃H₁₀BrN₆O (M+H)⁺: 345.0; found 345.0.

### Step 3: 3-(5-Amino-2-(hydroxymethyl)-8-(pyrimidin-4-yl)-[1,2,4]triazolo[1,5-c]pyrimidin-7-yl)benzonitrile

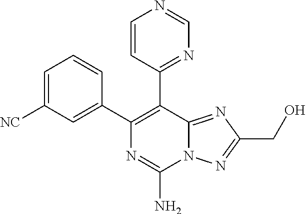 [SMILES: N#Cc1cccc(-c2nc(N)n3nc(CO)nc3c2-c2ccncn2)c1]

Tetrakis(triphenylphosphine)palladium(0) (0.067 g, 0.058 mmol) was added to a mixture of 4-(tributylstannyl)pyrimidine (0.321 g, 0.869 mmol), 3-(5-amino-8-bromo-2-(hydroxymethyl)-[1,2,4]triazolo[1,5-c]pyrimidin-7-yl)benzonitrile (0.20 g, 0.579 mmol), CsF (0.176 g, 1.159 mmol), and copper(I) iodide (0.022 g, 0.116 mmol) in 1,4-dioxane (5.0 mL). The reaction mixture was purged with N₂ and stirred at 80° C. for 7 h. The resulting mixture was cooled to r.t., concentrated and purified by flash column chromatopraphy eluting with 0% to 10% methanol in DCM to afford the product. LC-MS calculated for C₁₇H₁₃N₈O (M+H)⁺: 345.1; found 345.1.

### Step 4: 3-(5-Amino-2-(chloromethyl)-8-(pyrimidin-4-yl)-[1,2,4]triazolo[1,5-c]pyrimidin-7-yl)benzonitrile

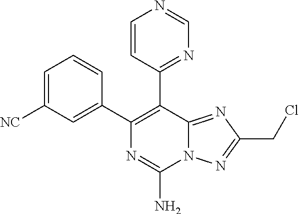 [SMILES: N#Cc1cccc(-c2nc(N)n3nc(CCl)nc3c2-c2ccncn2)c1]

To a mixture of 3-(5-amino-2-(hydroxymethyl)-8-(pyrimidin-4-yl)-[1,2,4]triazolo[1,5-c]pyrimidin-7-yl)benzonitrile (0.1 g, 0.290 mmol) in Acetonitrile (10 ml) was added thionyl chloride (0.212 ml, 2.90 mmol) at r.t. The reaction mixture was stirred at r.t. for 5 h, concentrated, and purified by flash chromatography eluting with 0% to 5% methanol in DCM to afford the product. LC-MS calculated for C₁₇H₁₂ClN₈ (M+H)⁺: 363.1; found 363.1.

### Step 5: Mixture of 3-(5-amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile (Compound 3A) and 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile (Compound 3B)

A mixture of 3-(5-amino-2-(chloromethyl)-8-(pyrimidin-4-yl)-[1,2,4]triazolo[1,5-c]pyrimidin-7-yl)benzonitrile (10 mg, 0.028 mmol), 2-(1H-tetrazol-5-yl)pyridine (8.1 mg, 0.055 mmol) and Cs₂CO₃ (20.7 mg, 0.064 mmol) in DMF (1 mL) was stirred at 100° C. for 10 min. The reaction mixture was then cooled to r.t., diluted with methanol (4 mL), and purified by preparative LC-MS (pH 2, acetonitrile/water with TFA) to afford the product as a TFA salt. LCMS calculated for C₂₃H₁₆N₁₃ (M+H)⁺: 474.2; found 474.2. Compound 3A: ¹H NMR (500 MHz, DMSO) δ 8.99 (d, J=1.4 Hz, 1H), 8.85 (d, J=5.3 Hz, 1H), 8.80-8.71 (m, 1H), 8.71-8.39 (b, 2H), 8.18 (d, J=7.7, 1.1 Hz, 1H), 8.04 (t, J=7.8, 1.8 Hz, 1H), 7.85 (m, 2H), 7.80-7.76 (m, 1H), 7.62-7.55 (m, 2H), 7.53 (t, J=7.8 Hz, 1H), 6.39 (s, 2H).

### Example A4: Synthesis of 3-(5-Amino-2-((3-methylpyridin-2-yl)methoxy)-8-(pyrimidin-4-yl)-[1,2,4]triazolo[1,5-c]pyrimidin-7-yl)benzonitrile (Compound 4)

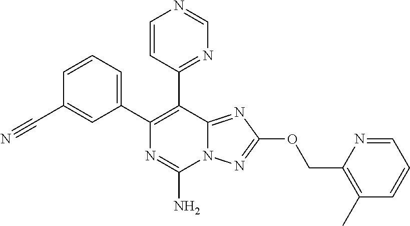 [SMILES: Cc1cccnc1COc1nc2c(-c3ccncn3)c(-c3cccc(C#N)c3)nc(N)n2n1]

### Step 1: 6-Chloro-N²,N²-bis(4-methoxybenzyl)pyrimidine-2,4-diamine

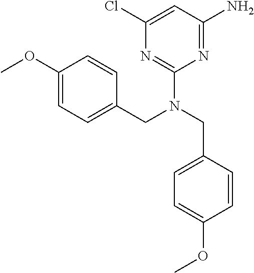 [SMILES: COc1ccc(CN(Cc2ccc(OC)cc2)c2nc(N)cc(Cl)n2)cc1]

To a solution of 2,6-dichloropyrimidin-4-amine (5.0 g, 31 mmol) in 2-propanol (31 mL) was added N,N-diisopropylethylamine (6.4 ml, 37 mmol) and bis(4-methoxybenzyl)amine (7.9 g, 31 mmol). The resulting solution was stirred at 100° C. for 16 h, cooled to r.t., diluted with water (100 mL), and extracted with EtOAc (100 mL). The organic layer was washed with water and brine, dried over anhydrous sodium sulfate, and concentrated to yield the crude product, which was used in the next step without further purification. LC-MS calculated for C₂₀H₂₂ClN₄O₂(M+H)⁺: 385.1; found 385.1.

### Step 2: 7-Chloro-N⁵,N⁵-bis(4-methoxybenzyl)-[1,2,4]triazolo[1,5-c]pyrimidine-2,5-diamine

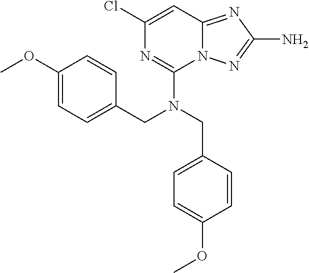 [SMILES: COc1ccc(CN(Cc2ccc(OC)cc2)c2nc(Cl)cc3nc(N)nn23)cc1]

O-ethyl carbonisothiocyanatidate (3.1 mL, 26 mmol) was added to a 1,4-dioxane (5.0 mL) solution of 6-chloro-N²,N²-bis(4-methoxybenzyl)pyrimidine-2,4-diamine (1.0 g, 2.6 mmol) at r.t. The reaction mixture was then stirred at 90° C. overnight, cooled to r.t., and concentrated. The resulting material was dissolved in methanol (12 mL) and ethanol (12 mL), and N,N-diisopropylethylamine (0.91 mL, 5.2 mmol) was added, followed by hydroxylamine hydrochoride (0.54 g, 7.8 mmol). The reaction mixture was stirred at 45° C. for 2 h, cooled to r.t., and concentrated. The resulting material was taken into EtOAc, washed with water, dried over anhydrous sodium sulfate, and concentrated. The crude material was then purified by silica gel chromatography eluting with 0% to 50% EtOAc in hexanes to afford the product. LC-MS calculated for C₂₁H₂₂ClN₆O₂(M+H)⁺: 425.1; found 425.2.

### Step 3: 3-(2-Amino-5-(bis(4-methoxybenzyl)amino)-[1,2,4]triazolo[1,5-c]pyrimidin-7-yl)benzonitrile

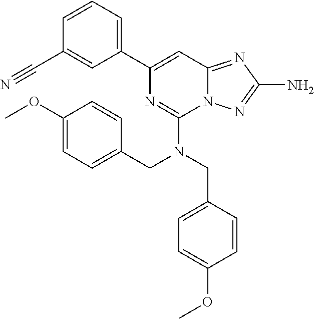 [SMILES: COc1ccc(CN(Cc2ccc(OC)cc2)c2nc(-c3cccc(C#N)c3)cc3nc(N)nn23)cc1]

Chloro(2-dicyclohexylphosphino-2′,4′,6′-tri-i-propyl-1,1′-biphenyl)(2′-amino-1,1′-biphenyl-2-yl) palladium(II) (330 mg, 0.42 mmol) was added to a mixture of (3-cyanophenyl)boronic acid (460 mg, 3.2 mmol), 7-chloro-N⁵,N⁵-bis(4-methoxybenzyl)-[1,2,4]triazolo[1,5-c]pyrimidine-2,5-diamine (890 mg, 2.1 mmol), and sodium carbonate (890 mg, 8.4 mmol) in 1,4-dioxane (8.8 mL) and water (1.8 mL). The mixture was purged with N₂ and stirred at 95° C. overnight. The reaction mixture was then cooled to r.t., concentrated, and purified by silica gel chromatography eluting with 0% to 50% EtOAc in DCM to afford the desired product. LC-MS calculated for C₂₈H₂₆N₇O₂ (M+H)⁺: 492.2; found 492.2.

### Step 4: 3-(2-Amino-5-(bis(4-methoxybenzyl)amino)-8-bromo-[1,2,4]triazolo[1,5-c]pyrimidin-7-yl)benzonitrile

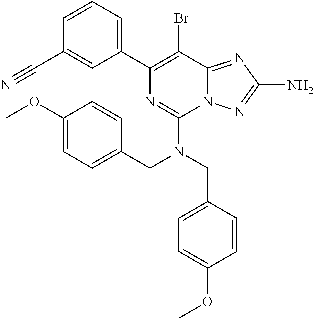 [SMILES: COc1ccc(CN(Cc2ccc(OC)cc2)c2nc(-c3cccc(C#N)c3)c(Br)c3nc(N)nn23)cc1]

To a solution of 3-(2-amino-5-(bis(4-methoxybenzyl)amino)-[1,2,4]triazolo[1,5-c]pyrimidin-7-yl)benzonitrile (330 mg, 0.66 mmol) in DMF (1.4 mL) was slowly added NBS (120 mg, 0.66 mmol) at 0° C. The reaction mixture was then stirred at r.t. for 30 min before water (10 mL) was added. The resulting solid was collected by filtration, and dried to obtain the desired product. LC-MS calculated for C₂₈H₂₅BrN₇O₂(M+H)⁺: m/z=570.1; found 570.2.

### Step 5: 3-(2-Amino-5-(bis(4-methoxybenzyl)amino)-8-(pyrimidin-4-yl)-[1,2,4]triazolo[1,5-c]pyrimidin-7-yl)benzonitrile

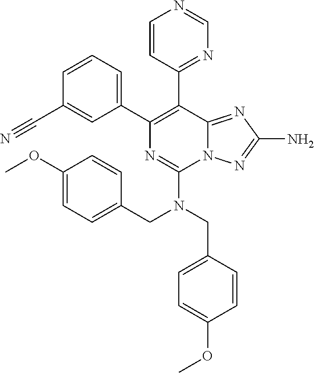 [SMILES: COc1ccc(CN(Cc2ccc(OC)cc2)c2nc(-c3cccc(C#N)c3)c(-c3ccncn3)c3nc(N)nn23)cc1]

A mixture of 3-(2-amino-5-(bis(4-methoxybenzyl)amino)-8-bromo-[1,2,4]triazolo[1,5-c]pyrimidin-7-yl)benzonitrile (350 mg, 0.61 mmol), 4-(tributylstannyl)pyrimidine (210 μL, 0.67 mmol), tetrakis(triphenylphosphine)palladium(0) (70 mg, 0.060 mmol), copper(I) iodide (23 mg, 0.12 mmol) and cesium fluoride (180 mg, 1.2 mmol) in dioxane (4.7 mL) was heated and stirred at 140° C. for 30 min in a microwave reactor. The reaction mixture was then cooled to r.t., filtered through a Celite plug (washed with DCM), and concentrated. The resulting material was purified by silica gel column chromatography eluting with 0-20% MeOH/DCM to give the desired product. LC-MS calculated for C₃₂H₂₈N₉O₂ (M+H)⁺: m/z=570.2; found 570.3.

### Step 6: 3-(5-(Bis(4-methoxybenzyl)amino)-2-bromo-8-(pyrimidin-4-yl)-[1,2,4]triazolo[1,5-c]pyrimidin-7-yl)benzonitrile

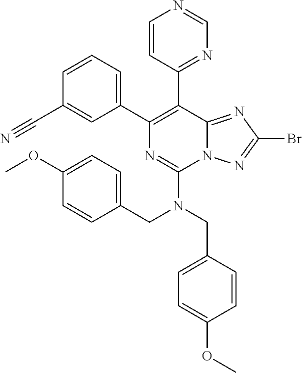 [SMILES: COc1ccc(CN(Cc2ccc(OC)cc2)c2nc(-c3cccc(C#N)c3)c(-c3ccncn3)c3nc(Br)nn23)cc1]

To a mixture of copper(II) bromide (91 mg, 0.407 mmol) and tert-butyl nitrite (0.054 ml, 0.407 mmol) in acetonitrile (3 mL) under nitrogen at 50° C. was added dropwise 3-(2-amino-5-(bis(4-methoxybenzyl)amino)-8-(pyrimidin-4-yl)-[1,2,4]triazolo[1,5-c]pyrimidin-7-yl)benzonitrile (100 mg, 0.203 mmol) in acetonitrile (3 mL). The mixture was stirred at 50° C. for 2 hours. After cooling to room temperature, 1 N aqueous NH₄OH solution (20 mL) was added and the mixture was extracted three times with CH₂Cl₂ (20 mL). The combined organic layers were dried over sodium sulfate, filtered and concentrated. The crude material was purified by silica gel column chromatography eluting with 50-100% ethyl acetate/hexane to give the desired product. LC-MS calculated for C₃₂H₂₆BrN₈O₂(M+H)⁺: m/z=633.1; found 633.2.

### Step 7: 3-(5-Amino-2-((3-methylpyridin-2-yl)methoxy)-8-(pyrimidin-4-yl)-[1,2,4]triazolo[1,5-c]pyrimidin-7-yl)benzonitrile

A suspension of sodium hydride (60% in mineral oil, 3.8 mg, 0.095 mmol), 3-(5-(bis(4-methoxybenzyl)amino)-2-bromo-8-(pyrimidin-4-yl)-[1,2,4]triazolo[1,5-c]pyrimidin-7-yl)benzonitrile (20 mg, 0.032 mmol) and (3-methylpyridin-2-yl)methanol (9.1 μL, 0.095 mmol) in 1,4-dioxane (1 mL) was heated and stirred at 110° C. under nitrogen overnight. The reaction mixture was then cooled to rt, concentrated, and added TFA (1.0 mL). The resulting mixture was then stirred at 110° C. for 30 min, cooled to rt, diluted with acetonitrile, filtered and purified by preparative LC-MS (pH 2, acetonitrile/water with TFA) to give desired product as a TFA salt. LC-MS calculated for C₂₃H₁₈N₉O (M+H)⁺: m/z=436.2; found 436.2. ¹H NMR (600 MHz, DMSO) δ 8.97 (d, J=1.4 Hz, 1H), 8.88 (d, J=5.2 Hz, 1H), 8.58-8.52 (m, 1H), 7.97 (d, J=7.8 Hz, 1H), 7.88 (dd, J=5.4, 1.4 Hz, 1H), 7.85 (dt, J=7.5, 1.5 Hz, 1H), 7.78 (t, J=1.8 Hz, 1H), 7.60-7.54 (m, 2H), 7.53 (t, J=7.8 Hz, 1H), 5.69 (s, 2H), 2.48 (s, 3H).

### Example A5: Synthesis of 3-(5-Amino-2-(hydroxy(phenyl)methyl)-[1,2,4]triazolo[1,5-c]pyrimidin-7-yl)benzonitrile (Compound 5)

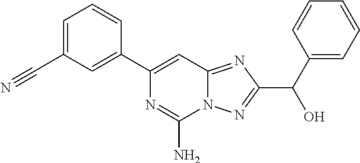 [SMILES: N#Cc1cccc(-c2cc3nc(C(O)c4ccccc4)nn3c(N)n2)c1]

### Step 1: 3-(2-Amino-6-chloropyrimidin-4-yl)benzonitrile

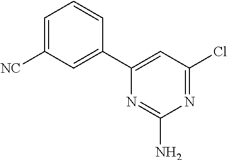 [SMILES: N#Cc1cccc(-c2cc(Cl)nc(N)n2)c1]

A mixture of 4,6-dichloropyrimidin-2-amine (2.5 g, 15.24 mmol), (3-cyanophenyl)boronic acid (2.016 g, 13.72 mmol), tetrakis(triphenylphosphine)palladium(0) (1.057 g, 0.915 mmol) and sodium carbonate (3.23 g, 30.5 mmol) in 1,4-dioxane (60 mL), and water (5 mL) was degassed with nitrogen, then the resulting mixture was heated at 60° C. for two days. After cooled to room temperature (RT), the mixture was concentrated, then diluted with water, and extracted with dichloromethane (DCM, 3×30 mL). The combined organic layers were dried over MgSO₄, filtered, and concentrated. The residue was purified by flash chromatography on a silica gel column with 8% ethyl acetate (EtOAc) in dichloromethane to afford the desired product. LCMS calculated for C₁₁H₈ClN₄ (M+H)⁺: 231.0. Found: 231.0.

### Step 2: 3-(5-Amino-2-(hydroxy(phenyl)methyl)-[1,2,4]triazolo[1,5-c]pyrimidin-7-yl)benzonitrile

A solution of 3-(2-amino-6-chloropyrimidin-4-yl)benzonitrile (100 mg, 0.434 mmol) and 2-hydroxy-2-phenylacetohydrazide (108 mg, 0.650 mmol) in ethanol (2 ml) was heated and stirred at 95° C. for 3 h. After cooling to RT, the reaction mixture was concentrated to dryness, taken into N,O-bis(trimethylsilyl)acetamide (1 mL) and stirred at 120° C. for 7 h. The resulting mixture was cooled to RT, poured onto ice, and stirred for 1 h. The resulting suspension was extracted with DCM three times. The combined organic layers were dried over MgSO₄, filtered, and concentrated. The residue was dissolved in methanol (MeOH) and purified by preparative LC-MS (pH 2, acetonitrile/water with TFA) to afford the product as TFA salt. LCMS calculated for C₁₉H₁₅N₆O (M+H)⁺: 343.1; found 343.1.

### Example A6: Synthesis of 3-(5-Amino-2-((2,6-difluorophenyl)(hydroxy)methyl)-[1,2,4]triazolo[1,5-c]pyrimidin-7-yl)-2-fluorobenzonitrile (Compound 6)

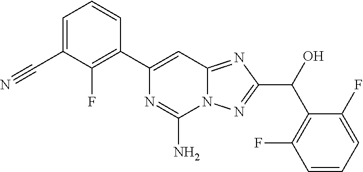 [SMILES: N#Cc1cccc(-c2cc3nc(C(O)c4c(F)cccc4F)nn3c(N)n2)c1F]

### Step 1: 3-(2-Amino-6-chloropyrimidin-4-yl)-2-fluorobenzonitrile

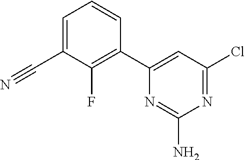 [SMILES: N#Cc1cccc(-c2cc(Cl)nc(N)n2)c1F]

To a solution of 3-bromo-2-fluorobenzonitrile (18.3 g, 91 mmol) in THF (60 mL) cooled to 0° C. was added i-PrMgCl LiCl complex (70.4 mL, 91 mmol) in THF (1.3 M) over 20 min. The mixture was stirred at 0° C. for 50 min, then zinc chloride (48.1 mL, 91 mmol) in 2-MeTHF (1.9 M) was added at 0° C. The reaction was stirred at r.t. for 25 min, at which point 4,6-dichloropyrimidin-2-amine (10 g, 61.0 mmol) was added in one portion. The solution was stirred for 10 min. Tetrakis(triphenylphosphine)palladium (1.41 g, 1.22 mmol) was added to the mixture and the reaction was stirred at r.t. for 16 h. Upon completion, 2,4,6-trimercaptotriazine silica gel (2 g) was added to the reaction solution. The mixture was stirred for 1 h and filtered. The solid was washed with ethyl acetate until the desired product had eluted completely (as detected by LCMS). The filtrate was washed with saturated ammonium chloride solution and water. The organics were concentrated to afford the crude product. Water was added to the crude material and the resulting precipitate was collected by filtration and dried under a stream of nitrogen. The crude material was taken forward without additional purification. LC-MS calculated for C₁₁H₇ClFN₄ (M+H)⁺: m/z=249.0; found 249.0.

### Step 2: Methyl 2-(2,6-difluorophenyl)-2-hydroxyacetate

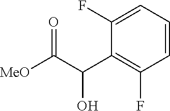 [SMILES: COC(=O)C(O)c1c(F)cccc1F]

Concentrated sulfuric acid (1.4 mL, 27 mmol) was added to a methanol (45 mL) solution of 2,6-difluoromandelic acid (5.0 g, 27 mmol) at 0° C. The mixture was stirred at r.t. for 4 h before being concentrated. To the resulting slurry was added saturated NaHCO₃ solution. The resulting mixture was extracted with DCM. The combined organic layers were washed with water, dried over MgSO₄, filtered, and concentrated to afford the crude product, which was used in the next step without further purification. LC-MS calculated for C₁₁H₁₂F₂NO₃ (M+H+MeCN)⁺: m/z=244.1; found 244.2.

### Step 3: 2-(2,6-Difluorophenyl)-2-hydroxyacetohydrazide

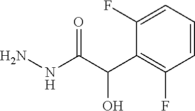 [SMILES: NNC(=O)C(O)c1c(F)cccc1F]

Hydrazine (3.0 mL, 96 mmol) was added to an ethanol (90 mL) solution of methyl 2-(2,6-difluorophenyl)-2-hydroxyacetate (10.8 g, 53 mmol) at RT. The reaction mixture was stirred at 100° C. for 2 h, cooled to RT, concentrated, and used in next step without further purification. LC-MS calculated for C₈H₉F₂N₂O₂ (M+H)⁺: 203.1; found 203.2.

### Step 4: 3-(5-Amino-2-((2,6-difluorophenyl)(hydroxy)methyl)-[1,2,4]triazolo[1,5-c]pyrimidin-7-yl)-2-fluorobenzonitrile

The title compound was prepared using similar procedures as described for Example A5 Step 2, with 3-(2-amino-6-chloropyrimidin-4-yl)-2-fluorobenzonitrile replacing 3-(2-amino-6-chloropyrimidin-4-yl)benzonitrile, and with 2-(2,6-Difluorophenyl)-2-hydroxyacetohydrazide replacing 2-hydroxy-2-phenylacetohydrazide. The two enantiomers were separated by chiral SFC using a Phenomenex (R,R)-Whelk-01 column (21.2×250 mm, 5 m particle size) eluting with an isocratic mobile phase 15% MeOH in CO₂ with a flow rate of 85 mL/minute. The retention times of peak one and peak two were 3.8 min and 5.3 min, respectively. Following concentration, peak two was purified by prep-LCMS (pH=2, MeCN/water with TFA) to give the desired product as a TFA salt. LC-MS calculated for C₁₉H₁₂F₃N₆O (M+H)⁺: 397.1; found 397.1.

### Example A7: Synthesis of 5-Amino-7-(3-cyano-2-fluorophenyl)-2-((2,6-difluorophenyl)(hydroxy)methyl)-[1,2,4]triazolo[1,5-c]pyrimidine-8-carbonitrile (Compound 7)

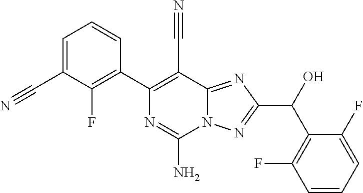 [SMILES: N#Cc1cccc(-c2nc(N)n3nc(C(O)c4c(F)cccc4F)nc3c2C#N)c1F]

### Step 1: 3-(5-Amino-8-bromo-2-((2,6-difluorophenyl)(hydroxy)methyl)-[1,2,4]triazolo[1,5-c]pyrimidin-7-yl)-2-fluorobenzonitrile

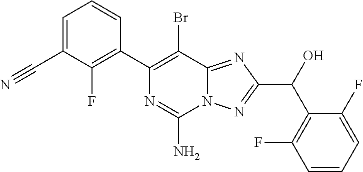 [SMILES: N#Cc1cccc(-c2nc(N)n3nc(C(O)c4c(F)cccc4F)nc3c2Br)c1F]

This compound was prepared using similar procedures as described for Example A1, Step 4, with 3-(5-amino-2-((2,6-difluorophenyl)(hydroxy)methyl)-[1,2,4]triazolo[1,5-c]pyrimidin-7-yl)-2-fluorobenzonitrile (from Example A6) replacing 3-(5-amino-2-(pyridin-2-ylmethyl)-[1,2,4]triazolo[1,5-c]pyrimidin-7-yl)benzonitrile. LCMS calculated for C₁₉H₁₁BrF₃N₆O (M+H)⁺: 475.0; found 475.0.

### Step 2: 5-Amino-7-(3-cyano-2-fluorophenyl)-2-((2,6-difluorophenyl)(hydroxy)methyl)-[1,2,4]triazolo[1,5-c]pyrimidine-8-carbonitrile

A mixture of 3-(5-amino-8-bromo-2-((2,6-difluorophenyl)(hydroxy)methyl)-[1,2,4]triazolo[1,5-c]pyrimidin-7-yl)-2-fluorobenzonitrile (0.12 g, 0.25 mmol), ZnCN₂ (0.060 g, 0.51 mmol) and tBuXPhos Pd G3 (0.020 g, 0.025 mmol) in 1,4-dioxane (0.63 mL) and water (0.63 mL) was purged with N₂ and was stirred at 100° C. for 1 h. After cooling to r.t., the reaction was diluted with saturated NaHCO₃ and the organics were extracted with EtOAc (3×). The combined organics were dried over MgSO₄ and concentrated. The two enantiomers were separated by chiral HPLC using a Phenomenex Lux Celluose-4 column (21.2×250 mm, 5 m particle size) eluting with an isocratic mobile phase 60% EtOH in hexanes with a flow rate of 20 mL/minute. The retention times of peak one and peak two were 4.9 min and 7.2 min, respectively. Following concentration, peak one was purified by preparative LC-MS (pH 2, acetonitrile/water with TFA) to give the desired product as a TFA salt. LC-MS calculated for C₂₀H₁₁F₃N₇O (M+H)⁺: 422.1; found 422.1.

### Example A8: Synthesis of 3-(5-Amino-2-((2-fluoro-6-(((1-methyl-2-oxopyrrolidin-3-yl)amino)methyl)phenyl)(hydroxy)methyl)-[1,2,4]triazolo[1,5-c]pyrimidin-7-yl)-2-fluorobenzonitrile (Compound 8)

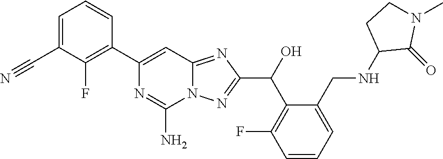 [SMILES: CN1CCC(NCc2cccc(F)c2C(O)c2nc3cc(-c4cccc(C#N)c4F)nc(N)n3n2)C1=O]

### Step 1: Methyl 2-(2-fluoro-6-vinylphenyl)acetate

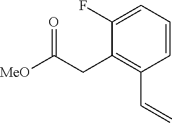

A mixture of methyl 2-(2-bromo-6-fluorophenyl)acetate (6.0 g, 24 mmol), potassium phosphate, tribasic (15.5 g, 73 mmol), palladium(II) acetate (0.55 g, 2.4 mmol), and SPhos (1.0 g, 2.4 mmol) were added to a 500 mL pressure vessel. Next, 4,4,5,5-tetramethyl-2-vinyl-1,3,2-dioxaborolane (6.4 ml, 36 mmol) in dioxane (150 mL) and water (15 mL) was added, the reaction mixture was purged with N₂, and stirred at 80° C. for 16 h. The reaction mixture was then cooled to RT, concentrated, and extracted with EtOAc (×3). The combined organic layers were dried over MgSO₄, concentrated, and purified by column chromatography (0 to 50% EtOAc in DCM). LC-MS calculated for C₁₁H₁₂FO₂ (M+H)⁺: 195.1; found 195.1.

### Step 2: Methyl 2-(2-fluoro-6-vinylphenyl)-2-hydroxyacetate

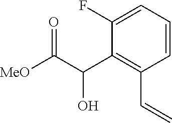

Methyl 2-(2-fluoro-6-vinylphenyl)acetate (2.5 g, 12.9 mmol) was dissolved in THF (130 mL) and cooled to −78° C. LDA (16.7 mL, 16.7 mmol) in THF (1.0 M) was added dropwise, and the resulting solution was stirred at −78° C. for 30 min. Then, 9,9-dimethyltetrahydro-4H-4a,7-methanobenzo[c][1,2]oxazireno[2,3-b]isothiazole 3,3-dioxide (4.7 g, 20.6 mmol) was added dropwise in THF (0.5 M). After 30 min at −78° C., the reaction mixture was warmed to 0° C. and stirred for 1 h. The reaction was quenched with saturated NH₄Cl. The aqueous layer was extracted with DCM (3×). The combined organics were dried over anhydrous Na₂SO₄, filtered, and concentrated under reduced pressure. The crude product was purified by column chromatography eluting with 0 to 50% ethyl acetate in hexanes to afford the desired product. LCMS calculated for C₁₁H₁₁FO₃Na (M+Na)⁺: 233.1; found 233.1.

### Step 3: 2-(2-Fluoro-6-vinylphenyl)-2-hydroxyacetohydrazide

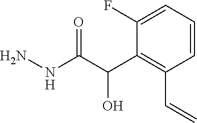

This compound was prepared using similar procedures as described for Example A6, Step 3, with methyl 2-(2-fluoro-6-vinylphenyl)-2-hydroxyacetate replacing methyl 2-(2,6-difluorophenyl)-2-hydroxyacetate. LCMS calculated for C₁₀H₁₂FN₂O₂(M+H)⁺: 211.1; found 211.1.

### Step 4: 3-(5-Amino-2-((2-fluoro-6-vinylphenyl)(hydroxy)methyl)-[1,2,4]triazolo[1,5-c]pyrimidin-7-yl)-2-fluorobenzonitrile

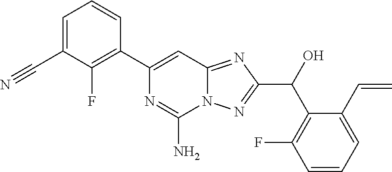 [SMILES: C=Cc1cccc(F)c1C(O)c1nc2cc(-c3cccc(C#N)c3F)nc(N)n2n1]

This compound was prepared using similar procedures as described for Example A6 Step 4, with 2-(2-fluoro-6-vinylphenyl)-2-hydroxyacetohydrazide replacing 2-(2,6-difluorophenyl)-2-hydroxyacetohydrazide. LCMS calculated for C₂₁H₁₅F₂N₆O (M+H)⁺: 405.1; found 405.1.

### Step 5: 3-(5-Amino-2-((2-fluoro-6-formylphenyl)(hydroxy)methyl)-[1,2,4]triazolo[1,5-c]pyrimidin-7-yl)-2-fluorobenzonitrile

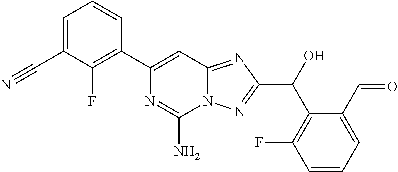 [SMILES: N#Cc1cccc(-c2cc3nc(C(O)c4c(F)cccc4C=O)nn3c(N)n2)c1F]

Osmium tetroxide in water (4% w/w, 0.36 mL, 0.12 mmol) was added to a THF (18 mL) and water (4.6 mL) solution of 3-(5-amino-2-((2-fluoro-6-vinylphenyl)(hydroxy)methyl)-[1,2,4]triazolo[1,5-c]pyrimidin-7-yl)-2-fluorobenzonitrile (930 mg, 2.30 mmol). The reaction mixture was stirred for 5 min at RT and then sodium periodate (2.5 g, 11.5 mmol) was added. After stirring for 1 h, the mixture was diluted with sodium metabisulfite in saturated aq. NaHCO₃ (5% w/w, 20 mL) and extracted with EtOAc (×3). The combined organic layers were dried over MgSO₄ and concentrated under reduced pressure. The crude material was purified by column chromatography eluting with 0 to 100% ethyl acetate in hexanes to afford the desired product. LCMS calculated for C₂₀H₁₃F₂N₆O₂(M+H)⁺: 407.1; found 407.1.

### Step 6: 3-(5-Amino-2-((2-fluoro-6-(((1-methyl-2-oxopyrrolidin-3-yl)amino)methyl)phenyl)(hydroxy)methyl)-[1,2,4]triazolo[1,5-c]pyrimidin-7-yl)-2-fluorobenzonitrile

A solution of 3-amino-1-methylpyrrolidin-2-one (63 mg, 0.55 mmol) and 3-(5-amino-2-((2-fluoro-6-formylphenyl)(hydroxy)methyl)-[1,2,4]triazolo[1,5-c]pyrimidin-7-yl)-2-fluorobenzonitrile (150 mg, 0.37 mmol) was stirred at 40° C. for 2 h in 1,2-dichloroethane (1.9 mL). Then sodium triacetoxyborohydride (160 mg, 0.74 mmol) was added and the reaction mixture was stirred at room temperature for 16 h. The reaction was diluted with saturated NaHCO₃ and the organics were extracted with EtOAc (3×). The combined organics were dried over MgSO₄ and concentrated. The diastereomers were separated by chiral HPLC using a Phenomenex Lux Celluose-4 column (21.2×250 mm, 5 μm particle size) eluting with an isocratic mobile phase 45% EtOH in hexanes with a flow rate of 20 mL/minute. The retention times of peak one and peak two were 14.9 min and 17.5 min, respectively. Following concentration, peak two was further separated by chiral HPLC using a Phenomenex Lux Celluose-1 column (21.2×250 mm, 5 μm particle size) eluting with an isocratic mobile phase 30% EtOH in hexanes with a flow rate of 20 mL/minute. The retention times of peak one and peak two were 11.0 min and 15.5 min, respectively. Following concentration, peak one was purified by preparative LC-MS (pH=2, MeCN/water with TFA) to give the desired product as a TFA salt. LC-MS calculated for C₂₅H₂₃F₂N₈O₂(M+H)⁺: 505.2; found 505.2.

### Example A9: Synthesis of 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile (Compound 9)

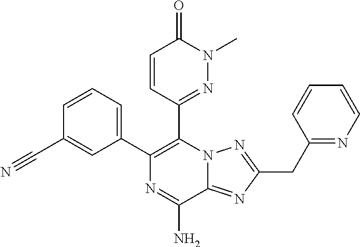 [SMILES: Cn1nc(-c2c(-c3cccc(C#N)c3)nc(N)c3nc(Cc4ccccn4)nn23)ccc1=O]

### Step 1: Methyl 3-bromo-1-(2-(3-cyanophenyl)-2-oxoethyl)-1H-1,2,4-triazole-5-carboxylate

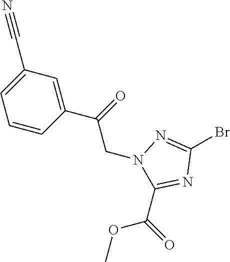 [SMILES: COC(=O)c1nc(Br)nn1CC(=O)c1cccc(C#N)c1]

To a solution of methyl 3-bromo-1H-1,2,4-triazole-5-carboxylate (5.0 g, 24.3 mmol), 3-(2-bromoacetyl)benzonitrile (5.44 g, 24.3 mmol) in DMF (100 mL) was added potassium carbonate (3.35 g, 24.3 mmol). The reaction mixture was stirred at ambient temperature for 2 h. The reaction mixture was then diluted with water and DCM. The organic layer was separated, washed with brine, dried over Na₂SO₄, filtered and concentrated. The resulting residue was purified via flash chromatography to give the desired product as a white solid (5.2 g, 61%). LC-MS calculated for C₁₃H₁₀BrN₄O₃ (M+H)⁺: m/z=349.0; found 349.0.

### Step 2: 3-(2-Bromo-8-oxo-7,8-dihydro-[1,2,4]triazolo[1,5-a]pyrazin-6-yl)benzonitrile

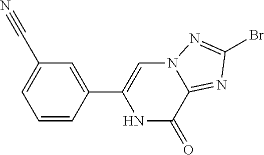 [SMILES: N#Cc1cccc(-c2cn3nc(Br)nc3c(=O)[nH]2)c1]

Methyl 3-bromo-1-(2-(3-cyanophenyl)-2-oxoethyl)-1H-1,2,4-triazole-5-carboxylate (10.5 g, 30.1 mmol) was dissolved in acetic acid (100 mL), and ammonium acetate (23.18 g, 301 mmol) was added. The mixture was stirred at 110° C. for 12 h. After cooling to room temperature, the reaction mixture was diluted with water. The resulting precipitate was collected via filtration, washed with water, and dried under vacuum to afford the product (8.4 g, 88%). LC-MS calculated for C₁₂H₇BrN₅O (M+H)⁺: m/z=316.0; found 316.0.

### Step 3: 3-(2-Bromo-8-chloro-[1,2,4]triazolo[1,5-a]pyrazin-6-yl)benzonitrile

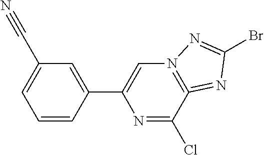

A mixture of 3-(2-bromo-8-oxo-7,8-dihydro-[1,2,4]triazolo[1,5-a]pyrazin-6-yl)benzonitrile (8.4 g, 26.6 mmol) and POCl₃ (49.5 mL, 531 mmol) was stirred at 110° C. overnight. After cooling to room temperature, the reaction mixture was slowly added to a flask containing ice and sodium bicarbonate. The resulting precipitate was collected, washed with water, and dried to afford the product (8.8 g, 99%). LC-MS calculated for C₁₂H₆BrClN₅ (M+H)⁺: m/z=333.9; found 334.0.

### Step 4. 3-(8-(Bis(4-methoxybenzyl)amino)-2-bromo-[1,2,4]triazolo[1,5-a]pyrazin-6-yl)benzonitrile

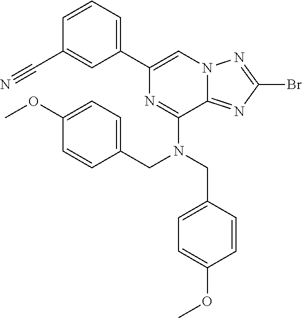 [SMILES: COc1ccc(CN(Cc2ccc(OC)cc2)c2nc(-c3cccc(C#N)c3)cn3nc(Br)nc23)cc1]

A mixture of 3-(2-bromo-8-chloro-[1,2,4]triazolo[1,5-a]pyrazin-6-yl)benzonitrile (8.99 g, 26.9 mmol), bis(4-methoxybenzyl)amine (10.37 g, 40.3 mmol), and DIPEA (9.4 mL, 53.7 mmol) in DMF (134 mL) was stirred at 85° C. overnight. The reaction mixture was cooled to room temperature, and diluted with water. The resulting precipitate was collected via filtration, and dried to afford the product (14.1 g, 94%). LC-MS calculated for C₂₈H₂₄BrN₆O₂ (M+H)⁺: m/z=555.1; found 555.1.

### Step 5: 3-(8-(Bis(4-methoxybenzyl)amino)-2-(pyridin-2-ylmethyl)-[1,2,4]triazolo[1,5-a]pyrazin-6-yl)benzonitrile

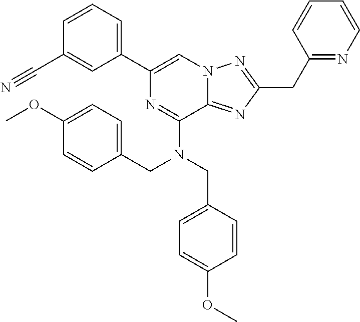 [SMILES: COc1ccc(CN(Cc2ccc(OC)cc2)c2nc(-c3cccc(C#N)c3)cn3nc(Cc4ccccn4)nc23)cc1]

To a solution of 2-methylpyridine (0.050 g, 0.540 mmol) in THF (0.5 mL) was added 2.5 M n-butyllithium (0.216 mL, 0.540 mmol) at −78° C. The resulting solution was stirred at the same temperature for 1 h, before 1.9 M zinc chloride in 2-methyltetrahydrofuran (0.284 mL, 0.540 mmol) was added, and the resulting mixture was stirred at room temperature for 10 min.

A microwave vial charge with 3-(8-(bis(4-methoxybenzyl)amino)-2-bromo-[1,2,4]triazolo[1,5-a]pyrazin-6-yl)benzonitrile (0.15 g, 0.270 mmol), palladium acetate (1.1 mg, 4.7 μmol), and 2′-(dicyclohexylphosphino)-N,N,N′,N′-tetramethylbiphenyl-2,6-diamine (4.1 mg, 9.5 μmol) was evacuated under high vacuum and backfilled with nitrogen. THF (2.0 mL) and toluene (0.5 mL) was then added to the reaction vial. The mixture was cooled to 0° C. and the zinc reagent prepared from previous step was added slowly via a syringe. The reaction mixture was then stirred at 60° C. overnight, cooled to room temperature, and partitioned between ethylacetate and saturated NH₄Cl solution. The layers were separated and the aqueous layer was extracted with ethylacetate. The combined organic layers were washed with water and brine, dried over MgSO₄, and concentrated. The resulting residue was purified via flash chromatography to afford the product (0.11 g, 71%). LC-MS calculated for C₃₄H₃₀N₇O₂(M+H)⁺: m/z=568.2; found 568.3.

### Step 6. 3-(8-Amino-2-(pyridin-2-ylmethyl)-[1,2,4]triazolo[1,5-a]pyrazin-6-yl)benzonitrile

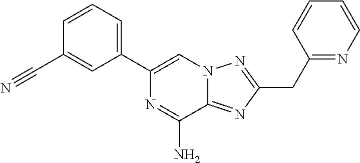 [SMILES: N#Cc1cccc(-c2cn3nc(Cc4ccccn4)nc3c(N)n2)c1]

A mixture of 3-(8-(bis(4-methoxybenzyl)amino)-2-(pyridin-2-ylmethyl)-[1,2,4]triazolo[1,5-a]pyrazin-6-yl)benzonitrile (110 mg, 0.194 mmol) and TFA (746 μL, 9.69 mmol) was stirred at 80° C. for 30 min, cooled to room temperature, and concentrated. The resulting residue was purified via prep-LCMS (pH 2) to give the product as a white solid (TFA salt) (57 mg, 90%). LC-MS calculated for C₁₈H₁₄N₇ (M+H)⁺: m/z=328.1; found 328.1.

### Step 7. 3-(8-Amino-5-bromo-2-(pyridin-2-ylmethyl)-[1,2,4]triazolo[1,5-a]pyrazin-6-yl)benzonitrile

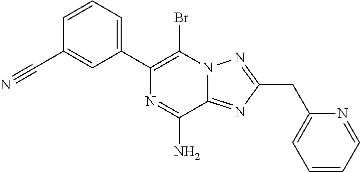 [SMILES: N#Cc1cccc(-c2nc(N)c3nc(Cc4ccccn4)nn3c2Br)c1]

To a solution of 3-(8-amino-2-(pyridin-2-ylmethyl)-[1,2,4]triazolo[1,5-a]pyrazin-6-yl)benzonitrile (TFA salt) (35 mg, 0.079 mmol) in DMF (0.5 mL)/DCM (0.5 mL) was added NBS (14.1 mg, 0.079 mmol). The reaction mixture was then stirred at room temperature for 1 h, and concentrated to afford the crude product, which was used in the next step without further purification. LC-MS calculated for C₁₈H₁₃BrN₇ (M+H)⁺: m/z=406.0; found 406.0.

### Step 8. 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile

A mixture of 6-chloro-2-methylpyridazin-3(2H)-one (30 mg, 0.21 mmol), bis(pinacolato)diboron (53 mg, 0.21 mmol), chloro(2-dicyclohexylphosphino-2′,4′,6′-triisopropyl-1,1′-biphenyl) [2-(2′-amino-1,1′-biphenyl)]palladium(II) (15.7 mg, 0.02 mmol) (XPhos Pd G2) and potassium acetate (61.7 mg, 0.63 mmol) in 1,4-dioxane (1 mL) was stirred at 100° C. for 1 h. 3-(8-Amino-5-bromo-2-(pyridin-2-ylmethyl)-[1,2,4]triazolo[1,5-a]pyrazin-6-yl)benzonitrile (10 mg, 0.025 mmol), cesium carbonate (37.6 mg, 0.116 mmol) and water (0.2 mL) were then added to the reaction mixture. The resulting mixture was heated at 90° C. for 1 h. The mixture was concentrated and purified by preparative LCMS (pH 2, acetonitrile/water with TFA) to afford the desired product as TFA salt. LCMS calculated for C₂₃H₁₈N₉O (M+H)⁺: 436.2; found 436.2.

¹H NMR (500 MHz, DMSO) δ 8.66-8.62 (d, J=5.1 Hz, 1H), 8.09-8.02 (d, J=1.8 Hz, 1H), 7.88-7.85 (t, J=1.8 Hz, 1H), 7.85-7.81 (m, 3H), 7.78-7.72 (d, J=9.6 Hz, 1H), 7.66-7.51 (m, 4H), 7.10-7.06 (d, J=9.6 Hz, 1H), 4.59-4.48 (s, 2H), 3.53-3.43 (s, 3H).

### Example A10: Synthesis of 3-(8-Amino-2-((2,6-difluorophenyl)(hydroxy)methyl)-5-(pyrimidin-4-yl)-[1,2,4]triazolo[1,5-a]pyrazin-6-yl)benzonitrile (Compound 10)

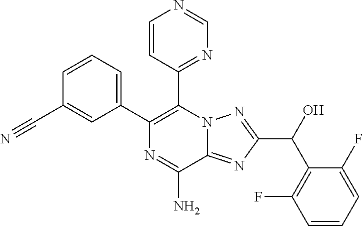 [SMILES: N#Cc1cccc(-c2nc(N)c3nc(C(O)c4c(F)cccc4F)nn3c2-c2ccncn2)c1]

### Step 1: Methyl 3-bromo-1-(2-(3-cyanophenyl)-2-oxoethyl)-1H-1,2,4-triazole-5-carboxylate

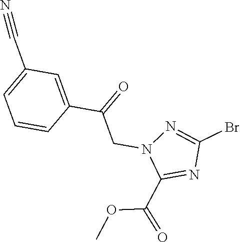 [SMILES: COC(=O)c1nc(Br)nn1CC(=O)c1cccc(C#N)c1]

To a solution of methyl 3-bromo-1H-1,2,4-triazole-5-carboxylate (5.0 g, 24.3 mmol), 3-(2-bromoacetyl)benzonitrile (5.44 g, 24.3 mmol) in DMF (100 mL) was added potassium carbonate (3.35 g, 24.3 mmol). The reaction mixture was stirred at ambient temperature for 2 h. The reaction mixture was then diluted with water and DCM. The organic layer was separated, washed with brine, dried over Na₂SO₄, filtered and concentrated. The resulting residue was purified via flash chromatography to give the desired product as a white solid (5.2 g, 61%). LC-MS calculated for C₁₃H₁₀BrN₄O₃ (M+H)⁺: m/z=349.0; found 349.0.

### Step 2: 3-(2-Bromo-8-oxo-7,8-dihydro-[1,2,4]triazolo[1,5-a]pyrazin-6-yl)benzonitrile

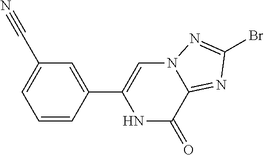 [SMILES: N#Cc1cccc(-c2cn3nc(Br)nc3c(=O)[nH]2)c1]

Methyl 3-bromo-1-(2-(3-cyanophenyl)-2-oxoethyl)-1H-1,2,4-triazole-5-carboxylate (10.5 g, 30.1 mmol) was dissolved in acetic acid (100 mL), and ammonium acetate (23.18 g, 301 mmol) was added. The mixture was stirred at 110° C. for 12 h. After cooling to room temperature, the reaction mixture was diluted with water. The resulting precipitate was collected via filtration, washed with water, and dried under vacuum to afford the product (8.4 g, 88%). LC-MS calculated for C₁₂H₇BrN₅O (M+H)⁺: m/z=316.0; found 316.0.

### Step 3: 3-(2-Bromo-8-chloro-[1,2,4]triazolo[1,5-a]pyrazin-6-yl)benzonitrile

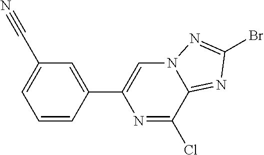 [SMILES: N#Cc1cccc(-c2cn3nc(Br)nc3c(Cl)n2)c1]

A mixture of 3-(2-bromo-8-oxo-7,8-dihydro-[1,2,4]triazolo[1,5-a]pyrazin-6-yl)benzonitrile (8.4 g, 26.6 mmol) and POCl₃ (49.5 mL, 531 mmol) was stirred at 110° C. overnight. After cooling to room temperature, the reaction mixture was slowly added to a flask containing ice and sodium bicarbonate. The resulting precipitate was collected via filtration, washed with water, and dried to afford the product (8.8 g, 99%). LC-MS calculated for C₁₂H₆BrClN₅ (M+H)⁺: m/z=336.0; found 336.0.

### Step 4: 3-(8-(Bis(4-methoxybenzyl)amino)-2-bromo-[1,2,4]triazolo[1,5-a]pyrazin-6-yl)benzonitrile

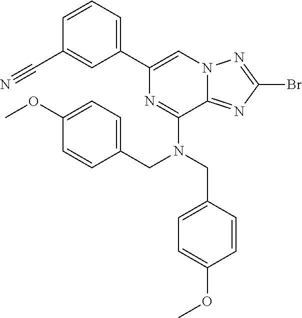 [SMILES: COc1ccc(CN(Cc2ccc(OC)cc2)c2nc(-c3cccc(C#N)c3)cn3nc(Br)nc23)cc1]

A mixture of 3-(2-bromo-8-chloro-[1,2,4]triazolo[1,5-a]pyrazin-6-yl)benzonitrile (8.99 g, 26.9 mmol), bis(4-methoxybenzyl)amine (10.37 g, 40.3 mmol), and DIPEA (9.4 mL, 53.7 mmol) in DMF (134 mL) was stirred at 65° C. overnight. The reaction mixture was cooled to room temperature, and diluted with water. The resulting precipitate was collected via filtration, and dried to afford the product (14.1 g, 94%). LC-MS calculated for C₂₈H₂₄BrN₆O₂ (M+H)⁺: m/z=555.1; found 555.1.

### Step 5: 3-(8-(Bis(4-methoxybenzyl)amino)-2-vinyl-[1,2,4]triazolo[1,5-a]pyrazin-6-yl)benzonitrile

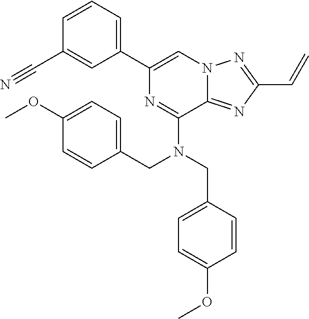 [SMILES: C=Cc1nc2c(N(Cc3ccc(OC)cc3)Cc3ccc(OC)cc3)nc(-c3cccc(C#N)c3)cn2n1]

A mixture of 3-(8-(bis(4-methoxybenzyl)amino)-2-bromo-[1,2,4]triazolo[1,5-a]pyrazin-6-yl)benzonitrile (10.0 g, 18.0 mmol), 4,4,5,5-tetramethyl-2-vinyl-1,3,2-dioxaborolane (3.88 g, 25.2 mmol), potassium phosphate tribasic (9.55 g, 45.0 mmol) and chloro(2-dicyclohexylphosphino-2′,4′,6′-triisopropyl-1,1′-biphenyl)[2-(2′-amino-1,1-biphenyl)]palladium(II) (567 mg, 0.72 mmol) in 1,4-dioxane (200 mL) and water (50 mL) was stirred at 85° C. for 2 hrs. The reaction mixture was cooled to room temperature, and most of 1, 4-dioxane was removed. The resulting precipitate was collected via filtration, washed with water and dried to afford the crude product (9.1 g), which was used in the next step directly. LC-MS calculated for C₃₀H₂₇N₆O₂ (M+H)⁺: m/z=503.2; found 503.1.

### Step 6. 3-(8-(Bis(4-methoxybenzyl)amino)-5-bromo-2-vinyl-[1,2,4]triazolo[1,5-a]pyrazin-6-yl)benzonitrile

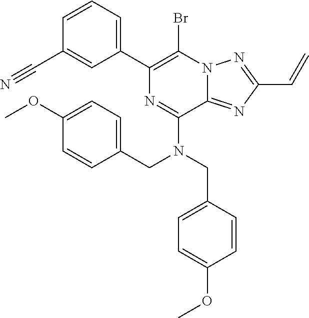 [SMILES: C=Cc1nc2c(N(Cc3ccc(OC)cc3)Cc3ccc(OC)cc3)nc(-c3cccc(C#N)c3)c(Br)n2n1]

To a solution of 3-(8-(bis(4-methoxybenzyl)amino)-2-vinyl-[1,2,4]triazolo[1,5-a]pyrazin-6-yl)benzonitrile (717 mg, 1.43 mmol) in 10 mL of dichloromethane, 1-bromopyrrolidine-2,5-dione (254 mg, 1.43 mmol) was added at 0° C. The resulting mixture was stirred for 4 hrs, and directly purified by a silica gel column to afford the desired product (780 mg, 94%). LC-MS calculated for C₃₀H₂₆BrN₆O₂ (M+H)⁺: m/z=581.1; found 581.2.

### Step 7: 3-(8-(Bis(4-methoxybenzyl)amino)-5-(pyrimidin-4-yl)-2-vinyl-[1,2,4]triazolo[1,5-a]pyrazin-6-yl)benzonitrile

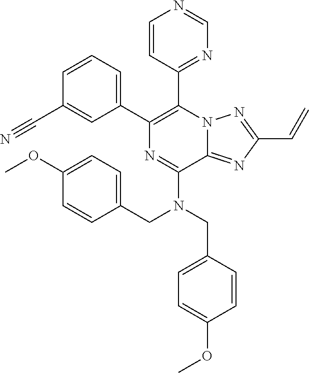 [SMILES: C=Cc1nc2c(N(Cc3ccc(OC)cc3)Cc3ccc(OC)cc3)nc(-c3cccc(C#N)c3)c(-c3ccncn3)n2n1]

A mixture of 3-(8-(bis(4-methoxybenzyl)amino)-5-bromo-2-vinyl-[1,2,4]triazolo[1,5-a]pyrazin-6-yl)benzonitrile (260 mg, 0.45 mmol), 4-(tributylstannyl)pyrimidine (215 mg, 0.58 mmol), lithium chloride (28.4 mg, 0.67 mmol), copper(I) chloride (67 mg, 0.67 mmol), and Tetrakis(triphenylphosphine)palladium(0) (52 mg, 0.045 mmol) in THF (5 mL) was stirred at 90° C. for 45 mins. The reaction mixture was quenched with water and extracted with dichloromethane. The combined organic layers were concentrated, and purified by a silica gel column to afford the desired product (176 mg, 67%). LC-MS calculated for C₃₄H₂₉N₈O₂ (M+H)⁺: m/z=581.2; found 581.1.

### Step 8: 3-(8-(Bis(4-methoxybenzyl)amino)-2-formyl-5-(pyrimidin-4-yl)-[1,2,4]triazolo[1,5-a]pyrazin-6-yl)benzonitrile

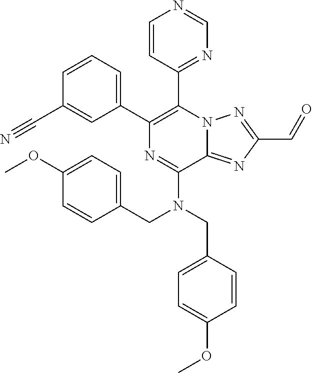 [SMILES: COc1ccc(CN(Cc2ccc(OC)cc2)c2nc(-c3cccc(C#N)c3)c(-c3ccncn3)n3nc(C=O)nc23)cc1]

A mixture of 3-(8-(bis(4-methoxybenzyl)amino)-5-(pyrimidin-4-yl)-2-vinyl-[1,2,4]triazolo[1,5-a]pyrazin-6-yl)benzonitrile (176 mg, 0.3 mmol), osmium(VIII) oxide (3 mg in 0.3 mL water, 0.015 mmol), and sodium periodate (292 mg, 1.36 mmol) in THF/water (1:1, 6 mL) was stirred at 65° C. for 1 h. The reaction mixture was cooled to 15 room temperature, and extracted with dichloromethane. The combined organic layers were concentrated, and purified by silica gel column to afford the desired product (130 mg, 74%). LC-MS calculated for C₃₃H₂₇N₈O₃ (M+H)⁺: m/z=583.2; found 583.2.

### Step 9: 3-(8-Amino-2-((2,6-difluorophenyl)(hydroxy)methyl)-5-(pyrimidin-4-yl)-[1,2,4]triazolo[1,5-a]pyrazin-6-yl)benzonitrile

Preparation of the Grignard reagent: To a solution of 1,3-difluoro-2-iodobenzene (142 mg, 0.6 mmol) in tetrahydrofuran (1 mL), isopropylmagnesium chloride solution (296 μl, 2 M) was added at −10° C. The resulting mixture was stirred for 1 h, and used directly in the following step.

To a solution of 3-(8-(bis(4-methoxybenzyl)amino)-2-formyl-5-(pyrimidin-4-yl)-[1,2,4]triazolo[1,5-a]pyrazin-6-yl)benzonitrile (120 mg, 0.2 mmol) in THF (2 mL), the freshly prepared Grignard reagent from previous step was added at −10° C. The reaction mixture was stirred for 30 min, quenched with ammonium chloride solution (4 mL), and extracted with dichloromethane. The combined organic layers were concentrated under vacuum. The resulting material was dissolved in TFA (5 mL), and stirred at 80° C. for 20 min. The reaction mixture was then cooled to room temperature, concentrated, and basified by adding aqeuous NaHCO₃ solution.

The crude material was directly purified by a silica gel column to afford the desired product (60 mg, 64%) as a racemic mixture. The product was then separated with chiral HPLC using a chiral column (Phenomenex Lux 5 um Cellulose-4, 21.2×250 mm) and 75% EtOH in hexanes (20 mL/min) solvent system.

Peak 2 was isolated, and further purified via preparative LC/MS (pH=2, acetonitrile/water with TFA) to give the desired product as a TFA salt. LC-MS calculated for C₂₃H₁₅F₂N₈O (M+H)⁺: m/z=457.1; found 457.0.

¹H NMR (600 MHz, DMSO-d₆) δ 9.14 (d, J=1.3 Hz, 1H), 8.95 (d, J=5.2 Hz, 1H), 7.90 (dd, J=5.2, 1.4 Hz, 1H), 7.88 (s, 1H), 7.78 (dt, J=7.6, 1.4 Hz, 1H), 7.74 (t, J=1.4 Hz, 1H), 7.54 (dt, J=7.9, 1.3 Hz, 1H), 7.51-7.40 (m, 2H), 7.09 (t, J=8.4 Hz, 2H), 6.27 (s, 1H).

### Example A11: Synthesis of 3-(8-amino-2-(amino(2,6-difluorophenyl)methyl)-5-(4-methyloxazol-5-yl)-[1,2,4]triazolo[1,5-a]pyrazin-6-yl)benzonitrile (Compound 11)

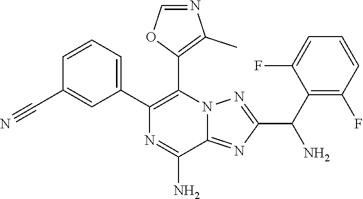 [SMILES: Cc1ncoc1-c1c(-c2cccc(C#N)c2)nc(N)c2nc(C(N)c3c(F)cccc3F)nn12]

### Step 1: 3-(8-(Bis(4-methoxybenzyl)amino)-5-bromo-2-vinyl-[1,2,4]triazolo[1,5-a]pyrazin-6-yl)benzonitrile

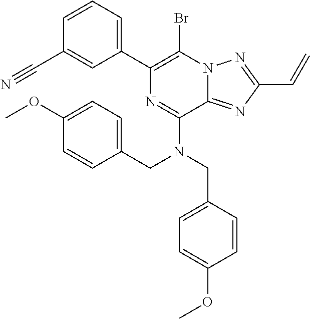 [SMILES: C=Cc1nc2c(N(Cc3ccc(OC)cc3)Cc3ccc(OC)cc3)nc(-c3cccc(C#N)c3)c(Br)n2n1]

To a solution of 3-(8-(bis(4-methoxybenzyl)amino)-2-vinyl-[1,2,4]triazolo[1,5-a]pyrazin-6-yl)benzonitrile (Example A10, Step 5; 241 mg, 0.48 mmol) in DCM (5 mL) was added NBS (84.6 mg, 0.48 mmol). The reaction mixture was then stirred at room temperature for 1 h, and concentrated to afford the crude product, which was used in the next step without further purification. LC-MS calculated for C₃₀H₂₆BrN₆O₂ (M+H)⁺: m/z=581.1; found 581.1.

### Step 2: 3-(8-(bis(4-methoxybenzyl)amino)-5-bromo-2-formyl-[1,2,4]triazolo[1,5-a]pyrazin-6-yl)benzonitrile

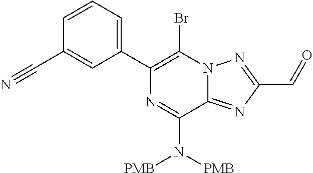

A mixture of 3-(8-(bis(4-methoxybenzyl)amino)-5-bromo-2-vinyl-[1,2,4]triazolo[1,5-a]pyrazin-6-yl)benzonitrile (174 mg, 0.3 mmol), osmium(VIII) oxide (3 mg in 0.3 mL water, 0.015 mmol), and sodium periodate (292 mg, 1.36 mmol) in THF/water (1:1, 6 mL) was stirred at 65° C. for 1 h. The reaction mixture was cooled to room temperature, and extracted with dichloromethane. The combined organic layers were concentrated, and purified by silica gel column to afford the desired product. LC-MS calculated for C₂₉H₂₄N₆O₃Br (M+H)⁺: m/z=583.1; found 583.1.

### Step 3: 3-(8-(bis(4-methoxybenzyl)amino)-5-bromo-2-((2,6-difluorophenyl)(hydroxy)methyl)-[1,2,4]triazolo[1,5-a]pyrazin-6-yl)benzonitrile

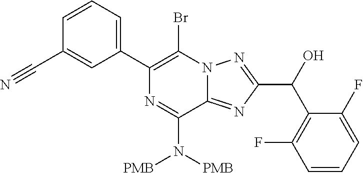

Preparation of the Grignard reagent: To a solution of 1,3-difluoro-2-iodobenzene (142 mg, 0.6 mmol) in tetrahydrofuran (1 mL), isopropylmagnesium chloride solution (296 μl, 2 M) was added at −10° C. The resulting mixture was stirred for 1 h, and used directly in the following step.

To a solution of 3-(8-(bis(4-methoxybenzyl)amino)-5-bromo-2-formyl-[1,2,4]triazolo[1,5-a]pyrazin-6-yl)benzonitrile (120 mg, 0.2 mmol) in THF (2 mL), the freshly prepared Grignard reagent from previous step was added at −10° C. The reaction mixture was stirred for 30 min, quenched with ammonium chloride solution (4 mL), and extracted with dichloromethane. The combined organic layers were concentrated under vacuum and purified by a silica gel column to afford the desired product as a racemic mixture. LC-MS calculated for C₃₅H₂₈N₆O₃BrF₂ (M+H)⁺: m/z=697.1; found 697.1.

### Step 4: 3-(8-(bis(4-methoxybenzyl)amino)-2-((2,6-difluorophenyl)(hydroxy)methyl)-5-(4-methyloxazol-5-yl)-[1,2,4]triazolo[1,5-a]pyrazin-6-yl)benzonitrile

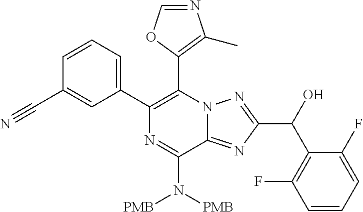

A mixture of 3-(8-(bis(4-methoxybenzyl)amino)-5-bromo-2-((2,6-difluorophenyl)(hydroxy)methyl)-[1,2,4]triazolo[1,5-a]pyrazin-6-yl)benzonitrile (382 mg, 0.55 mmol), 4-methyl-5-(4,4,5,5-tetramethyl-1,3,2-dioxaborolan-2-yl)oxazole (137 mg, 0.65 mmol), dicyclohexyl(2′,4′,6′-triisopropylbiphenyl-2-yl)phosphine-(2′-aminobiphenyl-2-yl)(chloro)palladium (1:1) (17 mg, 21.6 μmol) and Cs₂CO₃ (356 mg, 1.09 mmol) in 1,4-dioxane (2 mL) and water (200 μl) was purged with N₂ and heated at 95° C. for 7 h. The mixture was concentrated and purified via flash chromatography to afford the desired product as a colorless oil. LCMS calculated for C₃₉H₃₂N₇O₄F₂(M+H)⁺: 700.2; found 700.2.

### Step 5: 3-(8-(bis(4-methoxybenzyl)amino)-2-(chloro(2,6-difluorophenyl)methyl)-5-(4-methyloxazol-5-yl)-[1,2,4]triazolo[1,5-a]pyrazin-6-yl)benzonitrile

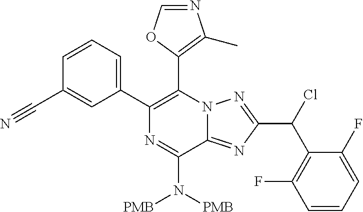

To a solution of 3-(8-(bis(4-methoxybenzyl)amino)-2-((2,6-difluorophenyl)(hydroxy)methyl)-5-(4-methyloxazol-5-yl)-[1,2,4]triazolo[1,5-a]pyrazin-6-yl)benzonitrile (201 mg, 0.29 mmol) in 2 mL of dichloromethane, thionyl chloride (105 μl, 1.435 mmol) was added at rt. The resulting mixture was stirred for 4 h, concentrated and used in next step without any further purification. LC-MS calculated for C₃₉H₃₁N₇O₃ClF₂ (M+H)⁺: m/z=718.2; found 718.2.

### Step 6: 3-(8-amino-2-(amino(2,6-difluorophenyl)methyl)-5-(4-methyloxazol-5-yl)-[1,2,4]triazolo[1,5-a]pyrazin-6-yl)benzonitrile

To a solution of 3-(8-(bis(4-methoxybenzyl)amino)-2-(chloro(2,6-difluorophenyl)methyl)-5-(4-methyloxazol-5-yl)-[1,2,4]triazolo[1,5-a]pyrazin-6-yl)benzonitrile (40 mg, 0.084 mmol) in 1 mL of DMSO was added ammonia solution (1 mL). The mixture was heated with microwave condition at 100° C. for 10 h before diluted with water and extracted with EtOAc. The combined organic layers were washed with water and brine, dried over MgSO₄, and concentrated. The resulting residue was dissolved in TFA (1 mL), and stirred at 80° C. for 20 min. The reaction mixture was then cooled to room temperature, concentrated, and basified by adding aq. NaHCO₃ solution. The crude material was directly purified by a silica gel column to afford the desired product as a racemic mixture. The product was then separated with chiral HPLC using a chiral column (AM-1) and 45% EtOH in hexanes (20 mL/min) solvent system. Peak 1 was isolated, and further purified via preparative LC/MS (pH=2, acetonitrile/water with TFA) to give the desired product as a TFA salt. LC-MS calculated for C₂₃H₁₇F₂N₈O (M+H)⁺: m/z=459.1; found 459.0.

### Example A12: Synthesis of 3-(8-amino-2-((2,6-difluorophenyl)(hydroxy)methyl)-5-(2,6-dimethylpyridin-4-yl)-[1,2,4]triazolo[1,5-a]pyrazin-6-yl)benzonitrile (Compound 12)

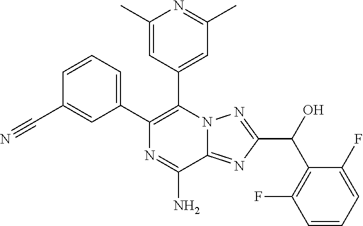 [SMILES: Cc1cc(-c2c(-c3cccc(C#N)c3)nc(N)c3nc(C(O)c4c(F)cccc4F)nn23)cc(C)n1]

To a solution of 3-(8-(bis(4-methoxybenzyl)amino)-5-bromo-2-((2,6-difluorophenyl)(hydroxy)methyl)-[1,2,4]triazolo[1,5-a]pyrazin-6-yl)benzonitrile (Example A11, Step 3; 0.518 g, 0.638 mmol), 2,6-dimethyl-4-(4,4,5,5-tetramethyl-1,3,2-dioxaborolan-2-yl)pyridine (0.346 g, 1.48 mmol), and dicyclohexyl(2′,4′,6′-triisopropylbiphenyl-2-yl)phosphine-(2′-aminobiphenyl-2-yl)(chloro)palladium (1:1) (0.058 g, 0.074 mmol) in dioxane (3.0 mL) and water (0.60 mL) was added potassium phosphate tribasic (0.472 g, 2.23 mmol). The reaction mixture was stirred at 90° C. for 1 h. The reaction mixture was then diluted with water and DCM. The layers were separated, the aqueous layer was extracted with DCM, and the combined organic fractions were dried over MgSO₄, filtered and concentrated. The crude material was dissolved in TFA (5 mL) and heated to 80° C. for 20 minutes. The reaction mixture was then cooled to room temperature, concentrated, and basified by adding aqueous NaHCO₃ solution. The crude material was directly purified by a silica gel column to afford the desired product (257 mg, 72%) as a racemic mixture.

The product was then separated with chiral HPLC using a chiral column (Phenomenex Lux Sum Cellulose-2, 21.1×250 mm) and 35% EtOH in Hexanes (20 mL/min) solvent system. Peak 2 was isolated, and further purified using preparative LC/MS (pH=2, acetonitrile/water with TFA) to give the desired product as a TFA salt. LC-MS calculated for C₂₆H₂₀F₂N₇O (M+H)⁺: m/z=484.2; found 484.2. ¹H NMR (500 MHz, DMSO-d₆) δ 7.92 (s, 2H), 7.85 (s, 1H), 7.83 (d, J=7.6 Hz, Ili), 7.56 (d, J=8.0 Hz, 1H), 7.53-7.40 (m, 4H), 7.10 (t, J=8.4 Hz, 2H), 6.27 (s, 1H), 2.51 (s, 6H).

### Example A13: Synthesis of 3-(4-amino-2-(pyridin-2-ylmethyl)-7-(pyrimidin-4-yl)-2H-[1,2,3]triazolo[4,5-c]pyridin-6-yl)benzonitrile (Compound 13)

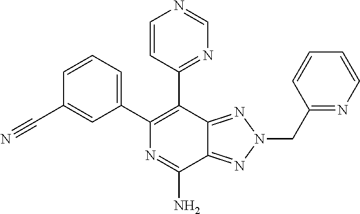 [SMILES: N#Cc1cccc(-c2nc(N)c3nn(Cc4ccccn4)nc3c2-c2ccncn2)c1]

### Step 1. 4,6-dichloro-3H-[1,2,3]triazolo[4,5-c]pyridine

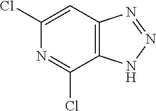 [SMILES: Clc1cc2nn[nH]c2c(Cl)n1]

A solution of NaNO₂ (3.88 g, 56.2 mmol) in water (3 mL) was added to a solution of 2,6-dichloropyridine-3,4-diamine (10 g, 56 mmol) in hydrochloric Acid, 37% (5 mL) at 0° C. The solution was stirred for 30 min. Water (20 mL) was added and the white precipitate was filtered, washed with water, and dried to give the desired product. LC-MS calculated for C₅H₃C₁₂N₄: 189.0 (M+H)⁺; found: 189.0 (M+H)⁺.

### Step 2. 6-chloro-N-(2,4-dimethoxybenzyl)-3H-[1,2,3]triazolo[4,5-c]pyridin-4-amine

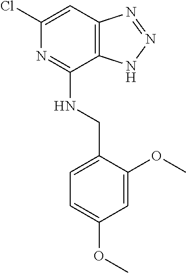 [SMILES: COc1ccc(CNc2nc(Cl)cc3nn[nH]c23)c(OC)c1]

The mixture of 4,6-dichloro-3H-[1,2,3]triazolo[4,5-c]pyridine (600 mg, 3.17 mmol), (2,4-dimethoxyphenyl)methanamine (0.53 mL, 3.49 mmol) and triethylamine (0.53 mL, 3.81 mmol) in 1,4-dioxane (10 mL) was stirred at 110° C. for 3 days. Direct purification on silica gel column afforded the desired product (875 mg, 86%). LC-MS calculated for C₁₄H₁₅ClN₅O₂: 320.1 (M+H)⁺; found: 320.3 (M+H)⁺.

### Step 3. 6-chloro-N-(2,4-dimethoxybenzyl)-2-(pyridin-2-ylmethyl)-2H-[1,2,3]triazolo[4,5-c]pyridin-4-amine

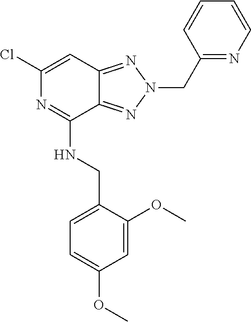 [SMILES: COc1ccc(CNc2nc(Cl)cc3nn(Cc4ccccn4)nc23)c(OC)c1]

The mixture of 6-chloro-N-(2,4-dimethoxybenzyl)-3H-[1,2,3]triazolo[4,5-c]pyridin-4-amine (875 mg, 2.74 mmol), pyridin-2-ylmethanol (0.317 mL, 3.28 mmol) and triphenylphosphine (1436 mg, 5.47 mmol) in DCM (20 mL) was added diisopropyl azodicarboxylate (0.647 mL, 3.28 mmol) at 0° C. The resulting mixture was stirred at 0° C. for 1 h. Direct purification on silica gel column afforded the desired product (375 mg, 33.4% yield). LC-MS calculated for C₂₀H₂₀ClN₆O₂: 411.1 (M+H)⁺; found: 411.2 (M+H)⁺.

### Step 4. 3-(4-((2,4-dimethoxybenzyl)amino)-2-(pyridin-2-ylmethyl)-2H-[1,2,3]triazolo[4,5-c]pyridin-6-yl)benzonitrile

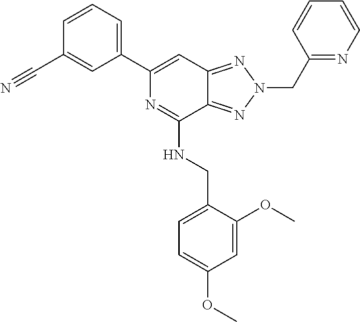 [SMILES: COc1ccc(CNc2nc(-c3cccc(C#N)c3)cc3nn(Cc4ccccn4)nc23)c(OC)c1]

To the mixture of 6-chloro-N-(2,4-dimethoxybenzyl)-2-(pyridin-2-ylmethyl)-2H-[1,2,3]triazolo[4,5-c]pyridin-4-amine (375 mg, 0.913 mmol) and (3-cyanophenyl)boronic acid (268 mg, 1.825 mmol) in 1,4-dioxane (10 mL) and water (1.00 mL) was added cesium carbonate (595 mg, 1.825 mmol). The resulting mixture was purged with N₂ and then chloro(2-dicyclohexylphosphino-2′,4′,6′-triisopropyl-1,1′-biphenyl)[2-(2′-amino-1,1′-biphenyl)]palladium(II) (71.8 mg, 0.091 mmol) was added.

The reaction mixture was stirred at 120° C. under microwave irradiation for 90 min. The reaction was quenched with 20 mL of ethyl acetate and 20 mL of water. The organic phase was separated and the aqueous solution was extracted with ethyl acetate twice. The combined extracts were dried over Na₂SO₄, filtered and evaporated under reduced pressure. The residue was purified on silica gel column to afford the desired product (300 mg, 68.9%). LC-MS calculated for C₂₇H₂₄N₇O₂: 478.2 (M+H)⁺; found: 478.3 (M+H)⁺.

### Step 5. 3-(4-amino-2-(pyridin-2-ylmethyl)-2H-[1,2,3]triazolo[4,5-c]pyridin-6-yl)benzonitrile

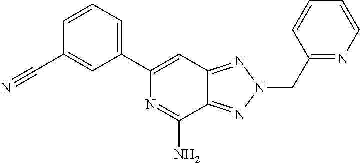 [SMILES: N#Cc1cccc(-c2cc3nn(Cc4ccccn4)nc3c(N)n2)c1]

The solution of 3-(4-((2,4-dimethoxybenzyl)amino)-2-(pyridin-2-ylmethyl)-2H-[1,2,3]triazolo[4,5-c]pyridin-6-yl)benzonitrile (300.3 mg, 0.629 mmol) in TFA (5 mL) was stirred at 100° C. for 30 min. TFA was evaporated under reduced pressure and then 20 mL of saturated NaHCO₃ aqueous solution and 20 mL of ethyl acetate were added. The organic phase was separated and the aqueous solution was extracted with ethyl acetate twice. The combined extracts were dried over Na₂SO₄, filtered and evaporated under reduced pressure. The residue was purified on silica gel column to afford the desired product (175 mg, 85%). LC-MS calculated for C₁₈H₁₄N₇: 328.1 (M+H)⁺; found: 328.2 (M+H)⁺.

### Step 6. 3-(4-amino-7-bromo-2-(pyridin-2-ylmethyl)-2H-[1,2,3]triazolo[4,5-c]pyridin-6-yl)benzonitrile

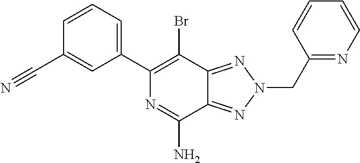 [SMILES: N#Cc1cccc(-c2nc(N)c3nn(Cc4ccccn4)nc3c2Br)c1]

The mixture of 3-(4-amino-2-(pyridin-2-ylmethyl)-2H-[1,2,3]triazolo[4,5-c]pyridin-6-yl)benzonitrile (175 mg, 0.535 mmol) and 1-bromopyrrolidine-2,5-dione (100 mg, 0.561 mmol) in THF (10 mL) was stirred at 0° C. for 30 min and then quenched with saturated NaHCO₃ aqueous solution. The organic phase was separated, dried over Na₂SO₄, filtered and evaporated under reduced pressure. The resulting residue was purified on silica gel column to afforded the desired product (135 mg, 62.2%). LC-MS calculated for C₁₈H₁₃BrN₇: 406.0 (M+H)⁺ and 408.0 (M+H)⁺; found: 406.1 (M+H)⁺ and 408.2 (M+H)⁺.

### Step 7. 3-(4-amino-2-(pyridin-2-ylmethyl)-7-(pyrimidin-4-yl)-2H-[1,2,3]triazolo[4,5-c]pyridin-6-yl)benzonitrile

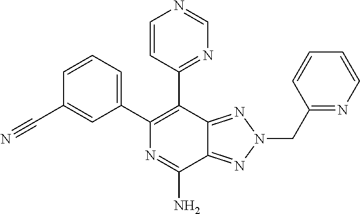 [SMILES: N#Cc1cccc(-c2nc(N)c3nn(Cc4ccccn4)nc3c2-c2ccncn2)c1]

A mixture of 3-(4-amino-7-bromo-2-(pyridin-2-ylmethyl)-2H-[1,2,3]triazolo[4,5-c]pyridin-6-yl)benzonitrile (182 mg, 0.448 mmol), 4-(tributylstannyl)pyrimidine (496 mg, 1.344 mmol), and copper(I) chloride (53.2 mg, 0.538 mmol), lithium chloride (22.79 mg, 0.538 mmol) and tetrakis(triphenylphosphine)palladium(0) (51.8 mg, 0.045 mmol) in THF (1 ml) was first purged with N₂, and then heated and stirred at 90° C. for 2 h. The reaction was diluted with methanol and purified with prep-LCMS (pH=2) to give the desired product. LC-MS calculated for C₂₂H₁₆N₉: 406.2 (M+H)⁺; found: 406.2 (M+H)⁺.

### Example A14: Synthesis of 3-(4-amino-2-((3-fluoropyridin-2-yl)methyl)-7-(pyrimidin-4-yl)-2H-[1,2,3]triazolo[4,5-c]pyridin-6-yl)benzonitrile (Compound 14)

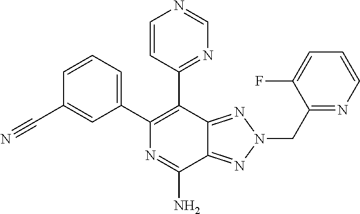 [SMILES: N#Cc1cccc(-c2nc(N)c3nn(Cc4ncccc4F)nc3c2-c2ccncn2)c1]

### Step 1. 6-chloro-N-(2,4-dimethoxybenzyl)-2-((3-fluoropyridin-2-yl)methyl)-2H-[1,2,3]triazolo[4,5-c]pyridin-4-amine

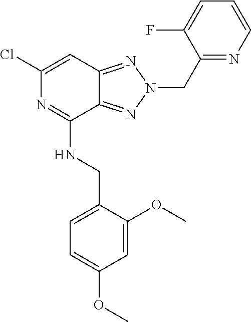 [SMILES: COc1ccc(CNc2nc(Cl)cc3nn(Cc4ncccc4F)nc23)c(OC)c1]

To the mixture of 6-chloro-N-(2,4-dimethoxybenzyl)-3H-[1,2,3]triazolo[4,5-c]pyridin-4-amine (Example A13, Step 2; 1000 mg, 3.13 mmol), (3-fluoropyridin-2-yl)methanol (477 mg, 3.75 mmol) and triphenylphosphine (1641 mg, 6.25 mmol) in DCM (1.7 mL) was added diisopropyl azodicarboxylate (739 μl, 3.75 mmol) at 0° C. The reaction mixture was stirred at 0° C. for 1 h. Direct purification on silica gel column afforded the desired product (433 mg, 32%). LC-MS calculated for C₂₀H₁₉ClFN₆O₂: 429.1 (M+H)⁺; found: 429.3 (M+H)⁺.

### Step 2. 3-(4-((2,4-dimethoxybenzyl)amino)-2-((3-fluoropyridin-2-yl)methyl)-2H-[1,2,3]triazolo[4,5-c]pyridin-6-yl)benzonitrile

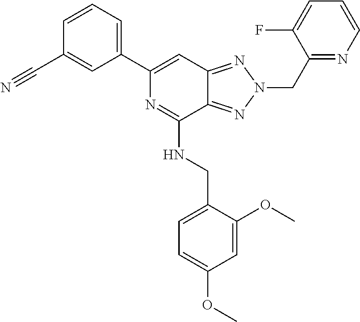 [SMILES: COc1ccc(CNc2nc(-c3cccc(C#N)c3)cc3nn(Cc4ncccc4F)nc23)c(OC)c1]

Cesium carbonate (658 mg, 2.019 mmol) was added to the mixture of 6-chloro-N-(2,4-dimethoxybenzyl)-2-((3-fluoropyridin-2-yl)methyl)-2H-[1,2,3]triazolo[4,5-c]pyridin-4-amine (433 mg, 1.010 mmol) and (3-cyanophenyl)boronic acid (297 mg, 2.019 mmol) in 1,4-dioxane (10.0 mL) and water (1.0 mL). The resulting mixture was sparged with N₂ for 2 min and (SP-4-4)-[2′-Amino[1,1′-biphenyl]-2-yl]chloro[dicyclohexyl[2′,4′,6′-tris(1-methylethyl) [1,1′-biphenyl]-2-yl]phosphine]palladium (79 mg, 0.101 mmol) was added. The reaction mixture was stirred at 120° C. for 1.5 h under microwave irradiation. The reaction was quenched with 20 mL of ethyl acetate and 20 mL of water. The organic phase was separated and the aqueous solution was extracted with ethyl acetate twice. The combined extracts were dried over Na₂SO₄, filtered and evaporated under reduced pressure. The residue was purified on silica gel column to afford the desired product (357 mg, 71%). LC-MS calculated for C₂₇H₂₃FN₇O₂: 496.2 (M+H)⁺; found: 496.3 (M+H)⁺.

### Step 3. 3-(4-amino-2-((3-fluoropyridin-2-yl)methyl)-2H-[1,2,3]triazolo[4,5-c]pyridin-6-yl)benzonitrile

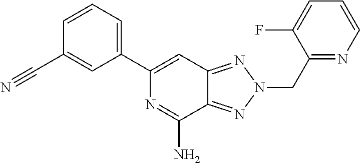 [SMILES: N#Cc1cccc(-c2cc3nn(Cc4ncccc4F)nc3c(N)n2)c1]

The solution of 3-(4-((2,4-dimethoxybenzyl)amino)-2-((3-fluoropyridin-2-yl)methyl)-2H-[1,2,3]triazolo[4,5-c]pyridin-6-yl)benzonitrile (357.3 mg, 0.721 mmol) in TFA (5 mL) was stirred at 100° C. for 1 h. TFA was evaporated under reduced pressure and then 20 mL of saturated NaHCO₃ aqueous solution and 20 mL of ethyl acetate were added. The organic phase was separated and the aqueous solution was extracted with ethyl acetate twice. The combined extracts were dried over Na₂SO₄, filtered and evaporated under reduced pressure. The residue was purified on silica gel column to afford the desired product (213 mg, 61%). LC-MS m/z calculated for C₁₈H₁₃FN₇: 346.1 (M+H)⁺; found: 346.3 (M+H)⁺.

### Step 4. 3-(4-amino-7-bromo-2-((3-fluoropyridin-2-yl)methyl)-2H-[1,2,3]triazolo[4,5-c]pyridin-6-yl)benzonitrile

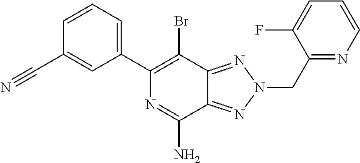 [SMILES: N#Cc1cccc(-c2nc(N)c3nn(Cc4ncccc4F)nc3c2Br)c1]

The mixture of 3-(4-amino-2-((3-fluoropyridin-2-yl)methyl)-2H-[1,2,3]triazolo[4,5-c]pyridin-6-yl)benzonitrile (213 mg, 0.617 mmol) and 1-bromopyrrolidine-2,5-dione (220 mg, 1.234 mmol) in THF (5 mL) was stirred at 0° C. for 1 h. Direct purification on silica gel afforded the desired product (175 mg, 67%). LC-MS calculated for C₁₈H₁₂BrFN₇: 424.0 (M+H)⁺ and 426.0 (M+H)⁺; found: 424.3 (M+H)⁺ and 426.3 (M+H)⁺.

### Step 5. 3-(4-amino-2-((3-fluoropyridin-2-yl)methyl)-7-(pyrimidin-4-yl)-2H-[1,2,3]triazolo[4,5-c]pyridin-6-yl)benzonitrile

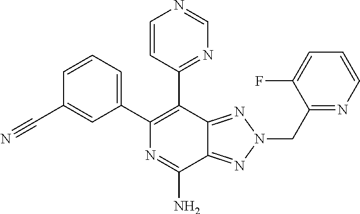 [SMILES: N#Cc1cccc(-c2nc(N)c3nn(Cc4ncccc4F)nc3c2-c2ccncn2)c1]

The mixture of 3-(4-amino-7-bromo-2-((3-fluoropyridin-2-yl)methyl)-2H-[1,2,3]triazolo[4,5-c]pyridin-6-yl)benzonitrile (220 mg, 0.519 mmol), 4-(tributylstannyl)pyrimidine (383 mg, 1.037 mmol), and copper(I) chloride (61.6 mg, 0.622 mmol), lithium chloride (26.4 mg, 0.622 mmol) and tetrakis(triphenylphosphine)palladium(0) (59.9 mg, 0.052 mmol) in THF (1 ml) was first purged with N₂, and then heated and stirred at 90° C. for 2 h. The reaction was diluted with methanol and purified with prep-LCMS (pH=2) to give the desired product. LC-MS calculated for C₂₂H₁₅FN₉: 424.1 (M+H)⁺; found: 424.3 (M+H)⁺. ¹H NMR (500 MHz, DMSO-d₆) ppm 8.98 (s, 1H), 8.77 (d, J=5.02 Hz, 1H), 8.38 (dd, J₁=4.60 Hz, J₂=1.32 Hz, 1H), 7.90-8.30 (bs, 2H), 7.76-7.89 (m, 3H), 7.66 (dd, J₁=5.25 Hz, J₂=1.25 Hz, 1H), 7.45-7.58 (m, 3H), 6.25 (s, 2H).

### Example A15: Synthesis of 3-(4-amino-2-((3-fluoropyridin-2-yl)methyl)-7-(pyridin-4-yl)-2H-[1,2,3]triazolo[4,5-c]pyridin-6-yl)benzonitrile (Compound 15)

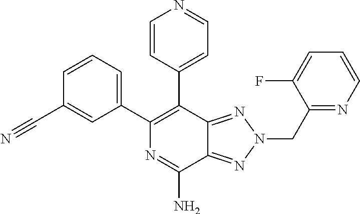 [SMILES: N#Cc1cccc(-c2nc(N)c3nn(Cc4ncccc4F)nc3c2-c2ccncc2)c1]

Cesium carbonate (46.1 mg, 0.141 mmol) was added to a mixture of 3-(4-amino-7-bromo-2-((3-fluoropyridin-2-yl)methyl)-2H-[1,2,3]triazolo[4,5-c]pyridin-6-yl)benzonitrile (30 mg, 0.071 mmol) and pyridin-4-ylboronic acid (17.38 mg, 0.141 mmol) in 1,4-dioxane (2 mL) and water (0.2 mL). The resulting mixture was sparged with N₂ for 2 min and chloro(2-dicyclohexylphosphino-2′,4′,6′-triisopropyl-1,1′-biphenyl)[2-(2′-amino-1,1′-biphenyl)]palladium(II) (5.56 mg, 7.07 μmol) was added. The reaction mixture was stirred at 120° C. for 1.5 h under microwave irradiation. The reaction mixture was diluted with methanol. Direct purification on prep. HPLC afforded the desired product. LC-MS calculated for C₂₃H₁₆FN₈: 423.1 (M+H)⁺; found: 423.3 (M+H)⁺.

### Example A16: Synthesis of 3-(4-amino-7-(1-methyl-1H-pyrazol-5-yl)-2-(pyridin-2-ylmethyl)-2H-[1,2,3]triazolo[4,5-c]pyridin-6-yl)-2-fluorobenzonitrile (Compound 16)

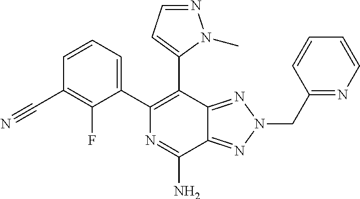 [SMILES: Cn1nccc1-c1c(-c2cccc(C#N)c2F)nc(N)c2nn(Cc3ccccn3)nc12]

### Step 1. 3-(4-amino-7-bromo-2-(pyridin-2-ylmethyl)-2H-[1,2,3]triazolo[4,5-c]pyridin-6-yl)-2-fluorobenzonitrile

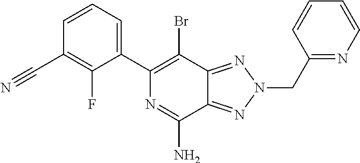 [SMILES: N#Cc1cccc(-c2nc(N)c3nn(Cc4ccccn4)nc3c2Br)c1F]

This compound was prepared by following a similar procedure from Example A13, Step 1 to Step 6, with (3-cyano-2-fluorophenyl)boronic acid replacing (3-cyanophenyl)boronic acid in Step 4. LC-MS calculated for C₁₈H₁₂BrFN₇: 424.0 (M+H)⁺ and 426.0 (M+H)⁺; found: 424.3 (M+H)⁺ and 426.3 (M+H)⁺.

### Step 2. 3-(4-amino-7-(1-methyl-1H-pyrazol-5-yl)-2-(pyridin-2-ylmethyl)-2H-[1,2,3]triazolo[4,5-c]pyridin-6-yl)-2-fluorobenzonitrile

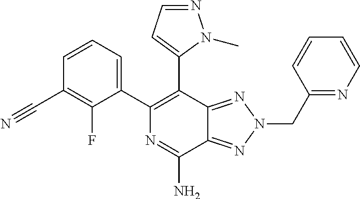 [SMILES: Cn1nccc1-c1c(-c2cccc(C#N)c2F)nc(N)c2nn(Cc3ccccn3)nc12]

This compound was prepared by following a similar procedure in Example A15, with (1-methyl-1H-pyrazol-5-yl)boronic acid replacing pyridin-4-ylboronic acid, and with 3-(4-amino-7-bromo-2-(pyridin-2-ylmethyl)-2H-[1,2,3]triazolo[4,5-c]pyridin-6-yl)-2-fluorobenzonitrile replacing 3-(4-amino-7-bromo-2-((3-fluoropyridin-2-yl)methyl)-2H-[1,2,3]triazolo[4,5-c]pyridin-6-yl)benzonitrile. LC-MS calculated for C₂₂H₁₇FN₉: 426.2 (M+H)⁺; found: 426.3 (M+H)⁺.

### Example A17: Synthesis of 7-(1-((5-Chloropyridin-3-yl)methyl)-1H-pyrazol-4-yl)-3-methyl-9-pentyl-6,9-dihydro-5H-pyrrolo[3,2-d][1,2,4]triazolo[4,3-a]pyrimidin-5-one (Compound 17)

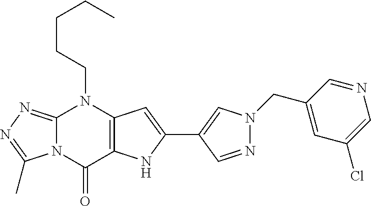 [SMILES: CCCCCn1c2cc(-c3cnn(Cc4cncc(Cl)c4)c3)[nH]c2c(=O)n2c(C)nnc12]

### Step 1: Ethyl 3-(pentylamino)-1H-pyrrole-2-carboxylate

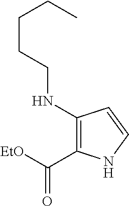 [SMILES: CCCCCNc1cc[nH]c1C(=O)OCC]

Ethyl 3-amino-1H-pyrrole-2-carboxylate (5 g, 32.4 mmol), pentanal (3.79 ml, 35.7 mmol), and sodium cyanoborohydride (2.038 g, 32.4 mmol) were mixed in methanol (64.9 ml) at room temperature overnight. The reaction mixture was concentrated under reduced pressure. The crude residue was purified by flash chromatography (0 to 100% EtOAc in hexanes) to give the desired product (4.4 g, 61%). LCMS calculated for C₁₂H₂₁N₂O₂ (M+H): 225.2. Found: 225.1.

### Step 2: Ethyl 3-(3-(ethoxycarbonyl)-1-pentylthioureido)-1H-pyrrole-2-carboxylate

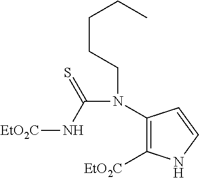 [SMILES: CCCCCN(C(=S)NC(=O)OCC)c1cc[nH]c1C(=O)OCC]

A vial was charged with ethyl 3-(pentylamino)-1H-pyrrole-2-carboxylate (4.4 g, 19.62 mmol), dichloromethane (39.2 ml), and ethoxycarbonyl isothiocyanate (2.78 ml, 23.54 mmol). The reaction mixture was stirred at room temperature overnight. The reaction mixture was quenched with water (40 ml), and the layers were separated. The aqueous layer was extracted with dichloromethane (3×40 mL) and the combined organic fractions were dried over MgSO₄, filtered, and concentrated. The crude material was used in the next step without further purification (7.3 g, quant.). LCMS calculated for C₁₆H₂₆N₃O₄S (M+H): 356.2. Found: 356.1.

### Step 3: 1-Pentyl-2-thioxo-2,3-dihydro-1H-pyrrolo[3,2-d]pyrimidin-4(5H)-one

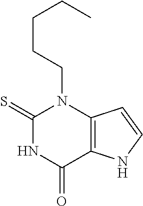 [SMILES: CCCCCn1c(=S)[nH]c(=O)c2[nH]ccc21]

A microwave vial was charged with ethyl 3-(3-(ethoxycarbonyl)-1-pentylthioureido)-1H-pyrrole-2-carboxylate (7.31 g, 20.57 mmol) and sodium ethoxide (21% w/w, 8.45 ml, 22.62 mmol) solution. The vial was capped and heated in a microwave reactor for 10 minutes at 120 degrees Celsius. The reaction mixture was brought to neutral pH on addition of 1M HCl solution and the solid product was filtered and dried (3.1 g, 64%). LCMS calculated for C₁₁H₁₆N₃OS (M+H): 238.1. Found: 238.1.

### Step 4: 2-Hydrazono-1-pentyl-2,3-dihydro-1H-pyrrolo[3,2-d]pyrimidin-4(5H)-one

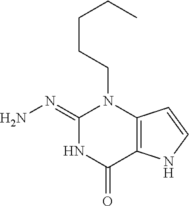 [SMILES: CCCCCn1/c(=N/N)[nH]c(=O)c2[nH]ccc21]

A vial was charged with 1-pentyl-2-thioxo-2,3-dihydro-1H-pyrrolo[3,2-d]pyrimidin-4(5H)-one (3.13 g, 13.19 mmol) and hydrazine hydrate (20 mL). The reaction mixture was stirred at 100 degrees Celsius overnight. The solid formed was filtered and washed with water to give the desired product (2.2 g, 70%). LCMS calculated for C₁₁H₁₈N₅O (M+H): 236.1. Found: 236.1.

### Step 5: 3-Methyl-9-pentyl-6,9-dihydro-5H-pyrrolo[3,2-d][1,2,4]triazolo[4,3-a]pyrimidin-5-one

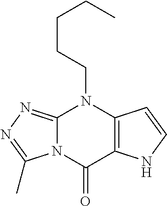 [SMILES: CCCCCn1c2cc[nH]c2c(=O)n2c(C)nnc12]

A vial was charged with (E)-2-hydrazono-1-pentyl-2,3-dihydro-1H-pyrrolo[3,2-d]pyrimidin-4(5H)-one (4.8 g, 20.40 mmol), a drop of trifluoroacetic acid, and triethyl orthoacetate (20 mL). The reaction mixture was heated to 110 degrees Celsius for three hours. The suspension was filtered, washed with hexanes, and dried (4.0 g, 76%). LCMS calculated for C₁₃H₁₈N₅O (M+H): 260.1. Found: 260.2.

### Step 6: 3-Methyl-9-pentyl-6-(phenylsulfonyl)-6,9-dihydro-5H-pyrrolo[3,2-d][1,2,4]triazolo[4,3-a]pyrimidin-5-one

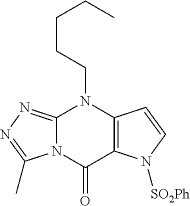

A vial was charged with 3-methyl-9-pentyl-6,9-dihydro-5H-pyrrolo[3,2-d][1,2,4]triazolo[4,3-a]pyrimidin-5-one (from Step 1) (4 g, 15.43 mmol), dichloromethane (40 mL), dimethylaminopyridine (0.188 g, 1.543 mmol), triethylamine (3.23 ml, 23.14 mmol), and benzenesulfonyl chloride (2.187 ml, 16.97 mmol). The reaction mixture was stirred at room temperature for one hour. The reaction mixture was quenched with water, and the layers were separated. The aqueous layer was extracted with dichloromethane (3×40 mL) and the combined organic fractions were dried over MgSO₄, filtered, and concentrated. The crude material was used in the next step without further purification (6.1 g, quant.). LCMS calculated for C₁₉H₂₂N₅O₃S (M+H): 400.1. Found: 400.1.

### Step 7: 7-Bromo-3-methyl-9-pentyl-6-(phenylsulfonyl)-6,9-dihydro-5H-pyrrolo[3,2-d][1,2,4]triazolo[4,3-a]pyrimidin-5-one

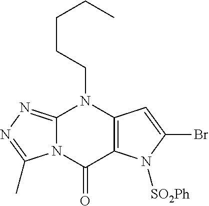

A vial was charged with 3-methyl-9-pentyl-6-(phenylsulfonyl)-6,9-dihydro-5H-pyrrolo[3,2-d][1,2,4]triazolo[4,3-a]pyrimidin-5-one (1 g, 2.503 mmol), dry THF (30 mL) and the mixture was cooled to −78 degrees Celsius. Lithium diisopropylamide solution (1M in hexanes/THF, 3.13 ml, 3.13 mmol) was added dropwise. The reaction mixture was maintained at −78° C. for 1.5 hours. A solution of 1,2-dibromo-1,1,2,2-tetrachloroethane (1.223 g, 3.75 mmol) in dry THF (3 ml) was added dropwise to the reaction mixture and the reaction mixture was maintained at −78° C. for a further 1.5 hours. The reaction mixture was quenched with sat. aq. NH₄Cl solution (30 mL) and diluted with dichloromethane (30 mL). The layers were separated and the aqueous layer was extracted with DCM (3×30 mL). The combined organic fractions were dried over MgSO₄, filtered, and concentrated. The crude residue was purified by automated flash chromatography (0 to 100% EtOAc in DCM) to give the desired product (0.84 g, 70%). LCMS calculated for C₁₉H₂₁BrN₅O₃S (M+H): 478.1. Found: 478.1.

### Step 8: 3-Chloro-5-((4-(4,4,5,5-tetramethyl-1,3,2-dioxaborolan-2-yl)-1H-pyrazol-1-yl)methyl)pyridine

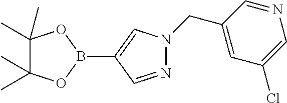 [SMILES: CC1(C)OB(c2cnn(Cc3cncc(Cl)c3)c2)OC1(C)C]

A vial was charged with 4-(4,4,5,5-tetramethyl-1,3,2-dioxaborolan-2-yl)-1H-pyrazole (0.5 g, 2.58 mmol), 3-(bromomethyl)-5-chloropyridine hydrobromide (0.741 g, 2.58 mmol), cesium carbonate (2.52 g, 7.73 mmol), and DMF (6.44 ml). The reaction mixture was stirred at 60 degrees Celsius for one hour. The reaction mixture was quenched with water (10 ml) and diluted with dichloromethane (10 ml). The layers were separated, and the aqueous layer was extracted with dichloromethane (3×10 mL). The combined dichloromethane extracts were dried over MgSO₄, filtered, and concentrated. Purification by automated flash chromatography (0 to 100% EtOAc in DCM) afforded the product (0.548 g, 67%). LCMS calculated for C₁₅H₂₀BClN₃O₂(M+H): 320.1, 322.1. Found: 320.1, 322.1.

### Step 9: 7-(1-((5-Chloropyridin-3-yl)methyl)-1H-pyrazol-4-yl)-3-methyl-9-pentyl-6,9-dihydro-5H-pyrrolo[3,2-d][1,2,4]triazolo[4,3-a]pyrimidin-5-one

A vial was charged with 7-bromo-3-methyl-9-pentyl-6-(phenylsulfonyl)-6,9-dihydro-5H-pyrrolo[3,2-d][1,2,4]triazolo[4,3-a]pyrimidin-5-one (0.01 g, 0.021 mmol), 3-chloro-5-((4-(4,4,5,5-tetramethyl-1,3,2-dioxaborolan-2-yl)-1H-pyrazol-1-yl)methyl)pyridine (0.013 g, 0.042 mmol), Chloro(2-dicyclohexylphosphino-2′,4′,6′-triisopropyl-1,1′-biphenyl)[2-(2′-amino-1,1′-biphenyl)]palladium(II) (5.00 mg, 0.006 mmol) and potassium phosphate tribasic (0.016 g, 0.074 mmol). 1,4-dioxane (0.35 ml) and water (0.07 ml) were added and the reaction mixture was sparged with nitrogen gas for 5 minutes then stirred at 90° C. for two hours. The reaction mixture was cooled to room temperature and sodium hydroxide (10 mg) was added. The reaction mixture was stirred at 40 degrees Celsius for 60 minutes. The reaction mixture was cooled to room temperature and diluted with DMF (5 ml). Purification by preparative HPLC (pH 2, acetonitrile/water with TFA) afforded the product as a TFA salt (2 mg, 21%). LCMS calculated for C₂₂H₂₄ClN₈O (M+H): 451.2, 453.2. Found: 451.2, 453.2.

### Example A18: Synthesis of 3-Methyl-7-(1-((5-methylpyridin-3-yl)methyl)-1H-pyrazol-4-yl)-9-pentyl-6,9-dihydro-5H-pyrrolo[3,2-d][1,2,4]triazolo[4,3-a]pyrimidin-5-one (Compound 18)

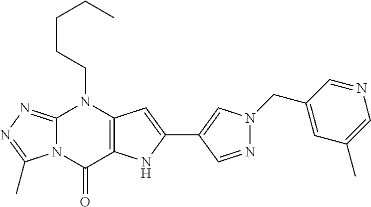 [SMILES: CCCCCn1c2cc(-c3cnn(Cc4cncc(C)c4)c3)[nH]c2c(=O)n2c(C)nnc12]

This compound was prepared using similar procedures as described in Example A17 using 3-(bromomethyl)-5-methylpyridine in place of 3-(bromomethyl)-5-chloropyridine hydrobromide in Step 8. LCMS calculated for C₂₃H₂₇N₈O (M+H): 431.2. Found: 431.3.

### Example A19: Synthesis of 3-Methyl-9-pentyl-7-(1-(thieno[3,2-b]pyridin-6-ylmethyl)-1H-pyrazol-4-yl)-6,9-dihydro-5H-pyrrolo[3,2-d][1,2,4]triazolo[4,3-a]pyrimidin-5-one (Compound 19)

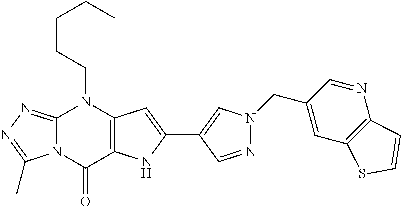 [SMILES: CCCCCn1c2cc(-c3cnn(Cc4cnc5ccsc5c4)c3)[nH]c2c(=O)n2c(C)nnc12]

This compound was prepared using similar procedures as described in Example A17 using 6-(bromomethyl)thieno[3,2-b]pyridine in place of 3-(bromomethyl)-5-chloropyridine hydrobromide in Step 8. LCMS calculated for C₂₄H₂₅N₈OS (M+H): 473.2. Found: 473.3.

### Example A20: 7-(1-((2-(2-(Dimethylamino)acetyl)-1,2,3,4-tetrahydroisoquinolin-6-yl)methyl)-1H-pyrazol-4-yl)-3-methyl-9-pentyl-6,9-dihydro-5H-pyrrolo[3,2-d][1,2,4]triazolo[4,3-a]pyrimidin-5-one (Compound 20)

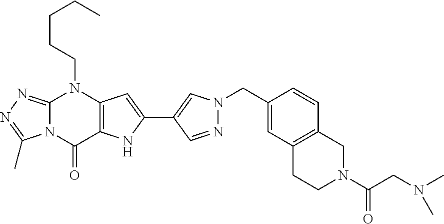 [SMILES: CCCCCn1c2cc(-c3cnn(Cc4ccc5c(c4)CCN(C(=O)CN(C)C)C5)c3)[nH]c2c(=O)n2c(C)nnc12]

### Step 1: tert-Butyl 6-((4-(4,4,5,5-tetramethyl-1,3,2-dioxaborolan-2-yl)-1H-pyrazol-1-yl)methyl)-3,4-dihydroisoquinoline-2(1H)-carboxylate

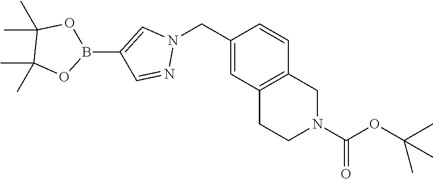 [SMILES: CC(C)(C)OC(=O)N1CCc2cc(Cn3cc(B4OC(C)(C)C(C)(C)O4)cn3)ccc2C1]

A flask was charged with 4-(4,4,5,5-tetramethyl-1,3,2-dioxaborolan-2-yl)-1H-pyrazole (0.5 g, 2.58 mmol), tert-butyl 6-(hydroxymethyl)-3,4-dihydroisoquinoline-2(1H)-carboxylate (0.339 g, 1.288 mmol), triphenylphosphine (0.743 g, 2.83 mmol), and THF (12 ml). The solution was cooled to 0° C. and DIAD (0.601 ml, 3.09 mmol) was added dropwise. The reaction mixture was stirred overnight at room temperature. The mixture was diluted with ethyl acetate and washed with water, dried and concentrated. The product was purified by column chromatography eluting with Hexane/EtOAc (max. EtOAc 60%) to afford the product. LCMS calculated for C₂₄H₃₅BN₃O₄(M+H)⁺: m/z=440.3; found 440.3.

### Step 2: 7-bromo-3-methyl-9-pentyl-6,9-dihydro-5H-pyrrolo[3,2-d][1,2,4]triazolo[4,3-a]pyrimidin-5-one

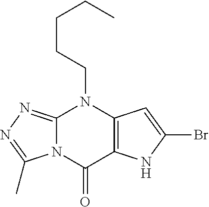 [SMILES: CCCCCn1c2cc(Br)[nH]c2c(=O)n2c(C)nnc12]

TBAF (1.0 M in THF) (2.0 ml, 2.0 mmol) was added to a solution of 7-bromo-3-methyl-9-pentyl-6-(phenylsulfonyl)-6,9-dihydro-5H-pyrrolo[3,2-d][1,2,4]triazolo[4,3-a]pyrimidin-5-one (0.360 g, 0.753 mmol) in THF (4.0 ml), and then the reaction was stirred at 50° C. for 1 h. The solvent was removed and the product was purified by column 15 chromatography eluting with CH₂Cl₂/MeOH (max. MeOH 10%). LCMS calculated for C₁₃H₁₇BrN₅O (M+H)⁺: m/z=338.1; found 338.1.

### Step 3: tert-Butyl 6-((4-(3-methyl-5-oxo-9-pentyl-6,9-dihydro-5H-pyrrolo[3,2-d][1,2,4]triazolo[4,3-a]pyrimidin-7-yl)-1H-pyrazol-1-yl)methyl)-3,4-dihydroisoquinoline-2(1H)-carboxylate

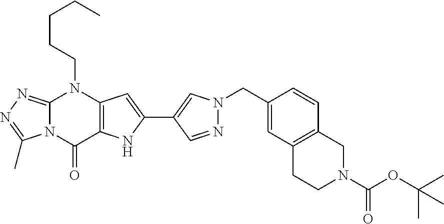 [SMILES: CCCCCn1c2cc(-c3cnn(Cc4ccc5c(c4)CCN(C(=O)OC(C)(C)C)C5)c3)[nH]c2c(=O)n2c(C)nnc12]

A mixture of 7-bromo-3-methyl-9-pentyl-6,9-dihydro-5H-pyrrolo[3,2-d][1,2,4]triazolo[4,3-a]pyrimidin-5-one (from Example A20, Step 2) (0.040 g, 0.118 mmol), tert-butyl 6-((4-(4,4,5,5-tetramethyl-1,3,2-dioxaborolan-2-yl)-1H-pyrazol-1-yl)methyl)-3,4-dihydroisoquinoline-2(1H)-carboxylate (0.062 g, 0.142 mmol), dichloro[1,1′-bis(dicyclohexylphosphino)ferrocene]palladium(II), dichloromethane adduct (Pd-127) (8.94 mg, 0.012 mmol) and cesium fluoride (0.090 g, 0.591 mmol) in t-BuOH (1.5 ml)/Water (0.6 ml) was vacuumed and replaced with N₂ for 3 times. The reaction was then stirred at 105° C. for 2 h, cooled to rt, diluted with ethyl acetate, washed with water, dried and concentrated. The product was purified by column eluting with CH₂Cl₂/MeOH (max. MeOH 10%). LCMS calculated for C₃₁H₃₉N₈O₃ (M+H)⁺: m/z=571.3; found 571.5.

### Step 4: 3-Methyl-9-pentyl-7-(1-((1,2,3,4-tetrahydroisoquinolin-6-yl)methyl)-1H-pyrazol-4-yl)-6,9-dihydro-5H-pyrrolo[3,2-d][1,2,4]triazolo[4,3-a]pyrimidin-5-one

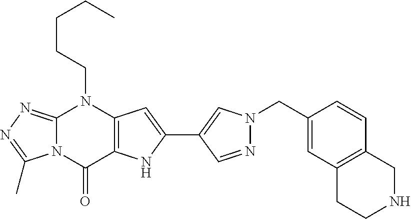 [SMILES: CCCCCn1c2cc(-c3cnn(Cc4ccc5c(c4)CCNC5)c3)[nH]c2c(=O)n2c(C)nnc12]

TFA (0.5 ml, 6.49 mmol) was added to a solution of tert-butyl 6-((4-(3-methyl-5-oxo-9-pentyl-6,9-dihydro-5H-pyrrolo[3,2-d][1,2,4]triazolo[4,3-a]pyrimidin-7-yl)-1H-pyrazol-1-yl)methyl)-3,4-dihydroisoquinoline-2(1H)-carboxylate (50.0 mg, 0.088 mmol) in CH₂Cl₂ (0.5 ml), and then the reaction was stirred at room temperature for 30 min. The solvent was then removed to provide the crude product as TFA salt. LCMS calculated for C₂₆H₃₁N₈O (M+H)⁺: m/z=471.3; found 471.2.

### Step 5: 7-(1-((2-(2-(Dimethylamino)acetyl)-1,2,3,4-tetrahydroisoquinolin-6-yl)methyl)-1H-pyrazol-4-yl)-3-methyl-9-pentyl-6,9-dihydro-5H-pyrrolo[3,2-d][1,2,4]triazolo[4,3-a]pyrimidin-5-one

Dimethylglycinoyl chloride (3.10 mg, 0.026 mmol) was added to a solution of 3-methyl-9-pentyl-7-(1-((1,2,3,4-tetrahydroisoquinolin-6-yl)methyl)-1H-pyrazol-4-yl)-6,9-dihydro-5H-pyrrolo[3,2-d][1,2,4]triazolo[4,3-a]pyrimidin-5-one (6.0 mg, 0.013 mmol) and triethylamine (8.89 μl, 0.064 mmol) in CH₂Cl₂ (0.8 ml) at room temperature and stirred for 30 min. The solvent was removed, and the mixture was diluted with acetonitrile/water and purified by prep HPLC (pH 2, acetonitrile/water with TFA) to provide the desired compound as its TFA salt. LC-MS calculated for C₃₀H₃₈N₉O₂ (M+H)⁺: m/z=556.3; found 556.3.

### Example A21. 3-(2-((5-(1H-pyrazol-1-yl)-2H-tetrazol-2-yl)methyl)-5-amino-8-(pyrimidin-4-yl)-[1,2,4]triazolo[1,5-c]pyrimidin-7-yl)benzonitrile (Compound 21A) and 3-(2-((5-(1H-Pyrazol-1-yl)-1H-tetrazol-1-yl)methyl)-5-amino-8-(pyrimidin-4-yl)-[1,2,4]triazolo[1,5-c]pyrimidin-7-yl)benzonitrile (Compound 21B)

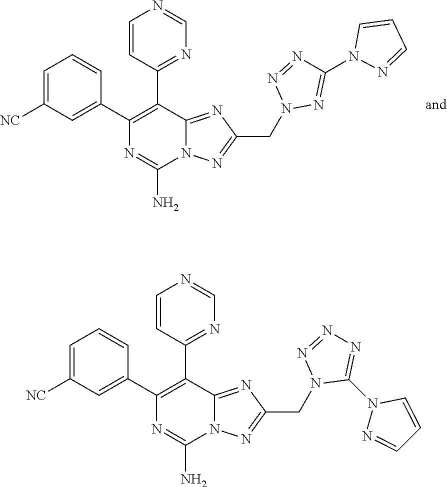 [SMILES: N#Cc1cccc(-c2nc(N)n3nc(Cn4nnc(-n5cccn5)n4)nc3c2-c2ccncn2)c1 ; N#Cc1cccc(-c2nc(N)n3nc(Cn4nnnc4-n4cccn4)nc3c2-c2ccncn2)c1]

The mixture of title compounds was prepared using similar procedures as described for Example A3, with 5-(1H-pyrazol-1-yl)-1H-tetrazole replacing 2-(1H-tetrazol-5-yl)pyridine. Compound 21A was purified by preparative LC-MS (pH 2, acetonitrile/water with TFA) to afford the product as a TFA salt. LCMS calculated for C₂₁H₁₅N₁₄ (M+H)⁺: 463.2; found 463.2.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A method of treating a cancer in a subject, comprising administering to the subject:
(i) an inhibitor of A2A/A2B; and
(ii) an inhibitor of PD-1/PD-L1.

2. The method of claim 1, wherein the inhibitor of A2A/A2B is a compound of Formula (I): 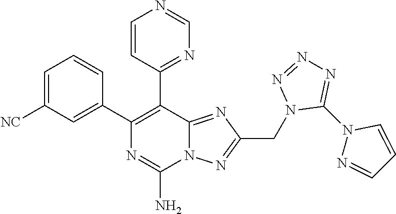 [SMILES: N#Cc1cccc(-c2nc(N)n3nc(Cn4nnnc4-n4cccn4)nc3c2-c2ccncn2)c1]
or a pharmaceutically acceptable salt thereof, wherein
Cy¹ is phenyl which is substituted by 1 or 2 substituents independently selected from halo and CN;
Cy² is 5-6 membered heteroaryl or 4-7 membered heterocycloalkyl, wherein the 5-6 membered heteroaryl or 4-7 membered heterocycloalkyl of Cy² are each optionally substituted with 1, 2, or 3 groups each independently selected from C₁₋₃ alkyl, C₁₋₃ alkoxy, NH₂, NH(C₁₋₃ alkyl) and N(C₁₋₃ alkyl)₂;
R² is selected from phenyl-C₁₋₃ alkyl-, C₃₋₇ cycloalkyl-C₁₋₃ alkyl-, (5-7 membered heteroaryl)-C₁₋₃ alkyl-, (4-7 membered heterocycloalkyl)-C₁₋₃ alkyl-, and OR^{a2}, wherein the phenyl-C₁₋₃ alkyl-, C₃₋₇ cycloalkyl-C₁₋₃ alkyl-, (5-7 membered heteroaryl)-C₁₋₃ alkyl-, and (4-7 membered heterocycloalkyl)-C₁₋₃ alkyl-of R² are each optionally substituted with 1, 2, or 3 independently selected R^{C} substituents;
R^{a2} is (5-7 membered heteroaryl)-C₁₋₃ alkyl-optionally substituted with 1 or 2 independently selected R^{C} substituents;
each R^{C} is independently selected from halo, C₁₋₆ alkyl, C₆ aryl, 5-7 membered heteroaryl, (4-7 membered heterocycloalkyl)-C₁₋₃ alkyl-, OR^{a4}, and NR^{c4}R^{d4}; and
each R^{a4}, R^{c4}, and R^{d4} are independently selected from H and C₁₋₆ alkyl.

3. The method of claim 1, wherein the inhibitor of A2A/A2B is selected from:
3-(5-Amino-2-(pyridin-2-ylmethyl)-8-(pyrimidin-4-yl)-[1,2,4]triazolo[1,5-c]pyrimidin-7-yl)benzonitrile, or a pharmaceutically acceptable salt thereof,
3-(5-Amino-2-((2,6-difluorophenyl)(hydroxy)methyl)-8-(pyrimidin-4-yl)-[1,2,4]triazolo[1,5-c]pyrimidin-7-yl)benzonitrile, or a pharmaceutically acceptable salt thereof,
3-(5-Amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof,
3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile, or a pharmaceutically acceptable salt thereof,
3-(5-Amino-2-((3-methylpyridin-2-yl)methoxy)-8-(pyrimidin-4-yl)-[1,2,4]triazolo[1,5-c]pyrimidin-7-yl)benzonitrile, or a pharmaceutically acceptable salt thereof; and
3-(2-((5-(1H-Pyrazol-1-yl)-2H-tetrazol-2-yl)methyl)-5-amino-8-(pyrimidin-4-yl)-[1,2,4]triazolo[1,5-c]pyrimidin-7-yl)benzonitrile, or a pharmaceutically acceptable salt thereof.

4. The method of claim 1, wherein the inhibitor of A2A/A2B is a compound of Formula (II): 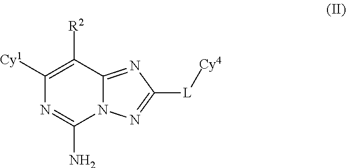
or a pharmaceutically acceptable salt thereof, wherein
R² is selected from H and CN;
Cy¹ is phenyl which is substituted by 1 or 2 substituents independently selected from halo and CN;
L is C₁₋₃ alkylene, wherein said alkylene is optionally substituted with 1, 2, or 3 independently selected R^{8D} substituents;
Cy⁴ is selected from phenyl, cyclohexyl, pyridyl, pyrrolidinonyl, and imidazolyl, wherein the phenyl, cyclohexyl, pyridyl, pyrrolidinonyl, and imidazolyl are each optionally substituted with 1, 2, or 3 substituents independently selected from R^{8D} and R⁸;
each R⁸ is independently selected from halo, C₁₋₆ alkyl, C₁₋₆ haloalkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl, wherein the C₁₋₆ alkyl, C₂₋₄ alkenyl, C₂₋₄ alkynyl, phenyl, C₃₋₇ cycloalkyl, 5-6 membered heteroaryl, 4-7 membered heterocycloalkyl, phenyl-C₁₋₃ alkyl, C₃₋₇ cycloalkyl-C₁₋₃ alkyl, (5-6 membered heteroaryl)-C₁₋₃ alkyl, and (4-7 membered heterocycloalkyl)-C₁₋₃ alkyl of R⁸ are each optionally substituted with 1, 2, or 3 independently selected R^{8A} substituents;
each R^{8A} is independently selected from halo, C₁₋₆ alkyl, 5-6 membered heteroaryl, 4-7 membered heterocycloalkyl, CN, OR^{a81}, and NR^{c81}R^{d81}, wherein the C₁₋₃ alkyl, 5-6 membered heteroaryl, and 4-7 membered heterocycloalkyl of R^{8A} are each optionally substituted with 1, 2, or 3 independently selected R^{8B} substituents;
each R^{a81}, R^{c81}, and R^{d81} is independently selected from H, C₁₋₆ alkyl, and 4-7 membered heterocycloalkyl, wherein the C₁₋₆ alkyl and 4-7 membered heterocycloalkyl of R^{a81}, R^{c81}, and R^{d81} are each optionally substituted with 1, 2, or 3 independently selected R^{8B} substituents;
each R^{8B} is independently selected from halo and C₁₋₃ alkyl; and
each R^{8D} is independently selected from OH, CN, halo, C₁₋₆ alkyl, and C₁₋₆ haloalkyl.

5. The method of claim 1, wherein the inhibitor of A2A/A2B is selected from:
3-(5-Amino-2-(hydroxy(phenyl)methyl)-[1,2,4]triazolo[1,5-c]pyrimidin-7-yl)benzonitrile, or a pharmaceutically acceptable salt thereof,
3-(5-Amino-2-((2,6-difluorophenyl)(hydroxy)methyl)-[1,2,4]triazolo[1,5-c]pyrimidin-7-yl)-2-fluorobenzonitrile, or a pharmaceutically acceptable salt thereof:
5-Amino-7-(3-cyano-2-fluorophenyl)-2-((2,6-difluorophenyl)(hydroxy)methyl)-[1,2,4]triazolo[1,5-c]pyrimidine-8-carbonitrile, or a pharmaceutically acceptable salt thereof; and
3-(5-Amino-2-((2-fluoro-6-(((1-methyl-2-oxopyrrolidin-3-yl)amino)methyl)phenyl)(hydroxy)methyl)-[1,2,4]triazolo[1,5-c]pyrimidin-7-yl)-2-fluorobenzonitrile, or a pharmaceutically acceptable salt thereof.

6. The method of claim 1, wherein the inhibitor of A2A/A2B is a compound of Formula (III): 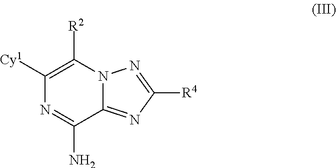
or a pharmaceutically acceptable salt thereof, wherein
Cy¹ is phenyl which is substituted by 1 or 2 substituents independently selected from halo and CN;
R² is selected from 5-6 membered heteroaryl and 4-7 membered heterocycloalkyl, wherein the 5-6 membered heteroaryl and 4-7 membered heterocycloalkyl of R² are each optionally substituted with 1, 2, or 3 independently selected R^{2A} substituents;
each R^{2A} is independently selected from D, halo, C₁₋₆ alkyl, and C₁₋₆ haloalkyl;
R⁴ is selected from phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl wherein the phenyl-C₁₋₃ alkyl-, C₃₋₇ cycloalkyl-C₁₋₃ alkyl-, (5-6 membered heteroaryl)-C₁₋₃ alkyl-, and (4-7 membered heterocycloalkyl)-C₁₋₃ alkyl-of R⁴ are each optionally substituted with 1, 2, or 3 independently selected R^{4A} substituents;
each R^{4A} is independently selected from halo, C₁₋₆ alkyl, C₁₋₆ haloalkyl, CN, OR^{a41}, and NR^{c41}R^{d41}; and
each R^{a41}, R^{c41} and R^{d41} is independently selected from H and C₁₋₆ alkyl.

7. The method of claim 1, wherein the inhibitor of A2A/A2B is selected from:
3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile;
3-(8-Amino-2-((2,6-difluorophenyl)(hydroxy)methyl)-5-(pyrimidin-4-yl)-[1,2,4]triazolo[1,5-a]pyrazin-6-yl)benzonitrile, or a pharmaceutically acceptable salt thereof,
3-(8-amino-2-(amino(2,6-difluorophenyl)methyl)-5-(4-methyloxazol-5-yl)-[1,2,4]triazolo[1,5-a]pyrazin-6-yl)benzonitrile, or a pharmaceutically acceptable salt thereof, and
3-(8-amino-2-((2,6-difluorophenyl)(hydroxy)methyl)-5-(2,6-dimethylpyridin-4-yl)-[1,2,4]triazolo[1,5-a]pyrazin-6-yl)benzonitrile, or a pharmaceutically acceptable salt thereof.

8. The method of claim 1, wherein the inhibitor of A2A/A2B is a compound of Formula (IV): 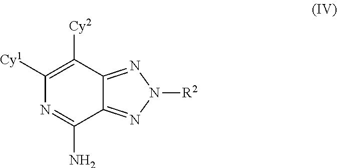
or a pharmaceutically acceptable salt thereof, wherein
Cy¹ is phenyl which is substituted by 1 or 2 substituents independently selected from halo and CN;
Cy² is selected from 5-6 membered heteroaryl and 4-7 membered heterocycloalkyl, wherein the 5-6 membered heteroaryl and 4-7 membered heterocycloalkyl of Cy² are each optionally substituted with 1, 2, or 3 independently selected R⁶ substituents;
each R⁶ is independently selected from halo, C₁₋₆ alkyl, and C₁₋₆ haloalkyl;
R² is phenyl-C₁₋₃ alkyl- or (5-6 membered heteroaryl)-C₁₋₃ alkyl-, wherein the phenyl-C₁₋₃ alkyl- and (5-6 membered heteroaryl)-C₁₋₃ alkyl-of R² are each optionally substituted with 1, 2, or 3 independently selected R^{2A} substituents; and
each R^{2A} is independently selected from halo, C₁₋₆ alkyl, and C₁₋₆ haloalkyl.

9. The method of claim 1, wherein the inhibitor of A2A/A2B is selected from:
3-(4-amino-2-(pyridin-2-ylmethyl)-7-(pyrimidin-4-yl)-2H-[1,2,3]triazolo[4,5-c]pyridin-6-yl)benzonitrile, or a pharmaceutically acceptable salt thereof;
3-(4-amino-2-((3-fluoropyridin-2-yl)methyl)-7-(pyrimidin-4-yl)-2H-[1,2,3]triazolo[4,5-c]pyridin-6-yl)benzonitrile, or a pharmaceutically acceptable salt thereof;
3-(4-amino-2-((3-fluoropyridin-2-yl)methyl)-7-(pyridin-4-yl)-2H-[1,2,3]triazolo[4,5-c]pyridin-6-yl)benzonitrile, or a pharmaceutically acceptable salt thereof; and
3-(4-amino-7-(1-methyl-1H-pyrazol-5-yl)-2-(pyridin-2-ylmethyl)-2H-[1,2,3]triazolo[4,5-c]pyridin-6-yl)-2-fluorobenzonitrile, or a pharmaceutically acceptable salt thereof.

10. The method of claim 1, wherein the inhibitor of A2A/A2B is 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof.

11. The method of claim 1, wherein the inhibitor of A2A/A2B is 3-(5-Amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof.

12. The method of claim 1, wherein the inhibitor of PD-1/PD-L1 is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof.

13. The method of claim 1, wherein the inhibitor of PD-1/PD-L1 is pembrolizumab.

14. The method of claim 1, wherein the inhibitor of PD-1/PD-L1 is atezolizumab.

15. The method of claim 1, wherein the inhibitor of PD-1/PD-L1 is ANTIBODY X, wherein ANTIBODY X is an antibody or antigen-binding fragment thereof comprises a variable heavy (VH) domain comprising VH complementarity determining region (CDR)1, VH CDR2, and VH CDR3, wherein:
the VH CDR1 comprises the amino acid sequence SYWMN (SEQ ID NO:6);
the VH CDR2 comprises the amino acid sequence VIIIPSDSETWLDQKFKD (SEQ ID NO:7); and
the VH CDR3 comprises the amino acid sequence EHYGTSPFAY (SEQ ID NO:8); and
wherein the antibody comprises a variable light (VL) domain comprising VL CDR1, VL CDR2, and VL CDR3, wherein:
the VL CDR1 comprises the amino acid sequence RASESVDNYGMSFMNW (SEQ ID NO:9);
the VL CDR2 comprises the amino acid sequence AASNQGS (SEQ ID NO:10); and
the VL CDR3 comprises the amino acid sequence QQSKEVPYT (SEQ ID NO:11).

16. The method of claim 15, wherein ANTIBODY X is a humanized antibody.

17. The method of claim 1, wherein the inhibitor of A2A/A2B is administered to the subject in a dosage of from about 0.1 mg to about 1000 mg on a free base basis.

18. The method of claim 1, wherein the A2A/A2B inhibitor is administered to the subject once-daily, every other day, or once-weekly.

19. The method of claim 1, wherein the inhibitor of A2A/A2B and inhibitor of PD-1/PD-L1 are administered simultaneously.

20. The method of claim 1, wherein the inhibitor of A2A/A2B and inhibitor of PD-1/PD-L1 are administered sequentially.

21. The method of claim 1, wherein the cancer is selected from bladder cancer, breast cancer, head and neck cancer, lung cancer, ovarian cancer, pancreatic cancer, prostate cancer, and colorectal cancer.

22.-24. (canceled)

25. A method of treating a cancer selected from bladder cancer, breast cancer, head and neck cancer, lung cancer, ovarian cancer, pancreatic cancer, prostate cancer, and colorectal cancer in a subject, comprising administering to the subject:
(i) an inhibitor of A2A/A2B which is 3-(8-Amino-5-(1-methyl-6-oxo-1,6-dihydropyridazin-3-yl)-2-(pyridin-2-ylmethyl)-[1,2,4]triazolo[1,5-a]pyrazin-6-yl)benzonitrile, or a pharmaceutically acceptable salt thereof; and
(ii) an inhibitor of PD-1/PD-L1 which is ANTIBODY X;
wherein the inhibitor of A2A/A2B is administered to the subject in a dosage of from about 0.1 mg to about 500 mg on a free base basis, wherein the inhibitor of A2A/A2B is administered once-daily or every other day; and
the ANTIBODY X is administered to the subject in a dosage of about 100 mg to about 1000 mg Q4W;
wherein ANTIBODY X is an antibody or antigen-binding fragment thereof comprises a variable heavy (VH) domain comprising VH complementarity determining region (CDR)1, VH CDR2, and VH CDR3, wherein:
the VH CDR1 comprises the amino acid sequence SYWMN (SEQ ID NO:6);
the VH CDR2 comprises the amino acid sequence VIIIPSDSETWLDQKFKD (SEQ ID NO:7); and
the VH CDR3 comprises the amino acid sequence EHYGTSPFAY (SEQ ID NO:8); and
wherein the antibody comprises a variable light (VL) domain comprising VL CDR1, VL CDR2, and VL CDR3, wherein:
the VL CDR1 comprises the amino acid sequence RASESVDNYGMSFMNW (SEQ ID NO:9);
the VL CDR2 comprises the amino acid sequence AASNQGS (SEQ ID NO:10); and
the VL CDR3 comprises the amino acid sequence QQSKEVPYT (SEQ ID NO:11).

26. A method of treating a cancer selected from bladder cancer, breast cancer, head and neck cancer, lung cancer, ovarian cancer, pancreatic cancer, prostate cancer, and colorectal cancer in a subject, comprising administering to the subject:
(i) an inhibitor of A2A/A2B which is 3-(5-Amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile, or a pharmaceutically acceptable salt thereof; and
(ii) an inhibitor of PD-1/PD-L1 which is ANTIBODY X;
wherein the inhibitor of A2A/A2B is administered to the subject in a dosage of from about 0.1 mg to about 500 mg on a free base basis, wherein the inhibitor of A2A/A2B is administered once-daily or every other day; and
the ANTIBODY X is administered to the subject in a dosage of about 100 mg to about 1000 mg Q4W;
wherein ANTIBODY X is an antibody or antigen-binding fragment thereof comprises a variable heavy (VH) domain comprising VH complementarity determining region (CDR)1, VH CDR2, and VH CDR3, wherein:
the VH CDR1 comprises the amino acid sequence SYWMN (SEQ ID NO:6);
the VH CDR2 comprises the amino acid sequence VIHPSDSETWLDQKFKD (SEQ ID NO:7); and
the VH CDR3 comprises the amino acid sequence EHYGTSPFAY (SEQ ID NO:8); and
wherein the antibody comprises a variable light (VL) domain comprising VL CDR1, VL CDR2, and VL CDR3, wherein:
the VL CDR1 comprises the amino acid sequence RASESVDNYGMSFMNW (SEQ ID NO:9);
the VL CDR2 comprises the amino acid sequence AASNQGS (SEQ ID NO:10); and
the VL CDR3 comprises the amino acid sequence QQSKEVPYT (SEQ ID NO:11).

27. The method of claim 21, wherein the cancer is head and neck cancer.

28. The method of claim 27, wherein the head and neck cancer is head and neck squamous cell carcinoma.

29. The method of claim 21, wherein the cancer is lung cancer.

30. The method of claim 29, wherein the lung cancer is non-small cell lung cancer (NSCLC).

31. The method of claim 21, wherein the cancer is ovarian cancer.

32. The method of claim 21, wherein the cancer is prostate cancer.

33. The method of claim 32, wherein the prostate cancer is metastatic castration-resistant prostate cancer.

34. The method of claim 21, wherein the cancer is breast cancer.

35. The method of claim 21, wherein the cancer is bladder cancer.

36. The method of claim 21, wherein the cancer is colorectal cancer.

37. The method of claim 21, wherein the cancer is pancreatic cancer.


SEQUENCE LISTING (11 sequences)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 445 aa, synthetic construct
  REGION 1..445: note = Description of Artificial Sequence: Syntheticpolypeptide
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 3 - protein, 218 aa, synthetic construct
  REGION 1..218: note = Description of Artificial Sequence: Syntheticpolypeptide
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 119 aa, synthetic construct
  REGION 1..119: note = Description of Artificial Sequence: Syntheticpolypeptide
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 5 - protein, 111 aa, synthetic construct
  REGION 1..111: note = Description of Artificial Sequence: Syntheticpolypeptide
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 6 - protein, 5 aa, synthetic construct
  REGION 1..5: note = Description of Artificial Sequence: Syntheticpeptide
SYWMN

SEQ ID NO: 7 - protein, 17 aa, synthetic construct
  REGION 1..17: note = Description of Artificial Sequence: Syntheticpeptide
VIHPSDSETWLDQKFKD

SEQ ID NO: 8 - protein, 10 aa, synthetic construct
  REGION 1..10: note = Description of Artificial Sequence: Syntheticpeptide
EHYGTSPFAY

SEQ ID NO: 9 - protein, 16 aa, synthetic construct
  REGION 1..16: note = Description of Artificial Sequence: Syntheticpeptide
RASESVDNYGMSFMNW

SEQ ID NO: 10 - protein, 7 aa, synthetic construct
  REGION 1..7: note = Description of Artificial Sequence: Syntheticpeptide
AASNQGS

SEQ ID NO: 11 - protein, 9 aa, synthetic construct
  REGION 1..9: note = Description of Artificial Sequence: Syntheticpeptide
QQSKEVPYT